(19) United States
(12) Patent Application Publication
(10) Pub. No.: US 2022/0112504 A1
(43) Pub. Date: Apr. 14, 2022

(54) METHODS AND COMPOSITIONS FOR ALLELE SPECIFIC GENE EDITING

(71) Applicants: Children's Medical Center Corporation, Boston, MA (US); President and Fellows of Harvard College, Cambridge, MA (US)

(72) Inventors: Jeffrey R. HOLT, Boston, MA (US); Bence GYORGY, Cambridge, MA (US)

(73) Assignees: Children's Medical Center Corporation, Boston, MA (US); President and Fellows of Harvard College, Cambridge, MA (US)

(21) Appl. No.: 17/556,463

(22) Filed: Dec. 20, 2021

Related U.S. Application Data
(63) Continuation of application No. PCT/US2020/038516, filed on Jun. 18, 2020.
(60) Provisional application No. 62/864,933, filed on Jun. 21, 2019.

(51) Int. Cl. C12N 15/113; A61P 27/16; C12N 9/22; C12N 15/86

(52) U.S. Cl. CPC C12N 15/1138; A61P 27/16; C12N 9/22; C12N 2310/20; C12N 2750/14143; C12N 2330/51; C12N 2320/34; C12N 15/86

(57) ABSTRACT

The invention provides compositions and methods for allele specific gene editing. In particular, the invention provides methods and compositions for treating dominant progressive hearing loss by selectively inactivating a dominant mutation in TMC1.

### CROSS-REFERENCE TO RELATED APPLICATIONS

This application is a U.S. National Stage application and a continuation pursuant to 35 U.S.C. §111 of PCT International Patent Application No.: PCT/US2020/038516, filed Jun. 18, 2020, designating the United States and published in English, which claims priority to and the benefit of U.S. Provisional Application No. 62/864,933, filed Jun. 21, 2019, the contents of each of which areincorporated herein by reference in their entirety.

### STATEMENT OF RIGHTS TO INVENTIONS MADE UNDER FEDERALLY SPONSORED RESEARCH

This invention was made with government support under Grant Nos. DC013521 and DC005439 awarded by the National Institutes of Health. The government has certain rights in the invention.

### REFERENCE TO SEQUENCE LISTING

The instant application contains a Sequence Listing which has been submitted electronically in ASCII format and is hereby incorporated by reference in its entirety. Said ASCII copy, created on Jul. 29, 2020, is named 167705_011501 PCT_SL.txt and is 98,534 bytes in size.

### BACKGROUND OF THE INVENTION

Most dominant human mutations associated with disease are single nucleotide substitutions. While gene editing strategies (e.g., CRISPR/Cas) are being developed to correct such mutations, single nucleotide discrimination and editing can be difficult to achieve for several reasons. For example, commonly used endonucleases, such as Streptococcus pyogenes Cas9 (SpCas9), can tolerate up to seven mismatches between a guide RNA (gRNA) and a nucleic acid molecule. Furthermore, the protospacer-adjacent motif (PAM) in some Cas9 enzymes can tolerate mismatches with the target DNA. There is a need for improved gene editing compositions capable of selectively and efficiently targeting a mutant allele, but not targeting a wild-type allele.

### SUMMARY OF THE INVENTION

As described below, the present invention features a Cas9 variant capable of selectively targeting a mutant allele without disrupting the wildtype allele, and methods of using such Cas9 variants to disrupt the mutant allele. In particular embodiments, the invention provides for the disruption of dominant mutations associated with single nucleotide substitutions. The invention further provides methods and compositions for treating a disease or condition or symptoms thereof associated with a dominant mutation (e.g., DFN36).

In one aspect, a method is provided for allele specific gene editing, the method includes contacting a double stranded polynucleotide having a mutant allele with a guide RNA that binds the mutant allele and a Cas9 polypeptide with a PAM site selective for the mutant allele, such that indels are selectively induced in the mutant target allele.

Another aspect of the invention provides a method for the allele-specific disruption of a dominant mutation, the method comprising contacting a double stranded polynucleotide comprising a mutant allele with a guide RNA that binds the mutant allele and a Cas9 nuclease with a PAM site selective for the mutant allele, such that indels are selectively induced in the mutant allele.

In one embodiment of the methods described above, the double stranded polynucleotide is DNA. In an embodiment, the DNA is genomic DNA. In another embodiment, the polynucleotide is present in a cell. In another embodiment, the cell is a cell in vivo or in vitro.

In another aspect, a method is provided for the treatment of a disorder associated with a dominant mutant allele in a target gene, the method includes contacting a cell heterozygous for the dominant mutant allele in a target gene with a guide RNA that binds the target mutant allele and a Cas9 nuclease with a PAM site selective for the mutant allele and selectively inducing an indel in the mutant target allele.

Another aspect provides a method of treating progressive hearing loss in a subject, the method includes contacting a cell of a subject heterozygous for a p.M418K mutation in TMC1 with a SaCas9-KKH and a guide RNA that targets TMC1 and inducing indels in the TMC1 allele having the p.M418K mutation, thereby treating hearing loss in the subject

Another aspect of the present disclosure includes a vector having a polynucleotide encoding a SaCas9-KKH polypeptide, or a fragment thereof, and a gRNA having a nucleic acid sequence complementary to a nucleic acid sequence comprising a mutation associated with DFNA36. In one embodiment, the vector is included in a pharmaceutical composition.

In various embodiments of any of the above aspects, the cell is a cell of the inner ear In various embodiments of any of the above aspects, the cell is an inner or outer hair cell. In various embodiments of any of the above aspects, the administering improves or maintains auditory function in the subject. In various embodiments of any of the above aspects, an improvement in auditory function is associated with preservation of hair bundle morphology and/or restoration of mechanotransduction. In various embodiments of any of the above aspects, the guide RNA and the Cas9 polypeptide are encoded in a single vector. In various embodiments of any of the above aspects, the vector is an adeno-associated virus vector or a lentivirus vector. In various embodiments of any of the above aspects, the contacting includes transfecting cells in the subject with a guide RNA and a polynucleotide encoding a Cas9 protein. In various embodiments of any of the above aspects, the guide RNA and the Cas9 polypeptide are administered simultaneously.

Other features and advantages of the invention will be apparent from the detailed description and from the claims.

### Definitions

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise.

By “AAV9-php.b vector” is meant a viral vector comprising an AAV9-php.b polynucleotide or fragment thereof that transfects a cell of the inner ear. In one embodiment, the AAV9-php.b vector transfects at least 70% of inner hair cells and 70% of outer hair cells following administration to the inner ear of a subject or contact with a cell derived from an inner ear in vitro. In other embodiments, at least 85%, 90%, 95% or virtually 100% of inner hair cells and/or 85%, 90%, 95% or virtually 100% of outer hair cells are transfected. The transfection efficiency may be assessed using a gene encoding GFP in a mouse model. The sequence of an exemplary AAV9-php.b vector is provided below.

AAV9-php.b
CCAATGATACGCGTCGGTGCGGGCCTCTTCGCTATTACGCCAGCTGGCGAAAGGGGGATGTGCTGCAAGGCGATT
AAGTTGGGTAACGCCAGGGTTTTCCCAGTCACGACGTTGTAAAACGACGGCCAGTGAGCGCGCGTAATACGACTC
ACTATAGGGCGAATTGGGTACATCGACGGTATCGGGGGAGCTCGCAGGGTCTCCATTTTGAAGCGGGAGGTTTGA
ACGCGCAGCCGCCATGCCGGGGTTTTACGAGATTGTGATTAAGGTCCCCAGCGACCTTGACGAGCATCTGCCCGG
CATTTCTGACAGCTTTGTGAACTGGGTGGCCGAGAAGGAATGGGAGTTGCCGCCAGATTCTGACATGGATCTGAA
TCTGATTGAGCAGGCACCCCTGACCGTGGCCGAGAAGCTGCAGCGCGACTTTCTGACGGAATGGCGCCGTGTGAG
TAAGGCCCCGGAGGCTCTTTTCTTTGTGCAATTTGAGAAGGGAGAGAGCTACTTCCACATGCACGTGCTCGTGGA
AACCACCGGGGTGAAATCCATGGTTTTGGGACGTTTCCTGAGTCAGATTCGCGAAAAACTGATTCAGAGAATTTA
CCGCGGGATCGAGCCGACTTTGCCAAACTGGTTCGCGGTCACAAAGACCAGAAATGGCGCCGGAGGCGGGAACAA
GGTGGTGGATGAGTGCTACATCCCCAATTACTTGCTCCCCAAAACCCAGCCTGAGCTCCAGTGGGCGTGGACTAA
TATGGAACAGTATTTAAGCGCCTGTTTGAATCTCACGGAGCGTAAACGGTTGGTGGCGCAGCATCTGACGCACGT
GTCGCAGACGCAGGAGCAGAACAAAGAGAATCAGAATCCCAATTCTGATGCGCCGGTGATCAGATCAAAAACTTC
AGCCAGGTACATGGAGCTGGTCGGGTGGCTCGTGGACAAGGGGATTACCTCGGAGAAGCAGTGGATCCAGGAGGA
CCAGGCCTCATACATCTCCTTCAATGCGGCCTCCAACTCGCGGTCCCAAATCAAGGCTGCCTTGGACAATGCGGG
AAAGATTATGAGCCTGACTAAAACCGCCCCCGACTACCTGGTGGGCCAGCAGCCCGTGGAGGACATTTCCAGCAA
TCGGATTTATAAAATTTTGGAACTAAACGGGTACGATCCCCAATATGCGGCTTCCGTCTTTCTGGGATGGGCCAC
GAAAAAGTTCGGCAAGAGGAACACCATCTGGCTGTTTGGGCCTGCAACTACCGGGAAGACCAACATCGCGGAGGC
CATAGCCCACACTGTGCCCTTCTACGGGTGCGTAAACTGGACCAATGAGAACTTTCCCTTCAACGACTGTGTGGA
CAAGATGGTGATCTGGTGGGAGGAGGGGAAGATGACCGCCAAGGTCGTGGAGTCGGCCAAAGCCATTCTCGGAGG
AAGCAAGGTGCGCGTGGACCAGAAATGCAAGTCCTCGGCCCAGATAGACCCGACTCCCGTGATCGTCACCTCCAA
CACCAATATGTGCGCCGTGATTGACGGGAACTCAACGACCTTCGAACACCAGCAGCCGTTGCAAGACCGGATGTT
CAAATTTGAACTCACCCGCCGTCTGGATCATGACTTTGGGAAGGTCACCAAGCAGGAAGTCAAAGACTTTTTCCG
GTGGGCAAAGGATCACGTGGTTGAGGTGGAGCATGAATTCTACGTCAAAAAGGGTGGAGCCAAGAAAAGACCCGC
CCCCAGTGACGCAGATATAAGTGAGCCCAAACGGGTGCGCGAGTCAGTTGCGCAGCCATCGACGTCAGACGCGGA
AGCTTCGATCAACTACGCGGACAGGTACCAAAACAAATGTTCTCGTCACGTGGGCATGAATCTGATGCTGTTTCC
CTGCAGACAATGCGAGAGACTGAATCAGAATTCAAATATCTGCTTCACTCACGGTGTCAAAGACTGTTTAGAGTG
CTTTCCCGTGTCAGAATCTCAACCCGTTTCTGTCGTCAAAAAGGCGTATCAGAAACTGTGCTACATTCATCACAT
CATGGGAAAGGTGCCAGACGCTTGCACTGCTTGCGACCTGGTCAATGTGGACTTGGATGACTGTGTTTCTGAACA
ATAAATGACTTAAACCAGGTATGAGTCGGCTGGATAAATCTAAAGTCATAAACGGCGCTCTGGAATTACTCAATG
AAGTCGGTATCGAAGGCCTGACGACAAGGAAACTCGCTCAAAAGCTGGGAGTTGAGCAGCCTACCCTGTACTGGC
ACGTGAAGAACAAGCGGGCCCTGCTCGATGCCCTGGCCATCGAGATGCTGGACAGGCATCATACCCACTTCTGCC
CCCTGGAAGGCGAGTCATGGCAAGACTTTCTGCGGAACAACGCCAAGTCATTCCGCTGTGCTCTCCTCTCACATC
GCGACGGGGCTAAAGTGCATCTCGGCACCCGCCCAACAGAGAAACAGTACGAAACCCTGGAAAATCAGCTCGCGT
TCCTGTGTCAGCAAGGCTTCTCCCTGGAGAACGCACTGTACGCTCTGTCCGCCGTGGGCCACTTTACACTGGGCT
GCGTATTGGAGGAACAGGAGCATCAAGTAGCAAAAGAGGAAAGAGAGACACCTACCACCGATTCTATGCCCCCAC
TTCTGAGACAAGCAATTGAGCTGTTCGACCGGCAGGGAGCCGAACCTGCCTTCCTTTTCGGCCTGGAACTAATCA
TATGTGGCCTGGAGAAACAGCTAAAGTGCGAAAGCGGCGGGCCGGCCGACGCCCTTGACGATTTTGACTTAGACA
TGCTCCCAGCCGATGCCCTTGACGACTTTGACCTTGATATGCTGCCTGCTGACGCTCTTGACGATTTTGACCTTG
ACATGCTCCCCGGGTAAATGCATGAATTCGATCTAGAGGGCCCTATTCTATAGTGTCACCTAAATGCTAGAGCTC
GCTGATCAGCCTCGACTGTGCCTTCTAGTTGCCAGCCATCTGTTGTTTGCCCCTCCCCCGTGCCTTCCTTGACCC
TGGAAGGTGCCACTCCCACTGTCCTTTCCTAATAAAATGAGGAAATTGCATCGCATTGTCTGAGTAGGTGTCATT
CTATTCTGGGGGGTGGGGTGGGGCAGGACAGCAAGGGGGAGGATTGGGAAGACAATAGCAGGCATGCTGGGGATG
CGGTGGGCTCTATGGCTTCTGAGGCGGAAAGAACCAGCTGGGGCTCGAATCAAGCTATCAAGTGCCACCTGACGT
CTCCCTATCAGTGATAGAGAAGTCGACACGTCTCGAGCTCCCTATCAGTGATAGAGAAGGTACGTCTAGAACGTC
TCCCTATCAGTGATAGAGAAGTCGACACGTCTCGAGCTCCCTATCAGTGATAGAGAAGGTACGTCTAGAACGTCT
CCCTATCAGTGATAGAGAAGTCGACACGTCTCGAGCTCCCTATCAGTGATAGAGAAGGTACGTCTAGAACGTCTC
CCTATCAGTGATAGAGAAGTCGACACGTCTCGAGCTCCCTATCAGTGATAGAGAAGGTACCCCCTATATAAGCAG
AGAGATCTGTTCAAATTTGAACTGACTAAGCGGCTCCCGCCAGATTTTGGCAAGATTACTAAGCAGGAAGTCAAG
GACTTTTTTGCTTGGGCAAAGGTCAATCAGGTGCCGGTGACTCACGAGTTTAAAGTTCCCAGGGAATTGGCGGGA
ACTAAAGGGGCGGAGAAATCTCTAAAACGCCCACTGGGTGACGTCACCAATACTAGCTATAAAAGTCTGGAGAAG
CGGGCCAGGCTCTCATTTGTTCCCGAGACGCCTCGCAGTTCAGACGTGACTGTTGATCCCGCTCCTCTGCGACCG
CTAGCTTCGATCAACTACGCAGACAGGTACCAAAACAAGTGTTCTCGTCACGTGGGCATTAATCTGATTCTGTTT
CCCTGCAGACAATGCGAGAGAATGAATCAGAACTCAAATATCTGCTTCACTCACGGACAGAAAGACTGTTTAGAG
TGCTTTCCCGTGTCAGAATCTCAACCCGTTTCTGTCGTCAAAAAGGCGTATCAGAAACTGTGCTACATTCATCAT
ATCATGGGAAAGGTGCCAGACGCTTGCACTGCCTGCGATCTGGTCAATGTGGATTTGGATGACTGCATCTTTGAA
CAATAAATGACTTAAGCCAGGTATGGCTGCCGATGGTTATCTTCCAGATTGGCTCGAGGACAACCTTAGTGAAGG
AATTCGCGAGTGGTGGGCTTTGAAACCTGGAGCCCCTCAACCCAAGGCAAATCAACAACATCAAGACAACGCTAG
AGGTCTTGTGCTTCCGGGTTACAAATACCTTGGACCCGGCAACGGACTCGACAAGGGGGAGCCGGTCAACGCAGC
AGACGCGGCGGCCCTCGAGCACGACAAAGCCTACGACCAGCAGCTCAAGGCCGGAGACAACCCGTACCTCAAGTA
CAACCACGCCGACGCCGAGTTCCAGGAGCGGCTCAAAGAAGATACGTCTTTTGGGGGCAACCTCGGGCGAGCAGT
CTTCCAGGCCAAAAAGAGGCTTCTTGAACCTCTTGGTCTGGTTGAGGAAGCGGCTAAGACGGCTCCTGGAAAGAA
GAGGCCTGTAGAGCAGTCTCCTCAGGAACCGGACTCCTCCGCGGGTATTGGCAAATCGGGTGCACAGCCCGCTAA
AAAGAGACTCAATTTCGGTCAGACTGGCGACACAGAGTCAGTCCCAGACCCTCAACCAATCGGAGAACCTCCCGC
AGCCCCCTCAGGTGTGGGATCTCTTACAATGGCTTCAGGTGGTGGCGCACCAGTGGCAGACAATAACGAAGGTGC
CGATGGAGTGGGTAGTTCCTCGGGAAATTGGCATTGCGATTCCCAATGGCTGGGGGACAGAGTCATCACCACCAG
CACCCGAACCTGGGCCCTGCCCACCTACAACAATCACCTCTACAAGCAAATCTCCAACAGCACATCTGGAGGATC
TTCAAATGACAACGCCTACTTCGGCTACAGCACCCCCTGGGGGTATTTTGACTTCAACAGATTCCACTGCCACTT
CTCACCACGTGACTGGCAGCGACTCATCAACAACAACTGGGGATTCCGGCCTAAGCGACTCAACTTCAAGCTCTT
TAACATTCAGGTCAAAGAGGTTACGGACAACAATGGAGTCAAGACCATCGCCAATAACCTTACCAGCACGGTCCA
GGTCTTCACGGACTCAGACTATCAGCTCCCGTACGTGCTCGGGTCGGCTCACGAGGGCTGCCTCCCGCCGTTCCC
AGCGGACGTTTTCATGATTCCTCAGTACGGGTATCTGACGCTTAATGATGGAAGCCAGGCCGTGGGTCGTTCGTC
CTTTTACTGCCTGGAATATTTCCCGTCGCAAATGCTAAGAACGGGTAACAACTTCCAGTTCAGCTACGAGTTTGA
GAACGTACCTTTCCATAGCAGCTACGCTCACAGCCAAAGCCTGGACCGACTAATGAATCCACTCATCGACCAATA
CTTGTACTATCTCTCTAGAACTATTAACGGTTCTGGACAGAATCAACAAACGCTAAAATTCAGTGTGGCCGGACC
CAGCAACATGGCTGTCCAGGGAAGAAACTACATACCTGGACCCAGCTACCGACAACAACGTGTCTCAACCACTGT
GACTCAAAACAACAACAGCGAATTTGCTTGGCCTGGAGCTTCTTCTTGGGCTCTCAATGGACGTAATAGCTTGAT
GAATCCTGGACCTGCTATGGCCTCTCACAAAGAAGGAGAGGACCGTTTCTTTCCTTTGTCTGGATCTTTAATTTT
TGGCAAACAAGGTACTGGCAGAGACAACGTGGATGCGGACAAAGTCATGATAACCAACGAAGAAGAAATTAAAAC
TACTAACCCGGTAGCAACGGAGTCCTATGGACAAGTGGCCACAAACCACCAGAGTGCCCAAACTTTGGCGGTGCC
TTTTAAGGCACAGGCGCAGACCGGTTGGGTTCAAAACCAAGGAATACTTCCGGGTATGGTTTGGCAGGACAGAGA
TGTGTACCTGCAAGGACCCATTTGGGCCAAAATTCCTCACACGGACGGCAACTTTCACCCTTCTCCGCTGATGGG
AGGGTTTGGAATGAAGCACCCGCCTCCTCAGATCCTCATCAAAAACACACCTGTACCTGCGGATCCTCCAACGGC
CTTCAACAAGGACAAGCTGAACTCTTTCATCACCCAGTATTCTACTGGTCAAGTCAGCGTGGAGATCGAGTGGGA
GCTGCAGAAGGAAAACAGCAAGCGCTGGAACCCGGAGATCCAGTACACTTCCAACTATTACAAGTCTAATAATGT
TGAATTTGCTGTTAATACTGAAGGTGTATATAGTGAACCCCGCCCCATTGGCACCAGATACCTGACTCGTAATCT
GTAAGTCGACTTGCTTGTTAATCAATAAACCGTTTAATTCGTTTCAGTTGAACTTTGGTCTCTGCGAAGGGCAAT
TCGTTTAAACCTGCAGGACTAGAGGTCCTGTATTAGAGGTCACGTGAGTGTTTTGCGACATTTTGCGACACCATG
TGGTCACGCTGGGTATTTAAGCCCGAGTGAGCACGCAGGGTCTCCATTTTGAAGCGGGAGGTTTGAACGCGCAGC
CGCCAAGCCGAATTCTGCAGATATCACATGTCCTAGGAACTATCGATCCATCACACTGGCGGCCGCTCGACTAGA
GCGGCCGCCACCGCGGTGGAGCTCCAGCTTTTGCGGACCGAATCGGAAAGAACATGTGAGCAAAAGGCCAGCAAA
AGGCCAGGAACCGTAAAAAGGCCGCGTTGCTGGCGTTTTTCCATAGGCTCCGCCCCCCTGACGAGCATCACAAAA
ATCGACGCTCAAGTCAGAGGTGGCGAAACCCGACAGGACTATAAAGATACCAGGCGTTTCCCCCTGGAAGCTCCC
TCGTGCGCTCTCCTGTTCCGACCCTGCCGCTTACCGGATACCTGTCCGCCTTTCTCCCTTCGGGAAGCGTGGCGC
TTTCTCATAGCTCACGCTGTAGGTATCTCAGTTCGGTGTAGGTCGTTCGCTCCAAGCTGGGCTGTGTGCACGAAC
CCCCCGTTCAGCCCGACCGCTGCGCCTTATCCGGTAACTATCGTCTTGAGTCCAACCCGGTAAGACACGACTTAT
CGCCACTGGCAGCAGCCACTGGTAACAGGATTAGCAGAGCGAGGTATGTAGGCGGTGCTACAGAGTTCTTGAAGT
GGTGGCCTAACTACGGCTACACTAGAAGAACAGTATTTGGTATCTGCGCTCTGCTGAAGCCAGTTACCTTCGGAA
AAAGAGTTGGTAGCTCTTGATCCGGCAAACAAACCACCGCTGGTAGCGGTGGTTTTTTTGTTTGCAAGCAGCAGA
TTACGCGCAGAAAAAAAGGATCTCAAGAAGATCCTTTGATCTTTTCTACGGGGTCTGACGCTCAGTGGAACGAAA
ACTCACGTTAAGGGATTTTGGTCATGAGATTATCAAAAAGGATCTTCACCTAGATCCTTTTAAATTAAAAATGAA
GTTTTAAATCAATCTAAAGTATATATGAGTAAACTTGGTCTGACAGTTACCAATGCTTAATCAGTGAGGCACCTA
TCTCAGCGATCTGTCTATTTCGTTCATCCATAGTTGCCTGACTCCCCGTCGTGTAGATAACTACGATACGGGAGG
GCTTACCATCTGGCCCCAGTGCTGCAATGATACCGCGAGACCCACGCTCACCGGCTCCAGATTTATCAGCAATAA
ACCAGCCAGCCGGAAGGGCCGAGCGCAGAAGTGGTCCTGCAACTTTATCCGCCTCCATCCAGTCTATTAATTGTT
GCCGGGAAGCTAGAGTAAGTAGTTCGCCAGTTAATAGTTTGCGCAACGTTGTTGCCATTGCTACAGGCATCGTGG
TGTCACGCTCGTCGTTTGGTATGGCTTCATTCAGCTCCGGTTCCCAACGATCAAGGCGAGTTACATGATCCCCCA
TGTTGTGCAAAAAAGCGGTTAGCTCCTTCGGTCCTCCGATCGTTGTCAGAAGTAAGTTGGCCGCAGTGTTATCAC
TCATGGTTATGGCAGCACTGCATAATTCTCTTACTGTCATGCCATCCGTAAGATGCTTTTCTGTGACTGGTGAGT
ACTCAACCAAGTCATTCTGAGAATAGTGTATGCGGCGACCGAGTTGCTCTTGCCCGGCGTCAATACGGGATAATA
CCGCGCCACATAGCAGAACTTTAAAAGTGCTCATCATTGGAAAACGTTCTTCGGGGCGAAAACTCTCAAGGATCT
TACCGCTGTTGAGATCCAGTTCGATGTAACCCACTCGTGCACCCAACTGATCTTCAGCATCTTTTACTTTCACCA
GCGTTTCTGGGTGAGCAAAAACAGGAAGGCAAAATGCCGCAAAAAAGGGAATAAGGGCGACACGGAAATGTTGAA
TACTCATACTCTTCCTTTTTCAATATTATTGAAGCATTTATCAGGGTTATTGTCTCATGAGCGGATACATATTTG
AATGTATTTAGAAAAATAAACAAATAGGGGTTCCGCGCACATTTCCCCGAAAAGTGCCACCTGACGTC

By “administer” is meant providing one or more compositions described herein to a subject. By way of example and without limitation, composition administration can be performed by injection, for example, into the cochlea. Other routes that deliver the composition to cells affected by a mutation can be employed. Administration can be, for example, by bolus injection or by gradual perfusion over time.

By “agent” is meant any small molecule chemical compound, antibody, nucleic acid molecule, or polypeptide, or fragments thereof.

By “alteration” is meant a change (increase or decrease) in the expression levels or activity of a gene or polypeptide as detected by standard art known methods such as those described herein. As used herein, an alteration includes a 10% change in expression levels, preferably a 25% change, more preferably a 40% change, and most preferably a 50% or greater change in expression levels.

By “ameliorate” is meant decrease, suppress, attenuate, diminish, arrest, or stabilize the development or progression of a disease. Exemplary diseases include any disease associated with a dominant mutation. In one embodiment, the disease is hearing loss associated with a dominant mutation, for example, Deafness, Deafness, Autosomal Dominant 3B, Deafness, Autosomal Dominant 9, Autosomal Dominant 11, Deafness, Autosomal Dominant 12, Deafness, Autosomal Dominant 13, Deafness, Autosomal Dominant17, Deafness, Autosomal Dominant 20, Deafness, Autosomal Dominant 22, Deafness, Autosomal Dominant 25, Deafness, Autosomal Dominant 36, Deafness, Autosomal Dominant 41, Deafness, Autosomal Dominant 66, Deafness, Autosomal Dominant 68, Deafness, and Autosomal Dominant Nonsyndromic Sensorineural 39, with Dentinogenesis Imperfecta 1.

By “Anc80 polypeptide” is meant a capsid polypeptide having at least about 85% amino acid identity to the following polypeptide sequence:

MAADGYLPDWLEDNLSEGIREWWDLKPGAPKPKANQQKQDDGRGLVLPGY
KYLGPFNGLDKGEPVNAADAAALEHDKAYDQQLKAGDNPYLRYNHADAEF
QERLQEDTSFGGNLGRAVFQAKKRVLEPLGLVEEGAKTAPGKKRPVEQSP
QEPDSSSGIGKKGQQPARKRLNFGQTGDSESVPDPQPLGEPPAAPSGVGS
NTMAAGGGAPMADNNEGADGVGNASGNWHCDSTWLGDRVITTSTRT ALP
TYNNHLYKQISSQSGGSTNDNTYFGYSTPWGYFDFNRFHCHFSPRDWQRL
INNNWGFRPKKLNFKLFNIQVKEVTTNDGTTTIANNLTSTVQVFTDSEYQ
LPYVLGSAHQGCLPPFPADVFMIPQYGYLTLNNGSQAVGRSSFYCLEYFP
SQMLRTGNNFQFSYTFEDVPFHSSYAHSQSLDRLMNPLIDQYLYYLSRTQ
TTSGTAGNRTLQFSQAGPSSMANQAKNWLPGPCYRQQRVSKTTNQNNNSN
FAWTGATKYHLNGRDSLVNPGPAMATHKDDEDKFFPMSGVLIFGKQGAGN
SNVDLDNVMITNEEEIKTTNPVATEEYGTVATNLQSANTAPATGTVNSQG
ALPGMVWQDRDVYLQGPIWAKIPHTDGHFHPSPLMGGFGLKHPPPQILIK
NTPVPANPPTTFSPAKFASFITQYSTGQVSVEIE ELQKENSKRWNPEIQ
YTSNYNKSTNVDFAVDTNGVYSEPRPIGTRYLTRNL

By “Anc80 polynucleotide” is meant a nucleic acid molecule encoding a Anc80 polypeptide.

By “Cas9 (CRISPR associated protein 9)” is meant a polypeptide or fragment thereof having at least about 85% amino acid identity to NCBI Accession No. NP_269215 and having RNA binding activity, DNA binding activity, and/or DNA cleavage activity (e.g., endonuclease or nickase activity). An exemplary Cas9 polypeptide sequence is provided below.

| | |
|---|---|
| 1 | mdkkysigld igtnsvgwav itdeykvpsk kfkvlgntdr hsikknliga llfdsgetae |
| 61 | atrlkrtarr rytrrknric ylqeifsnem akvddsffhr leesflveed kkherhpifg |
| 121 | nivdevayhe kyptiyhlrk klvdstdkad lrliylalah mikfrghfli egdlnpdnsd |
| 181 | vdklfiqlvq tynqlfeenp inasgvdaka ilsarlsksr rlenliaqlp gekknglfgn |
| 241 | lialslgltp nfksnfdlae daklqlskdt ydddldnlla qigdqyadlf laaknlsdai |
| 301 | llsdilrvnt eitkaplsas mikrydehhq dltllkalvr qqlpekykei ffdqskngya |
| 361 | gyidggasqe efykfikpil ekmdgteell vklnredllr kqrtfdngsi phqihlgelh |
| 421 | ailrrqedfy pflkdnreki ekiltfripy yvgplargns rfawmtrkse etitpwnfee |
| 481 | vvdkgasaqs fiermtnfdk nlpnekvlpk hsllyeyftv yneltkvkyv tegmrkpafl |
| 541 | sgeqkkaivd llfktnrkvt vkqlkedyfk kiecfdsvei sgvedrfnas lgtyhdllki |
| 601 | ikdkdfldne enedilediv ltltlfedre mieerlktya hlfddkvmkq lkrrrytgwg |
| 661 | rlsrklingi rdkqsgktil dflksdgfan rnfmqlihdd sltfkediqk aqvsgqgdsl |
| 721 | hehianlags paikkgilqt vkvvdelvkv mgrhkpeniv iemarenqtt qkgqknsrer |
| 781 | mkrieegike lgsqilkehp ventqlqnek lylyylqngr dmyvdgeldi nrlsdydvdh |
| 841 | ivpqsflkdd sidnkvltrs dknrgksdnv pseevvkkmk nywrqllnak litqrkfdnl |
| 901 | tkaergglse ldkagfikrq lvetrqitkh vaqildsrmn tkydendkli revkvitlks |
| 961 | klvsdfrkdf qfykvreinn yhhandayln avvgtalikk ypklesefvy gdykvydvrk |
| 1021 | miakseqeig katakyffys nimnffktei tlangeirkr plietngetg eivwdkgrdf |
| 1081 | atvrkvlsmp qvnivkktev qtggfskesi 1pkrnsdkli arkkdwdpkk yggfdsptva |
| 1141 | ysvlvvakve kgkskklksv kellgitime rssfeknpid fleakgykev kkdliiklpk |
| 1201 | yslfelengr krmlasagel qkgnelalps kyvnflylas hyeklkgspe dneqkqlfve |
| 1261 | qhkhyldeii eqisefskry iladanldkv lsaynkhrdk pireqaenii hlftltnlga |
| 1321 | paafkyfdtt idrkrytstk evldatlihq sitglyetri dlsqlggd |

By “Cas 9 nucleic acid molecule” is meant a polynucleotide encoding a Cas9 polypeptide or fragment thereof. An exemplary Cas9 nucleic acid molecule sequence is provided at NCBI Accession No. NC_002737 and is shown below.

| | |
|---|---|
| 1 | atggataaga aatactcaat aggcttagat atcggcacaa atagcgtcgg atgggcggtg |
| 61 | atcactgatg aatataaggt tccgtctaaa aagttcaagg ttctgggaaa tacagaccgc |
| 121 | cacagtatca aaaaaaatct tataggggct cttttatttg acagtggaga gacagcggaa |
| 181 | gcgactcgtc tcaaacggac agctcgtaga aggtatacac gtcggaagaa tcgtatttgt |
| 241 | tatctacagg agattttttc aaatgagatg gcgaaagtag atgatagttt ctttcatcga |
| 301 | cttgaagagt cttttttggt ggaagaagac aagaagcatg aacgtcatcc tatttttgga |
| 361 | aatatagtag atgaagttgc ttatcatgag aaatatccaa ctatctatca tctgcgaaaa |
| 421 | aaattggtag attctactga taaagcggat ttgcgcttaa tctatttggc cttagcgcat |
| 481 | atgattaagt ttcgtggtca ttttttgatt gagggagatt taaatcctga taatagtgat |
| 541 | gtggacaaac tatttatcca gttggtacaa acctacaatc aattatttga agaaaaccct |
| 601 | attaacgcaa gtggagtaga tgctaaagcg attctttctg cacgattgag taaatcaaga |
| 661 | cgattagaaa atctcattgc tcagctcccc ggtgagaaga aaaatggctt atttgggaat |
| 721 | ctcattgctt tgtcattggg tttgacccct aattttaaat caaattttga tttggcagaa |
| 781 | gatgctaaat tacagctttc aaaagatact tacgatgatg atttagataa tttattggcg |
| 841 | caaattggag atcaatatgc tgatttgttt ttggcagcta agaatttatc agatgctatt |
| 901 | ttactttcag atatcctaag agtaaatact gaaataacta aggctcccct atcagcttca |
| 961 | atgattaaac gctacgatga acatcatcaa gacttgactc ttttaaaagc tttagttcga |
| 1021 | caacaacttc cagaaaagta taaagaaatc ttttttgatc aatcaaaaaa cggatatgca |
| 1081 | ggttatattg atgggggagc tagccaagaa gaattttata aatttatcaa accaatttta |
| 1141 | gaaaaaatgg atggtactga ggaattattg gtgaaactaa atcgtgaaga tttgctgcgc |
| 1201 | aagcaacgga cctttgacaa cggctctatt ccccatcaaa ttcacttggg tgagctgcat |
| 1261 | gctattttga gaagacaaga agacttttat ccatttttaa aagacaatcg tgagaagatt |
| 1321 | gaaaaaatct tgacttttcg aattccttat tatgttggtc cattggcgcg tggcaatagt |
| 1381 | cgttttgcat ggatgactcg gaagtctgaa gaaacaatta ccccatggaa ttttgaagaa |
| 1441 | gttgtcgata aaggtgcttc agctcaatca tttattgaac gcatgacaaa ctttgataaa |
| 1501 | aatcttccaa atgaaaaagt actaccaaaa catagtttgc tttatgagta ttttacggtt |
| 1561 | tataacgaat tgacaaaggt caaatatgtt actgaaggaa tgcgaaaacc agcatttctt |
| 1621 | tcaggtgaac agaagaaagc cattgttgat ttactcttca aaacaaatcg aaaagtaacc |
| 1681 | gttaagcaat taaaagaaga ttatttcaaa aaaatagaat gttttgatag tgttgaaatt |
| 1741 | tcaggagttg aagatagatt taatgcttca ttaggtacct accatgattt gctaaaaatt |
| 1801 | attaaagata aagatttttt ggataatgaa gaaaatgaag atatcttaga ggatattgtt |
| 1861 | ttaacattga ccttatttga agatagggag atgattgagg aaagacttaa aacatatgct |
| 1921 | cacctctttg atgataaggt gatgaaacag cttaaacgtc gccgttatac tggttgggga |
| 1981 | cgtttgtctc gaaaattgat taatggtatt agggataagc aatctggcaa aacaatatta |
| 2041 | gattttttga aatcagatgg ttttgccaat cgcaatttta tgcagctgat ccatgatgat |
| 2101 | agtttgacat ttaaagaaga cattcaaaaa gcacaagtgt ctggacaagg cgatagttta |
| 2161 | catgaacata ttgcaaattt agctggtagc cctgctatta aaaaaggtat tttacagact |
| 2221 | gtaaaagttg ttgatgaatt ggtcaaagta atggggcggc ataagccaga aaatatcgtt |
| 2281 | attgaaatgg cacgtgaaaa tcagacaact caaaagggcc agaaaaattc gcgagagcgt |
| 2341 | atgaaacgaa tcgaagaagg tatcaaagaa ttaggaagtc agattcttaa agagcatcct |
| 2401 | gttgaaaata ctcaattgca aaatgaaaag ctctatctct attatctcca aaatggaaga |
| 2461 | gacatgtatg tggaccaaga attagatatt aatcgtttaa gtgattatga tgtcgatcac |
| 2521 | attgttccac aaagtttcct taaagacgat tcaatagaca ataaggtctt aacgcgttct |
| 2581 | gataaaaatc gtggtaaatc ggataacgtt ccaagtgaag aagtagtcaa aaagatgaaa |
| 2641 | aactattgga gacaacttct aaacgccaag ttaatcactc aacgtaagtt tgataattta |
| 2701 | acgaaagctg aacgtggagg tttgagtgaa cttgataaag ctggttttat caaacgccaa |
| 2761 | ttggttgaaa ctcgccaaat cactaagcat gtggcacaaa ttttggatag tcgcatgaat |
| 2821 | actaaatacg atgaaaatga taaacttatt cgagaggtta aagtgattac cttaaaatct |
| 2881 | aaattagttt ctgacttccg aaaagatttc caattctata aagtacgtga gattaacaat |
| 2941 | taccatcatg cccatgatgc gtatctaaat gccgtcgttg gaactgcttt gattaagaaa |
| 3001 | tatccaaaac ttgaatcgga gtttgtctat ggtgattata aagtttatga tgttcgtaaa |
| 3061 | atgattgcta agtctgagca agaaataggc aaagcaaccg caaaatattt cttttactct |
| 3121 | aatatcatga acttcttcaa aacagaaatt acacttgcaa atggagagat tcgcaaacgc |
| 3181 | cctctaatcg aaactaatgg ggaaactgga gaaattgtct gggataaagg gcgagatttt |
| 3241 | gccacagtgc gcaaagtatt gtccatgccc caagtcaata ttgtcaagaa aacagaagta |
| 3301 | cagacaggcg gattctccaa ggagtcaatt ttaccaaaaa gaaattcgga caagcttatt |
| 3361 | gctcgtaaaa aagactggga tccaaaaaaa tatggtggtt ttgatagtcc aacggtagct |
| 3421 | tattcagtcc tagtggttgc taaggtggaa aaagggaaat cgaagaagtt aaaatccgtt |
| 3481 | aaagagttac tagggatcac aattatggaa agaagttcct ttgaaaaaaa tccgattgac |
| 3541 | tttttagaag ctaaaggata taaggaagtt aaaaaagact taatcattaa actacctaaa |
| 3601 | tatagtcttt ttgagttaga aaacggtcgt aaacggatgc tggctagtgc cggagaatta |
| 3661 | caaaaaggaa atgagctggc tctgccaagc aaatatgtga attttttata tttagctagt |
| 3721 | cattatgaaa agttgaaggg tagtccagaa gataacgaac aaaaacaatt gtttgtggag |
| 3781 | cagcataagc attatttaga tgagattatt gagcaaatca gtgaattttc taagcgtgtt |
| 3841 | attttagcag atgccaattt agataaagtt cttagtgcat ataacaaaca tagagacaaa |
| 3901 | ccaatacgtg aacaagcaga aaatattatt catttattta cgttgacgaa tcttggagct |
| 3961 | cccgctgctt ttaaatattt tgatacaaca attgatcgta aacgatatac gtctacaaaa |
| 4021 | gaagttttag atgccactct tatccatcaa tccatcactg gtctttatga aacacgcatt |
| 4081 | gatttgagtc agctaggagg tgactga |

SaCas9:
MAPKKKRKVGIHGVPAAKRNYILGLDIGITSVGYGIIDYETRDVIDAGVRLFKEANVENNEGRRSKRGARRL
KRRRRHRIQRVKKLLFDYNLLTDHSELSGINPYEARVKGLSQKLSEEEFSAALLHLAKRRGVHNVNEVEEDT
GNELSTKEQISRNSKALEEKYVAELQLERLKKDGEVRGSINRFKTSDYVKEAKQLLKVQKAYHQLDQSFIDT
YIDLLETRRTYYEGPGEGSPFGWKDIKEWYEMLMGHCTYFPEELRSVKYAYNADLYNALNDLNNLVITRDEN
EKLEYYEKFQIIENVFKQKKKPTLKQIAKEILVNEEDIKGYRVTSTGKPEFTNLKVYHDIKDITARKEIIEN
AELLDQIAKILTIYQSSEDIQEELTNLNSELTQEEIEQISNLKGYTGTHNLSLKAINLILDELWHTNDNQIA
IFNRLKLVPKKVDLSQQKEIPTTLVDDFILSPVVKRSFIQSIKVINAIIKKYGLPNDIIIELAREKNSKDAQ
KMINEMQKRNRQTNERIEEIIRTTGKENAKYLIEKIKLHDMQEGKCLYSLEAIPLEDLLNNPFNYEVDHIIP
RSVSFDNSFNNKVLVKQEENSKKGNRTPFQYLSSSDSKISYETFKKHILNLAKGKGRISKTKKEYLLEERDI
NRFSVQKDFINRNLVDTRYATRGLMNLLRSYFRVNNLDVKVKSINGGFTSFLRRKWKFKKERNKGYKHHAED
ALIIANADFIFKEWKKLDKAKKVMENQMFEEKQAESMPEIETEQEYKEIFITPHQIKHIKDFKDYKYSHRVD
KKPNRELINDTLYSTRKDDKGNTLIVNNLNGLYDKDNDKLKKLINKSPEKLLMYHHDPQTYQKLKLIMEQYG
DEKNPLYKYYEETGNYLTKYSKKDNGPVIKKIKYYGNKLNAHLDITDDYPNSRNKVVKLSLKPYRFDVYLDN
GVYKFVTVKNLDVIKKENYYEVNSKCYEEAKKLKKISNQAEFIASFYNNDLIKINGELYRVIGVNNDLLNRI
EVNMIDITYREYLENMNDKRPPRIIKTIASKTQSIKKYSTDILGNLYEVKSKKHPQIIKKGKRPAATKKAGQ
AKKKKGS
SaCas9-KKH
Bold and underlined denotes variation from SaCas9
MAPKKKRKVGIHGVPAAKRNYILGLDIGITSVGYGIIDYETRDVIDAGVRLFKEANVENNEGRRSKRGARRL
KRRRRHRIQRVKKLLFDYNLLTDHSELSGINPYEARVKGLSQKLSEEEFSAALLHLAKRRGVHNVNEVEEDT
GNELSTKEQISRNSKALEEKYVAELQLERLKKDGEVRGSINRFKTSDYVKEAKQLLKVQKAYHQLDQSFIDT
YIDLLETRRTYYEGPGEGSPFGWKDIKEWYEMLMGHCTYFPEELRSVKYAYNADLYNALNDLNNLVITRDEN
EKLEYYEKFQIIENVFKQKKKPTLKQIAKEILVNEEDIKGYRVTSTGKPEFTNLKVYHDIKDITARKEIIEN
AELLDQIAKILTIYQSSEDIQEELTNLNSELTQEEIEQISNLKGYTGTHNLSLKAINLILDELWHTNDNQIA
IFNRLKLVPKKVDLSQQKEIPTTLVDDFILSPVVKRSFIQSIKVINAIIKKYGLPNDIIIELAREKNSKDAQ
KMINEMQKRNRQTNERIEEIIRTTGKENAKYLIEKIKLHDMQEGKCLYSLEAIPLEDLLNNPFNYEVDHIIP
RSVSFDNSFNNKVLVKQEENSKKGNRTPFQYLSSSDSKISYETFKKHILNLAKGKGRISKTKKEYLLEERDI
NRFSVQKDFINRNLVDTRYATRGLMNLLRSYFRVNNLDVKVKSINGGFTSFLRRKWKFKKERNKGYKHHAED
ALIIANADFIFKEWKKLDKAKKVMENQMFEEKQAESMPEIETEQEYKEIFITPHQIKHIKDFKDYKYSHRVD
KKPNRKLINDTLYSTRKDDKGNTLIVNNLNGLYDKDNDKLKKLINKSPEKLLMYHHDPQTYQKLKLIMEQYG
DEKNPLYKYYEETGNYLTKYSKKDNGPVIKKIKYYGNKLNAHLDITDDYPNSRNKVVKLSLKPYRFDVYLDN
GVYKFVTVKNLDVIKKENYYEVNSKCYEEAKKLKKISNQAEFIASFYKNDLIKINGELYRVIGVNNDLLNRI
EVNMIDITYREYLENMNDKRPPHIIKTIASKTQSIKKYSTDILGNLYEVKSKKHPQIIKKGKRPAATKKAGQ
AKKKKGS

Kleinstiver et al. (Nat Biotechnol. 2015 December; 33(12):1293-1298. doi: 10.1038/nbt.3404. Epub 2015 Nov. 2) describes SaCas9-KKH.

“Detect” refers to identifying the presence, absence or amount of the analyte to be detected.

By “detectable label” is meant a composition that when linked to a molecule of interest renders the latter detectable, via spectroscopic, photochemical, biochemical, immunochemical, or chemical means. For example, useful labels include radioactive isotopes, magnetic beads, metallic beads, colloidal particles, fluorescent dyes, electron-dense reagents, enzymes (for example, as commonly used in an ELISA), biotin, digoxigenin, or haptens.

By “disease” is meant any condition or disorder that damages or interferes with the normal function of a cell, tissue, or organ. Examples of diseases include any pathology, such as a hearing disorder, associated with a dominant mutation.

By “effective amount” is meant the amount of an agent required to ameliorate the symptoms of a disease relative to an untreated patient. The effective amount of active compound(s) used to practice the present invention for therapeutic treatment of a disease varies depending upon the manner of administration, the age, body weight, and general health of the subject. Ultimately, the attending physician or veterinarian will decide the appropriate amount and dosage regimen. Such amount is referred to as an “effective” amount.

By “fragment” is meant a portion of a polypeptide or nucleic acid molecule. This portion contains, preferably, at least 10%, 20%, 30%, 40%, 50%, 60%, 70%, 80%, or 90% of the entire length of the reference nucleic acid molecule or polypeptide. A fragment may contain 10, 20, 30, 40, 50, 60, 70, 80, 90, or 100, 200, 300, 400, 500, 600, 700, 800, 900, or 1000 nucleotides or amino acids.

“Hybridization” means hydrogen bonding, which may be Watson-Crick, Hoogsteen or reversed Hoogsteen hydrogen bonding, between complementary nucleobases. For example, adenine and thymine are complementary nucleobases that pair through the formation of hydrogen bonds.

By “identity” is meant the amino acid or nucleic acid sequence identity between a sequence of interest and a reference sequence. Sequence identity is typically measured using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine. In an exemplary approach to determining the degree of identity, a BLAST program may be used, with a probability score between e⁻³ and e⁻¹⁰⁰ indicating a closely related sequence.

By “identity” is meant the amino acid or nucleic acid sequence identity between a sequence of interest and a reference sequence. Sequence identity is typically measured using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine. In an exemplary approach to determining the degree of identity, a BLAST program may be used, with a probability score between e⁻³ and e⁻¹⁰⁰ indicating a closely related sequence.

The term “indel” refers to the insertion or deletion of at least one nucleotide at a locus in a nucleic acid molecule. An indel present in the coding region of a gene may result in a frameshift mutation resulting in a premature stop codon or other signal for the expressed protein to be degraded.

The terms “isolated,” “purified,” or “biologically pure” refer to material that is free to varying degrees from components which normally accompany it as found in its native state. “Isolate” denotes a degree of separation from original source or surroundings. “Purify” denotes a degree of separation that is higher than isolation. A “purified” or “biologically pure” protein is sufficiently free of other materials such that any impurities do not materially affect the biological properties of the protein or cause other adverse consequences. That is, a nucleic acid or peptide of this invention is purified if it is substantially free of cellular material, viral material, or culture medium when produced by recombinant DNA techniques, or chemical precursors or other chemicals when chemically synthesized. Purity and homogeneity are typically determined using analytical chemistry techniques, for example, polyacrylamide gel electrophoresis or high-performance liquid chromatography. The term “purified” can denote that a nucleic acid or protein gives rise to essentially one band in an electrophoretic gel. For a protein that can be subjected to modifications, for example, phosphorylation or glycosylation, different modifications may give rise to different isolated proteins, which can be separately purified.

By “isolated polynucleotide” is meant a nucleic acid (e.g., a DNA) that is free of the genes which, in the naturally-occurring genome of the organism from which the nucleic acid molecule of the invention is derived, flank the gene. The term therefore includes, for example, a recombinant DNA that is incorporated into a vector; into an autonomously replicating plasmid or virus; or into the genomic DNA of a prokaryote or eukaryote; or that exists as a separate molecule (for example, a cDNA or a genomic or cDNA fragment produced by PCR or restriction endonuclease digestion) independent of other sequences. In addition, the term includes an RNA molecule that is transcribed from a DNA molecule, as well as a recombinant DNA that is part of a hybrid gene encoding additional polypeptide sequence.

By an “isolated polypeptide” is meant a polypeptide of the invention that has been separated from components that naturally accompany it. Typically, the polypeptide is isolated when it is at least 60%, by weight, free from the proteins and naturally-occurring organic molecules with which it is naturally associated. Preferably, the preparation is at least 75%, more preferably at least 90%, and most preferably at least 99%, by weight, a polypeptide of the invention. An isolated polypeptide of the invention may be obtained, for example, by extraction from a natural source, by expression of a recombinant nucleic acid encoding such a polypeptide; or by chemically synthesizing the protein. Purity can be measured by any appropriate method, for example, column chromatography, polyacrylamide gel electrophoresis, or by HPLC analysis.

By “marker” is meant any protein or polynucleotide having an alteration in expression level or activity that is associated with a disease or disorder.

By “mechanosensation” is meant a response to a mechanical stimulus. Touch, hearing, and balance of examples of the conversion of a mechanical stimulus into a neuronal signal. Mechanosensory input is converted into a response to a mechanical stimulus through a process termed “mechanotransduction.”

As used herein, “obtaining” as in “obtaining an agent” includes synthesizing, purchasing, or otherwise acquiring the agent.

As used herein, the terms “prevent,” “preventing,” “prevention,” “prophylactic treatment” and the like refer to reducing the probability of developing a disorder or condition in a subject, who does not have, but is at risk of or susceptible to developing a disorder or condition.

By “promoter” is meant a polynucleotide sufficient to direct transcription of a downstream polynucleotide.

By “Espin promoter” is meant a regulatory polynucleotide sequence derived from NCBI Reference Sequence: NG_015866.1 that is sufficient to direct expression of a downstream polynucleotide in an outer or inner hair cell, vestibular hair cell, a spiral ganglion, or a vestibular ganglion. In one embodiment, the Espin promoter comprises or consists of at least about 350, 500, 1000, 2000, 3000, 4000, 5000, or more base pairs upstream of an Espin coding sequence.

By “protocadherin related 15 (PCDH15) promoter” is meant a regulatory polynucleotide sequence derived from NCBI Reference Sequence: NG_009191 that is sufficient to direct expression of a downstream polynucleotide in an outer or inner hair cell, vestibular hair cell, a spiral ganglion, or a vestibular ganglion. In one embodiment, the PCDH15 promoter comprises at least about 350, 500, 1000, 2000, 3000, 4000, 5000, or more base pairs upstream of an PCDH15 coding sequence. In some embodiments, the PCDH15 promoter comprises or consists of a nucleic acid sequence having at least about 85% sequence identity to the following nucleotide sequence:

TCTTCACCTGTCATTTTCAACCAGCCTCAGCCTATCTGCTCTGTCACAAT
CACTACTAAAATATGTTCCTAAATTGCTTGTTTCTAGATCCTTCCTTCTC
ATATGCTCAGGTGAACACATGGGTGAAATTTAATATGGAATTGAAATATG
TACTATGCAAGATAGATTCCTTAAGAAATGTTTCTCTGATTTATATGACA
TAATTGTATTTTACTAGTTTACCTGTCCATCTGTAAAACTTTGTTTTGGA
GATTTCATATATTACAATGTTTAAGAAATATGCTATAATGTTTTGTATAG
TATATTTCTTCGTGATAACCTTATATACTACCAGTCACACGTGTTTGTAA
AAATCTAAAGAGTACTTTTGGCTCCTACAGAATGTGTGAAGTTGTGAAAT
TGTTTTTTTGTTTTGTTTTGTTTTGTTTTTATGCCCCAAAGATGTGGAGG
GCTTCATATAAGAGGGTAGATTTAATGAGAGAGAGAGGGAGAGACAGAGA
GAATGATAAAAGAAGCTTAAGAGATTATTTTATCTTGTCAACGACATTGT
TATTGAATGTAAGCTGCTAAACTTCTTAGATAAAGTAAAACAGTAAAAAC
AAACACACAAAACAGAACAGAGAATCATCAGACAGGCTGACGAACACAGT
ACAATAAAGCAGCCAGTACCGATGATCAGTGGACATCAATTTGTCTTTTG
GGCTGTAGCACCTGCTACTAATTGGTGCAAAGCGCTCACCAGTCAGTGCG
TGGTTTAGCGCACTCAGCTGTCTCCTGTATGTGCTGCGAGAAGCAAGATA
GCTAATTGCTGTTGCTTCAGTGCCAGTGAAATCAACGTGCTGAGCTAATA
GCGACAGATAGAGGGCAGACAGATTCCTGCTAGCAGCTTAGTGTTAGTTG
CTTGTGGTAACTAAGGCAGGTGGCATACATCTCAGAACGTGGAGAATGAT
GGTATGCTTTCTGA

By “protein tyrosine phosphatase, receptor type Q (PTPRQ) promoter” is meant a regulatory polynucleotide sequence derived from GeneID: 374462 that is sufficient to direct expression of a downstream polynucleotide in an outer or inner hair cell, vestibular hair cell, a spiral ganglion, or a vestibular ganglion. In one embodiment, the PTPRQ promoter comprises at least about 350, 500, 1000, 2000, 3000, 4000, 5000, or more base pairs upstream of an PTPRQ coding sequence. In some embodiments, the PTPRQ promoter comprises or consists of a nucleic acid sequence having at least about 85% sequence identity to the following nucleotide sequence:

TGGTAGCCTCCCTAGAGACACAGAGCTGGGCCGGATGAGTCCAGGCACTG
ACGTGATCCATTATCTTTCACCTTAAAGAGTAAAAGGGAAACTAAAGTTA
ATTACCTCCACGAAACAAAAAGGTGCCTTCTTGTGCTTCAATTACATGGA
TATATTCTACTAGTCTAAAAGTATCTTCTCACTTCTTTCTGTCACTGTGA
GGACTTGAGTCAGAAGAAAGTTTAAATACAGTCATTGAGCTGGAAAGAGT
GGAAAGAGAAGCAAAGAGGGGGAAGCTGTAGGAAGGACGAAGTCACCCCC
AAGATACATGGTTACTGCTTACACCAAGCAAGCTGCCTTGGGAACGCTTC
CCCCGAGCAGCCAGAATGCTCAGCAGTGGAAGACACCTCTATTCCTGTAG
GCGAGTCCTGGGAAGCTGGTCAATCTGCAAATGCCAATTCCCAGCAGTGA
GCTCGGTCCACGTGTAAATCAAGATTTGGGGAAAGAGTAGGGTGGGTGGC
ATGGTTGACAATGTCATCAGCTCCCTCCTCTGACTCCTGTGGTCGTGCCC
CCATCTACTCTCACTCAGCTACACCCCACCTTCGGATTTGTGATGGACGC
TGGGTCCCTAGTAACCACAGCAAGTGTCTCCCCCGCACTTCCCCCTTCCC
CACCCCCACCCCCACCCCCAACCACCACCCCAGCGATGGAGCCTACTCTG
CTCCAAGCCGCCGCTAAGACCCGGAGAAGCGGAATTTCACTTTGAAATTC
CCTTGCCTCGTGAGGGCCGGCGCTGGGCATGCTCAGTAGCCGCGGCGCTG
CTGCTGGGCTGCTGGGCTGGCGCGGAGTCCACCCTGCCGTCTCCGCCTTG
GCTTCTGGGCGTCCAGAAGGCCAGGCATTTGCCGCCTCTGAGCGCTTCTG
TTCCCCTTACCCGCAACCTCCTACTGCTCTTCCTCTCTCCCTCTCTTAGG
GAGGTTGAAGCTGGTGCTGGTTTCTGTCGGCGCCACAGACTGACTGCTCT
GCAAACCCCAGCCGAGGACCTGAATCCCGGAGACTAGAAG

By “lipoma HMGIC fusion partner-like 5 (LHFPL5) promoter” also termed “TMHS promoter” is meant a regulatory polynucleotide sequence derived from NCBI Reference Sequence: GeneID: 222662 that is sufficient to direct expression of a downstream polynucleotide in an outer or inner hair cell, vestibular hair cell, a spiral ganglion, or a vestibular ganglion. In one embodiment, the TMHS promoter comprises at least about 350, 500, 1000, 2000, 3000, 4000, 5000, or more base pairs upstream of an PCDH15 coding sequence. In some embodiments, the TMHS promoter comprises or consists of a nucleic acid sequence having at least about 85% sequence identity to the following nucleotide sequence:

GCCCAGTGGAATTTTCCTAGTTCTTTACACTAGCCATGTATTTACCTATA
AAATCAGGAGAAATATGTATATATATAATATATTAAAACATATATATATT
TAAATGGGGAAATATGTAACAAACAAATAGAAACAAGGGGAGAAAGGCAT
TGTATTTGACAAAACACATATGTTCAGGTCTGAGAAGGCTCATAAAGAAT
GTTGTCTGCTATACTTTGTAGTTGCTTCTGTTATCACACAATCAGTCTGC
ATATACAGGCGTTTTATATATATATTTATATAGACTACATATATACGTAT
ATTATATATGTAAATATTTCACTGTCTTTGAGGACGGGGGCCCTGTCTTT
TTTATCTGTGGTTTTGCTTAGATGTCCTCCAACATAATCTTAACACATAG
TATGCTTTTAGAAATCGTTGACTGAATGCTAAGGACGAAAAACCGGTGAC
CAGAAGGCAACCAGGAAAGGCTTTGCTGACCTCCGGAGTGGTGGAGTTGG
AGGTTCTGGGAAGGCGACTAGGGAGCCAGGCAGGGGCGGGGTGGGATGGG
ATGTGGACAGCGCTTTTGCGGGGGGAAAGCGTTTTTGCTGCTGGAATTGA
GCAGTAGGAATGTGTCAGTCACATCCCCACCTTCCCAATTCTTGTCATCT
CGGTTCAGGAAGGTGAACGGTGTTCCGATTCCCCGCGGCGGGGGCCTGTA
GTGGGAGCTCTGCCCCTTCCCCGCCTCTGCTGCAGGCCCCGCCCCTCGCC
CGGAACCCCGGGGCGCTGGCCGCGGTGCTGAAACGGCGCCCTCCGCGGAC
GGAGGAGGGGGCGGGGCTCTCGGGAGCCGTGAGCCGGGAAGAGGGAGACG
GGCAGGGCGGCGCCAGCAGGCCCTGGTGGGCTTGGGAGGAGGCAGGAGAC
TGGAGACAGCCTCGGCTAGAGCGGACACAGGCACCTGGCAAGCTTTCCTT
GACCAAATCAAGGT

By “synapsin promoter” also termed “Syn promoter” is meant a regulatory polynucleotide sequence comprising or consisting of a nucleic acid sequence sufficient to direct expression of a downstream polynucleotide in an outer or inner hair cell, a vestibular hair cell, a spiral ganglion, or a vestibular ganglion and having at least about 85% sequence identity to the following nucleotide sequence:

tctagactgcagagggccctgcgtatgagtgcaagtgggttttaggacca
ggatgaggcggggtgggggtgcctacctgacgaccgaccccgacccactg
gacaagcacccaacccccattccccaaattgcgcatcccctatcagagag
ggggaggggaaacaggatgcggcgaggcgcgtgcgcactgccagcttcag
caccgcggacagtgccttcgcccccgcctggcggcgcgcgccaccgccgc
ctcagcactgaaggcgcgctgacgtcactcgccggtcccccgcaaactcc
ccttcccggccaccttggtcgcgtccgcgccgccgccggcccagccggac
cgcaccacgcgaggcgcgagatagggggGcacgggcgcgaccatctgcgc
tgcggcgccggcgactcagcgctgcctcagtctgcggtgggcagcggagg
agtcgtgtcgtgcctgagagcgcagtc

By “reduces” is meant a negative alteration of at least 10%, 25%, 50%, 75%, or 100%.

By “reference” is meant a standard or control condition.

A “reference sequence” is a defined sequence used as a basis for sequence comparison. A reference sequence may be a subset of or the entirety of a specified sequence; for example, a segment of a full-length cDNA or gene sequence, or the complete cDNA or gene sequence. For polypeptides, the length of the reference polypeptide sequence will generally be at least about 16 amino acids, preferably at least about 20 amino acids, more preferably at least about 25 amino acids, and even more preferably about 35 amino acids, about 50 amino acids, or about 100 amino acids. For nucleic acids, the length of the reference nucleic acid sequence will generally be at least about 50 nucleotides, preferably at least about 60 nucleotides, more preferably at least about 75 nucleotides, and even more preferably about 100 nucleotides or about 300 nucleotides or any integer thereabout or therebetween.

By “specifically binds” is meant a compound or antibody that recognizes and binds a polypeptide of the invention, but which does not substantially recognize and bind other molecules in a sample, for example, a biological sample, which naturally includes a polypeptide of the invention.

Nucleic acid molecules useful in the methods of the invention include any nucleic acid molecule that encodes a polypeptide of the invention or a fragment thereof. Such nucleic acid molecules need not be 100% identical with an endogenous nucleic acid sequence, but will typically exhibit substantial identity. Polynucleotides having “substantial identity” to an endogenous sequence are typically capable of hybridizing with at least one strand of a double-stranded nucleic acid molecule. Nucleic acid molecules useful in the methods of the invention include any nucleic acid molecule that encodes a polypeptide of the invention or a fragment thereof. Such nucleic acid molecules need not be 100% identical with an endogenous nucleic acid sequence, but will typically exhibit substantial identity. Polynucleotides having “substantial identity” to an endogenous sequence are typically capable of hybridizing with at least one strand of a double-stranded nucleic acid molecule. By “hybridize” is meant pair to form a double-stranded molecule between complementary polynucleotide sequences (e.g., a gene described herein), or portions thereof, under various conditions of stringency. (See, e.g., Wahl, G. M. and S. L. Berger (1987) Methods Enzymol. 152:399; Kimmel, A. R. (1987) Methods Enzymol. 152:507).

For example, stringent salt concentration will ordinarily be less than about 750 mM NaCl and 75 mM trisodium citrate, preferably less than about 500 mM NaCl and 50 mM trisodium citrate, and more preferably less than about 250 mM NaCl and 25 mM trisodium citrate. Low stringency hybridization can be obtained in the absence of organic solvent, e.g., formamide, while high stringency hybridization can be obtained in the presence of at least about 35% formamide, and more preferably at least about 50% formamide. Stringent temperature conditions will ordinarily include temperatures of at least about 30° C., more preferably of at least about 37° C., and most preferably of at least about 42° C. Varying additional parameters, such as hybridization time, the concentration of detergent, e.g., sodium dodecyl sulfate (SDS), and the inclusion or exclusion of carrier DNA, are well known to those skilled in the art. Various levels of stringency are accomplished by combining these various conditions as needed. In a preferred: embodiment, hybridization will occur at 30° C. in 750 mM NaCl, 75 mM trisodium citrate, and 1% SDS. In a more preferred embodiment, hybridization will occur at 37° C. in 500 mM NaCl, 50 mM trisodium citrate, 1% SDS, 35% formamide, and 100 pg/m1 denatured salmon sperm DNA (ssDNA). In a most preferred embodiment, hybridization will occur at 42° C. in 250 mM NaCl, 25 mM trisodium citrate, 1% SDS, 50% formamide, and 200 μg/ml ssDNA. Useful variations on these conditions will be readily apparent to those skilled in the art.

For most applications, washing steps that follow hybridization will also vary in stringency. Wash stringency conditions can be defined by salt concentration and by temperature. As above, wash stringency can be increased by decreasing salt concentration or by increasing temperature. For example, stringent salt concentration for the wash steps will preferably be less than about 30 mM NaCl and 3 mM trisodium citrate, and most preferably less than about 15 mM NaCl and 1.5 mM trisodium citrate. Stringent temperature conditions for the wash steps will ordinarily include a temperature of at least about 25° C., more preferably of at least about 42° C., and even more preferably of at least about 68° C. In a preferred embodiment, wash steps will occur at 25° C. in 30 mM NaCl, 3 mM trisodium citrate, and 0.1% SDS. In a more preferred embodiment, wash steps will occur at 42 C in 15 mM NaCl, 1.5 mM trisodium citrate, and 0.1% SDS. In a more preferred embodiment, wash steps will occur at 68° C. in 15 mM NaCl, 1.5 mM trisodium citrate, and 0.1% SDS. Additional variations on these conditions will be readily apparent to those skilled in the art. Hybridization techniques are well known to those skilled in the art and are described, for example, in Benton and Davis (Science 196:180, 1977); Grunstein and Hogness (Proc. Natl.

Acad. Sci., USA 72:3961, 1975); Ausubel et al. (Current Protocols in Molecular Biology, Wiley Interscience, New York, 2001); Berger and Kimmel (Guide to Molecular Cloning Techniques, 1987, Academic Press, New York); and Sambrook et al., Molecular Cloning: A Laboratory Manual, Cold Spring Harbor Laboratory Press, New York.

By “substantially identical” is meant a polypeptide or nucleic acid molecule exhibiting at least 50% identity to a reference amino acid sequence (for example, any one of the amino acid sequences described herein) or nucleic acid sequence (for example, any one of the nucleic acid sequences described herein). Preferably, such a sequence is at least 60%, more preferably 80% or 85%, and more preferably 90%, 95% or even 99% identical at the amino acid level or nucleic acid to the sequence used for comparison.

Sequence identity is typically measured using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine. In an exemplary approach to determining the degree of identity, a BLAST program may be used, with a probability score between e⁻³ and e⁻¹⁰⁰ indicating a closely related sequence.

By “subject” is meant a mammal, including, but not limited to, a human or non-human mammal, such as a bovine, equine, canine, ovine, or feline.

By “TMC1 polypeptide” is meant a polypeptide having at least about 85% or greater amino acid sequence identity to NCBI Reference Sequence: NP_619636.2 or a fragment thereof having mechanotransduction channel activity. An exemplary amino acid sequence of TMC1 is provided below:

| | |
|---|---|
| 1 | mspkkvqikv eekedetees sseeeeeved klprreslrp krkrtrdvin eddpepeped |
| 61 | eetrkareke rrrrlkrgae eeeideeele rlkaeldekr qiiatvkckp wkmekkievl |
| 121 | keakkfvsen egalgkgkgk rwfafkmmma kkwakflrdf enfkaacvpw enkikaiesq |
| 181 | fgssvasyfl flrwmygvnm vlfiltfsli mlpeylwglp ygslprktvp raeeasaanf |
| 241 | gvlydfngla qysvlfygyy dnkrtigwmn frlplsyflv gimcigysfl vvlkamtkni |
| 301 | gddgggddnt fnfswkvfts wdylignpet adnkfnsitm nfkeaiteek aaqveenvhl |
| 361 | irflrflanf fvfltlggsg ylifwavkrs qefaqqdpdt lgwweknemn mvmsllgmfc |
| 421 | ptlfdlfael edyhplialk wllgrifall lgnlyvfila lmdeinnkie eeklvkanit |
| 481 | lweanmikay nasfsenstg ppffvhpadv prgpcwetmv gqefvrltvs dvlttyvtil |
| 541 | igdflracfv rfcnycwcwd leygypsyte fdisgnvlal ifnqgmiwmg sffapslpgi |
| 601 | nilrlhtsmy fqcwavmccn vpearvfkas rsnnfylgml llilflstmp vlymivslpp |
| 661 | sfdcgpfsgk nrmfeviget lehdfpswma kilrqlsnpg lviavilvmv laiyylnata |
| 721 | kgqkaanldl kkkmkmqale nkmrnkkmaa araaaaagrq |

By “TMC1 polynucleotide” is meant a polynucleotide encoding a TMC1 polypeptide. The sequence of an exemplary TMC1 polynucleotide is provided at NCBI Reference

Sequence: NM 138691.2, which is reproduced below:

| | |
|---|---|
| 1 | cagaaactat gagggcagaa cccagcaatc tgtgctttct ttcacaagcc ctccaggagt |
| 61 | tgctgaaatt taggaatcat tgccccaaaa agtggccctc ataatgatgc cagatgggat |
| 121 | cttactctgt tgcccaggct ggagtgcagt ggtgcgatct cggctctctg caacctccgc |
| 181 | ctcccaggtt caagtgattc tcctgcctcg gcctcctgag tagctgggat ttcaggccat |
| 241 | gaaagatcac tgttttagtc tgcgtggtgc agtggaacag atagacctcg gtttgaatct |
| 301 | cagctctact gtttactaga catgaaatgg ggaaatctaa aatgagatgc cagaagcctc |
| 361 | aaaaatggaa aaccccctgt gcttcacatc tgaaaatctc tgctgggggc agcaactttg |
| 421 | agcctgtggg gaaggaactg tccacgtgga gtggtctggt gaatgcttaa ggagctgcag |
| 481 | aagggaagtc cctctccaaa ctagccagcc actgagacct tctgacagga cacccccagg |
| 541 | atgtcaccca aaaaagtaca aatcaaagtg gaggaaaaag aagacgagac tgaggaaagc |
| 601 | tcaagtgaag aggaagagga ggtggaagat aagctacctc gaagagagag cttgagacca |
| 661 | aagaggaaac ggaccagaga tgttatcaat gaggatgacc cagaacctga accagaggat |
| 721 | gaagaaacaa ggaaggcaag agaaaaagag aggaggagga ggctaaagag aggagcagaa |
| 781 | gaagaagaaa ttgatgaaga ggaattggaa agattgaagg cagagttaga tgagaaaaga |
| 841 | caaataattg ctactgtcaa atgcaaacca tggaagatgg agaagaaaat tgaagttctc |
| 901 | aaggaggcaa aaaaatttgt gagtgaaaat gaaggggctc ttgggaaagg aaaaggaaaa |
| 961 | cggtggtttg catttaagat gatgatggcc aagaaatggg caaaattcct ccgtgatttt |
| 1021 | gagaacttca aagctgcgtg tgtcccatgg gaaaataaaa tcaaggctat tgaaagtcag |
| 1081 | tttggctcct cagtggcctc atacttcctc ttcttgagat ggatgtatgg agtcaatatg |
| 1141 | gttctcttta tcctgacatt tagcctcatc atgttgccag agtacctctg gggtttgcca |
| 1201 | tatggcagtt tacctaggaa aaccgttccc agagccgaag aggcatcggc agcaaacttt |
| 1261 | ggtgtgttgt acgacttcaa tggtttggca caatattccg ttctctttta tggctattat |
| 1321 | gacaataaac gaacaattgg atggatgaat ttcaggttgc cgctctccta ttttctagtg |
| 1381 | gggattatgt gcattggata cagctttctg gttgtcctca aagcaatgac caaaaacatt |
| 1441 | ggtgatgatg gaggtggaga tgacaacact ttcaatttca gctggaaggt ctttaccagc |
| 1501 | tgggactacc tgatcggcaa tcctgaaaca gcagacaaca aatttaattc tatcacaatg |
| 1561 | aactttaagg aagctatcac agaagaaaaa gcagcccaag tagaagaaaa cgtccacttg |
| 1621 | atcagattcc tgaggtttct ggctaacttc ttcgtgtttc taacacttgg agggagtgga |
| 1681 | tacctcatct tttgggctgt gaagcgatcc caggaatttg cacagcaaga tcctgacacc |
| 1741 | cttgggtggt gggaaaaaaa tgaaatgaac atggttatgt ccctcctagg gatgttctgt |
| 1801 | ccaacattgt ttgacttatt tgctgaatta gaagactacc atcctctcat cgctttgaaa |
| 1861 | tggctactgg gacgcatttt tgctcttctt ttaggcaatt tatacgtatt tattcttgca |
| 1921 | ttaatggatg agattaacaa caagattgaa gaggagaagc tagtaaaggc caatattacc |
| 1981 | ctttgggaag ccaatatgat caaggcctac aatgcatcat tctctgaaaa tagcactgga |
| 2041 | ccaccctttt ttgttcaccc tgcagatgta cctcgaggac cttgctggga aacaatggtg |
| 2101 | ggacaggagt ttgtgaggct gacagtctct gatgttctga ccacctacgt cacaatcctc |
| 2161 | attggggact ttctaagggc atgttttgtg aggttttgca attattgctg gtgctgggac |
| 2221 | ttggagtatg gatatccttc atacaccgaa ttcgacatca gtggcaacgt cctcgctctg |
| 2281 | atcttcaacc aaggcatgat ctggatgggc tccttctttg ctcccagcct cccaggcatc |
| 2341 | aatatccttc gactccatac atccatgtac ttccagtgct gggccgttat gtgctgcaat |
| 2401 | gttcctgagg ccagggtctt caaagcttcc agatcaaata acttctacct gggcatgcta |
| 2461 | ctgctcatcc tcttcctgtc cacaatgcct gtcttgtaca tgatcgtgtc cctcccacca |
| 2521 | tcttttgatt gtggtccatt cagtggcaaa aatagaatgt ttgaagtcat tggagagacc |
| 2581 | ctggagcacg atttcccaag ctggatggcg aagatcttga gacagctttc aaaccctggg |
| 2641 | ctggtcattg ctgtcatttt ggtgatggtt ttggccatct attatctcaa tgctactgcc |
| 2701 | aagggccaga aggcagcgaa tctggatctc aaaaagaaga tgaaaatgca agctttggag |
| 2761 | aacaaaatgc gaaacaagaa aatggcagct gcacgagcag ctgcagctgc tggtcgccag |
| 2821 | taataagtat cctgagagcc cagaaaaggt acactttgcc ttgctgttta aaagtaatgc |
| 2881 | aatatgtgaa cgcccagaga acaagcactg tggaactgct attttcctgt tctacccttg |
| 2941 | atggattttc aaggtcatgc tggccaatta aggcatcatc agtcctacct gagcaacaag |
| 3001 | aatctaaact ttattccaag tcagaaactg tttctgcaga gccactctct cccctgctcc |
| 3061 | atttcgtgac tttttttttt tttttaacaa attgagttta gaagtgagtg taatccagca |
| 3121 | atacagttta ctggtttagt tggtgggtta attaaaaaaa atttgctcat atgaactttc |
| 3181 | attttatatg tttcttttgc c |

As used herein, the terms “treat,” “treating,” “treatment,” and the like refer to reducing or ameliorating a disorder and/or symptoms associated therewith. It will be appreciated that, although not precluded, treating a disorder or condition does not require that the disorder, condition or symptoms associated therewith be completely eliminated.

Unless specifically stated or obvious from context, as used herein, the term “or” is understood to be inclusive. Unless specifically stated or obvious from context, as used herein, the terms “a”, “an”, and “the” are understood to be singular or plural.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

In this disclosure, “comprises,” “comprising,” “containing” and “having” and the like can have the meaning ascribed to them in U.S. Patent law and can mean “includes,” “including,” and the like; “consisting essentially of” or “consists essentially” likewise has the meaning ascribed in U.S. Patent law and the term is open-ended, allowing for the presence of more than that which is recited so long as basic or novel characteristics of that which is recited is not changed by the presence of more than that which is recited, but excludes prior art embodiments.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof.

Any compositions or methods provided herein can be combined with one or more of any of the other compositions and methods provided herein.

### BRIEF DESCRIPTION OF THE DRAWINGS

FIGS. 1A-1G illustrate targeting the Tmc1^{Bth} allele with high-fidelity SpCas9s and SaCas9-KKH. FIG. 1A is a sequence alignment illustrating gRNA design. Mutation site is enclosed. PAM sites are marked by underlined nucleotides. Mismatching nucleotides are encircled. SaCas9-KKH: KKH PAM variant of SaCas9,eSpCas9(1.1), HypaCas9 and SpCas9-HF1 are different high fidelity variants of SpCas9 (used in combination with 1.1 gRNA, as shown in FIG. 5A). FIG. 1B is a graph showing gene editing efficiency as measured by mean indel percentage in Tmc1^{Bth/WT} and Tmc1^{WT/WT} fibroblasts determined by targeted deep-sequencing and CRISPResso analysis. SpCas9 with 1.1, 2.1 and 2.4 gRNA represent the same conditions as in FIG. 5 (except that cells were not sorted for GFP expression); however, experiments were repeated to allow for head-to-head comparison with SaCas9-KKH and high fidelity SpCas9 enzymes. Cells were transfected on two different occasions (SpCas9 only and SpCas9+gRNA1.1 on four occasions) and genomic DNA from two independent biological samples on eachtransfection day were pooled for sequencing. Data for Tmc1^{Bth/WT} is presented to the left of data for TMC1^{WT/WT} for each condition. FIG. 1C is a graph showing gene editing efficiency as measured by allele-specific indel analysis of the samples from FIG. 1B in Tmc1^{Bth/WT} cells. Tmc1^{Bth} and Tmc1^{WT} reads were segregated using a Python script and indel percentages were analyzed for each allele. Thus, one condition represents indel formation in the same population of cells. The numbers above the graphs show specificity towards the Beethoven allele, expressed as percentages. Data for Bth reads is presented to the left of data for WT reads for each condition. FIG. 1D is a sequence alignment of the most abundant reads in the SaCas9-KKH+gRNA 4.2 treated cells, shown separately for Tmc1^{Bth} (top) and Tmc1 ^{WT} (bottom) reads. The CRISPR cut site is marked by black dashed line. Dashes represent deleted nucleotides. Nucleotides in bold are substitutions; however, these were not quantified as CRISPR actions. Sequences were aligned to Bth allele, thus in the bottom panel, WT sequence appears as a substitution (an A to T change). Mutation site is marked by an arrow. FIG. 1E includes graphs illustrating the indel profiles from SaCas9-KKH+gRNA-4.2 transfected Tmc1^{Bth/WT} fibroblasts. Tmc1Bth and Tmc1WTreads are plotted separately. Minus numbers represent deletions, plus numbers representinsertions. Sequences without indels (value=0) are omitted from the chart. FIG. 1F is a pie chart showing the percentage of indels that cause inframe and frame shift mutations (percentages are shown) in the coding sequence after SaCas9-KKH+gRNA 4.2 transfection. FIG. 1G is output from a GUIDE-Seq analysis on SaCas9-KKH+gRNA 4.2 transfected Tmc1^{Bth/WT} fibroblasts. Genomic DNA was pooled from three biological replicates for sequencing on one occasion. The number next to read is read count in the GUIDE-Seq assay.

FIGS. 2A-2H illustrate in vivo genome editing with SaCas9-KKH. FIG. 2A is a schematic of the cassette encoding SaCas9-KKH and a guide RNA inserted into an AAV vector used in the study; ITR denotes inverted terminal repeat, CMV denotes the cytomegalovirus promoter, U6 denotes sequencing primer: 5′-GACTATCATATGCTTACCGT-3′; and AAV-Anc80 denotes the vector backbone. FIG. 2B provides an experimental overview for in vivo studies (ABR denotes auditory brain response). FIG. 2C is a graph of gene editing efficiency determined by targeted deep sequencing of noninjected or AAV-SaCas9-KKH-gRNA-4.2 injected whole cochleas (mean ±S.D.). In non-injected animals, background indel frequencies ranged between 0.05 -0.06%. Every data point represents a unique sequencing reaction from pooled cochleas includes the number of cochleas each data point, number of independent sequencing reactions for non-injected: 5 (P7), 2 (P14), 2 (P27), injected: 5 (P7), 2 (P14), 2 (P42), 2 (P55) and 2 (P196 WT)). ANOVA was performed to describe overall difference (F=445.8, p<0.0001) followed by Tukey's post hoc test. FIG. 2D comprises two graphs of indel profiles from AAV-SaCas9-KKH-gRNA-4.2 injected Tmc1^{Bth/WT} animals. Tmc1^{Bth} and Tmc1^{WT} reads are plotted separately. Minus numbers represent deletions, plus numbers represent insertions. Sequences without indels (value=0) are omitted from the chart. FIG. 2E is a sequence alignment showing the most abundant reads in the AAV-SaCas9-KKH+gRNA 4.2 injected Tmc1^{Bth/WT} animals, shown separately for Tmc1^{Bth} (top) and Tmc1^{WT} (bottom) reads. Arrow denotes mutation site. FIG. 2F is a graph of AAV integration into the Tmc1^{Bth} and Tmc1^{WT} alleles in non-injected and injected Tmc1^{Bth/WT} animals at P14-P55. Mean±SD, number of independent experiments and sequencing reactions: 2 for non-injected and 6 for injected). FIG. 2G is a series of sequence alignments that illustrate indel profiles and read abundance at the mRNA level from AAV-SaCas9-KKH-gRNA-4.2 injected Tmc1^{Bth/WT} animals. Arrow denotes mutation site. FIG. 2H is a graph showing the relative read counts for Tmc1^{Bth} and Tmc1^{WT} representing mRNA in non-injected and injected animals. The dashed line represents an equal ratio (ratio=1). ANOVA, p=0.0001, Dunnett's multiple comparisons: p=0.0005 for injected (P42) vs non-injected (P42) and p=0.0001 for injected (P55) vs non-injected (P42)).

FIGS. 3A-3I illustrate the effects of SaCas9-KKH+sgRNA4.2 on inner ear function in Bth mice. FIG. 3A provides representative sensory transduction currents recorded at P14-16 from apical inner and outer hair cells of uninjected Tmc1^{Bth/Δ}; Tmc2^{Δ/Δ} mice (Bth, left) and those injected with AAV-SaCas9-KKH-sgRNA4.2 at P1-P2 (Bth+SaCas9, right). FIG. 3B provides plots showing the mean (±SEM) maximal current amplitudes, measured at P14-16 for uninjected inner hair cells (IHC) (n=19; top) and outer hair cells (OHC) (n=10; bottom) from uninjected Tmc1^{Bth/Δ}; Tmc2^{Δ/Δ} (left) and injected (right) mice; (inner hair cells: 124±118 pA, n=7, p=3.4 ×10⁻⁶; outer hair cells: 71±41 pA, n=12,p=8.1×10⁻⁷; unpaired two-tailed t-test). FIG. 3C shows families of ABR waveforms recorded at eight weeks from an uninjected Tmc1^{Bth/WT} mouse (left) and a Tmc1^{Bth/WT} mouse injected with AAV-SaCas9-KKH-gRNA-4.2 at P1 (right). Arrows denote indicate threshold traces. Scale bar applies to all traces. FIG. 3D includes three graphs showing ABR thresholds plotted as a function of frequency for ten injected Tmc1^{Bth/WT} mice (plot lines without error bars). Mean (±S.D.) ABR thresholds for uninjected Tmc1^{Bth/WT} (top plot line with error bars, n=8), and uninjected Tmc1^{WT/WT} control mice (bottom plot line with error bars) tested at 4 (n=6), 8 (n=12) and 12 (n=12) weeks. FIG. 3E is a graph showing DPOAE thresholds verses stimulus frequency at 12 weeks for eight injected Tmc1^{Bth/WT} mice (plot lines without error bars). Mean±S.D. DPOAE thresholds for uninjected Tmc1^{Bth/WT} (top plot line with error bars, n=9), and uninjected Tmc1^{WT/WT} control mice (bottom plot line with error bars, n=13). FIG. 3F is a plot showing the mean (±S.D.) ABR thresholds at 8 kHz verses age, from 4 to 40 weeks (WT: n=6, 12, 12, 6; Tmc1^{Bth/WT}: n=8, 8, 9, 9, 6; +SaCas9-KKH: n=8, 7, 7, 4, 4; one-way ANOVA, p=0.32). FIG. 3G is a set of representative confocal images of 100-μm cochlear sections harvested at 24 weeks from 8, 16, and 32 kHz regions of four uninjected (top), and six injected Tmc1^{Bth/WT} mice (bottom). Tissue was stained for MYO7A (red) and actin (green). FIG. 3H comprises two plots showing the mean (±S.D.) number of IHCs (left) and OHCs (right) per 100-μm section for four uninjected and six injected Tmc1^{Bth/WT} mice. FIG. 3I is a series of scanning electron microscope (SEM) images of the apical cochlear sensory epithelium showing hair bundle morphology for one uninjected Tmc1^{WT/WT} (left), one uninjected Tmc1^{Bth/WT} (middle), and one injected Tmc1^{Bth/WT} mouse (right).

FIGS. 4A-4D illustrate human dominant mutations targetable with SaCas9 and SaCas9-KKH, and allele-specific targeting of human DFNA36. FIG. 4A. is a sequence alignment illustrating gRNA design targeting a human DFNA36 allele. Mutation site is enclosed. PAM sites are marked by underlined nucleotides. Mismatching nucleotides are encircled. FIG. 4B is a graph showing genome editing efficiency (indel formation percentage) in haploid TMC1^{DFNA36} and TMC1^{WT} cells after transfection with SaCas9-KKH and H1, H2, H3 gRNA (mean±SD, 3 biological replicates sequenced independently). Control cells were transfected with GFP only. Data for Tmc1^{DFNA36} is presented to the left of data for TMC1^{WT} for each condition. FIG. 4C is an illustration of the types of indels in the case of H2 gRNA in TMC1^{DFNA36} and TMC1^{WT} cells (CRISPResso analysis, similar results were obtained from all gRNAs and all biological replicates). FIG. 4D is an illustration of all human dominant mutations in the ClinVar database (accessed 2019.03.25) and mutations targetable with SaCas9 and SaCas9-KKH

FIGS. 5A-5I illustrate targeting Tmc1^{Bth} with SpCas9. FIG. 5A is a sequence alignment illustrating gRNA design for SpCas9. Mutation site is highlighted in red. PAM sites are marked by green nucleotides. Mismatching nucleotides are shown in blue. The numbers or letters (e.g. 1.1) next to the PAM site represent gRNAs IDs. The gRNA 1.1 presently disclosed is identical to the Tmc1-mut3 gRNA in the study of Gao et al. Plasmids encoding SpCas9-2A-GFP, along with the different gRNAs, were transfected into fibroblasts. Four days after transfection, GFP-positive cells were sorted by FACS. FIG. 5B includes Sanger sequencing traces from Tmc1^{Bth/WT} or Tmc1^{WT/WT} mouse fibroblasts transfected with SpCas9-2A-GFP with or without gRNA 1.1. GFP expressing cells were sorted by FACS 4 days after transfection. The mutation site is marked by red arrow. Additional peaks appearing downstream (marked by black arrowheads) of the mutation site demonstrate sequence heterogeneity and thus, indel formation. Similar results were obtained by all gRNAs from two technical replicates (forward and reverse sequencing). Genome editing is apparent both in Tmc1^{Bth/WT} or Tmc1^{WT/WT} cells with SpCas9+gRNA 1.1. FIG. 5C includes Sanger sequencing data analyzed by TIDE. The control sample (SpCas9-2A-GFP only, black) and the genome edited sample (SpCas9-2A-GFP +gRNA 1.1, green) are overlaid. Downstream of the expected cut site (blue dashed line) the percentage of aberrant sequences was quantified in the region for decomposition. FIG. 5D is a graph of indel percentages (mean±standard deviation) in Tmc1^{Bth/WT} or Tmc1^{WT/WT} cells based on TIDE analysis. Cells were transfected in duplicates and two independent sequencing reactions (forward and reverse) were performed. No indel formation was observed in the case of 3.1, 3.2 and 3.3 gRNAs. gRNA 1.4 showed minimal, but specific genome editing on the Tmc1^{Bth/WT} cells. All the other gRNAs mediated efficient indel formation both in Tmc1^{Bth/WT} or Tmc1^{Bth/WT} cells. Data for Tmc1^{Bth/WT} is presented to the left of data for TMC1^{WT/WT} for each condition. FIG. 5E provides pie charts summarizing the targeted deep sequencing of control cells transfected with SpCas9-2A-GFP only cells, and cells transfected with SpCas9-2A-GFP and WT gRNA or SpCas9-2A-GFP and one of the three most specific gRNAs (1.1, 2.1 and 2.4) in Tmc1^{Bth/WT} (top) Tmc1^{WT/WT} (bottom) cells. Indels were quantified after segregating Tmc1^{Bth} and Tmc1^{WT} reads by CRISPResso (only insertions and deletions were quantified, substitutions were ignored). None of the gRNAs are specific to the Tmc1^{Bth} allele, and mediate efficient indel formation on the Tmc1^{WT} allele as well (light blue). Sequencing was performed one time from pooled cells, transfected in triplicates. Numbers in pie charts represent the percentage of reads. Specificity was defined as the indel percentage towards the targeted allele among total indels. The gRNA with the highest selectivity towards the Tmc1^{Bth} allele was gRNA 2.4. On the top row of pie chart, the left most pie chart shows WT=50% and Bth=49.9%. The following pie charts show clockwise WT, WT indel, Bth, Bth indel (WT=43.4, 45.6, 48.1, and 16.3, respectively). On the bottom row of pie charts, WT and WT indel are shown, with WT being the larger percentage. FIG. 5F includes sequence alignments of the most abundant reads in the SpCas9+gRNA 2.4 treated cells, shown separately for Tmc1^{Bth} (top) and Tmc1^{WT} (bottom) reads. The CRISPR cut site is marked by a black dashed line. Dashes represent deleted nucleotides. Insertions are shown with nucleotides in red squares. Nucleotides in bold are substitutions, however these were not quantified as CRISPR actions. Sequences were aligned to Bth allele, thus in the bottom panel, WT reads appear as having a substitution (a T to A change). Mutation site is marked by red arrow. Indel formation is evident in both Tmc1^{Bth} and Tmc1^{WT} reads. FIG. 5G includes graphs of indel profiles from SpCas9+gRNA 2.4 transfected Tmc1^{Bth/WT} fibroblasts. Tmc1^{Bth} and Tmc1^{WT} reads are plotted separately. Minus numbers represent deletions, plus numbers represent insertions. Sequences without indels (value=0) are omitted from the chart. The most common indel event is a single base deletion. FIG. 5H is a pie chart of indels causing in-frame vs. frame shift mutations (percentages are shown) in the coding sequence after SpCas9+gRNA 2.4 transfection. FIG. 5I provides the GUIDE-Seq analysis on SpCas9+gRNA 2.4 transfected Tmc1^{Bth/WT} fibroblasts. Genomic DNA was isolated from 3 biological replicates for sequencing on one occasion. Only one off-target site was identified. Numbers next to reads are read counts in the GUIDE-Seq assay.

FIG. 6 is a graph of the number of indels based on targeted deep sequencing data from Tmc1^{Bth/WT} and Tmc1^{WT/WT} cell lines treated with different Cas9+gRNA combinations (from FIG. 1B). Note that data points show non-normalized read counts. Cells were transfected on two different occasions (SpCas9 only and SpCas9+gRNA 1.1 on four occasions) and genomic DNA from two independent biological samples on each transfection day were pooled for sequencing. Indels in the SaCas9-KKH treated Tmc1^{WT/WT} lines are not different from the background (i.e. untreated samples). This method revealed high sensitivity, as the indel rates in CRISPR treated samples were 40-160-fold higher than the background indel rates observed in untreated samples. Data for Tmc1^{Bth/WT} is presented to the left of data for TMC1^{WT/WT} for each condition.

FIGS. 7A-7D are graphs illustrating single nucleotide substitutions after Cas9+gRNA treatment. Cells were transfected on two different occasions (SpCas9 only and SpCas930 gRNA 1.1 on four occasions) and genomic DNA from two independent biological samples on each transfection day were pooled for sequencing. Experimental conditions are the same as in FIG. 1B. FIG. 7A is a graph illustrating substitutions given as percentages (i.e. normalized to total read counts). Analysis was performed on non-segregated .fastq files in Tmc1^{Bth/WT} cells and in Tmc1^{WT/WT} cells. FIG. 7B is a graph of substitutions given as non-normalized values (i.e. the number of reads with substitutions). Analysis was performed on non-segregated .fastq files in Tmc1^{Bth/WT} cells and in Tmc1^{WT/WT} cells . FIG. 7C is a graph illustrating substitutions given as percentages (i.e. normalized to total read counts). Analysis was performed on segregated .fastq files in Tmc1^{Bth/WT} cells. FIG. 7D is a graph of substitutions given as non-normalized values (i.e. the number of reads with substitutions). Analysis was performed on segregated .fastq files in Tmc1^{Bth/WT} cells. Substitutions were not frequent (0.1-0.5% of reads) and there was no difference between untreated and treated samples in the percentage or number of reads with single nucleotide substitutions. For FIGS. 7A-7D Data for Tmc1^{Bth/WT} is presented to the left of data for Tmc1^{WT/WT} for each condition.

FIG. 8 is a graph illustrating the background sequencing error and indel formation with SaCas9-KKH (mean±SD). Background sequencing error rate (GFP only) and comparison indel events in SaCas9-KKH+gRNA 4.1/gRNA 4.2 and gRNA 4.3 transfected Tmc1^{WT/WT} fibroblasts are plotted.

FIG. 9A-9G show the effects of SaCas9-KKH+sgRNA4.2 on inner ear function in WT & Bth mice. FIG. 9A. provides plots of the representative sensory transduction currents recorded at P14-16 from inner hair cells (IHCs) and outer hair cells (OHC) of wild-type (WT) mice injected with AAV-SaCas9-KKH-sgRNA4.2 at P1-P2. FIG. 9B is a graph showing the mean±SEM maximal transduction current amplitudes for P14-16 inner hair cells (left, n=8) and outer hair cells (right, n=6) from WT mice injected with AAV-SaCas9-KKH-sgRNA4.2 at P1-P2. FIG. 9C comprises plots of families of ABR waveforms recorded at eight weeks from an uninjected Tmc1^{WT/WT} mouse (left) and a Tmc1^{WT/WT} mouse injected with AAV-SaCas9-KKH-gRNA-4.2 at P1 (right). Bolded traces indicate threshold. Scale bar applies to all traces. FIG. 9D is a graph showing the mean±S.D. ABR thresholds plotted as a function of stimulus frequency for sixTmc1^{WT/WT} mice (black, n=6) and three Tmc1^{WT/WT} mice injected with AAV-SaCas9-KKH-gRNA-4.2 (gray) at 24 weeks of age (p=0.9). FIG. 9E is a graph showing the mean±S.D. ABR thresholds plotted as a function of stimulus frequency for six Tmc1^{WT/WT} mice (bottom plot line with error bars), nine Tmc1^{Bth/WT} (top plot line with error bars), and five Tmc1^{Bth/WT} mice injected with AAV-SaCas9-KKH-gRNA-4.2 (plot lines without error bars) at 24 weeks of age. 8-kHz thresholds for injected: 38±11 dB (n=9) and uninjected: 64±19 dB (n=8) were significantly different (p=0.004, unpaired two-tailed t-test). ABR thresholds at higher frequencies (22 kHz) for injected (84±15 dB, n=9) and uninjected Tmc1^{Bth/WT} mice (103 ±5 dB, n=8; p=0.004, unpaired two-tailed t-test). FIG. 9F is a series of representative confocal images of 100-μm cochlear sections harvested at 24 weeks from the 8, 16, and 32 kHz regions from three uninjected Tmc1^{WT/WT} mice (top), and three Tmc1^{WT/WT} mice injected with AAV-SaCas9-KKH-gRNA-4.2 (bottom). The tissue was stained for MYO7A (red) and actin (green). FIG. 9G comprises plots of the Mean±S.D. number of surviving inner and outer hair cells per 100-μm section (n=3).

FIG. 10A-10C illustrates ABR amplitude, latencies, and correlation of thresholds with surviving hair cells. FIG. 10A is a graph of Peak 1 amplitudes measured from 8 kHz ABR waveforms at the 8-week time point (from examples shown in FIGS. 9A and 9C) for all Tmc1^{Bth/WT} mice injected with AAV-SaCas9-KKH-gRNA-4.2 (all traces except trace denoted by arrow) and an example of an uninjected Tmc1^{Bth/WT} (arrow). FIG. 10B is a graph of Peak 1 latencies measured from 8 kHz ABR waveforms at the 8-week time point (from examples shown in FIGS. 9A and 9C, for all Tmc1^{Bth/WT} mice injected with AAV-SaCas9-KKH-gRNA-4.2 (all traces except trace denoted by arrow) and an example of an uninjected Tmc1^{Bth/WT} (arrow). FIG. 10C is a graph of the mean ABR thresholds measured at 24 weeks, evoked by 8 and 16 kHz tone bursts (from FIG. 9E) plotted as function of the mean percentage of surviving inner and outer hair cells from the 8 and 16 kHz regions (from FIG. 3H). The data were fit with a linear equation that had a slope of -2.1 dB/% and a correlation coefficient of -0.82 (line, Pearson's r).

FIG. 11 is a graph illustrating the results of qPCR specific for the inverted terminal repeat (ITR) region in the transfected plasmid (AAV-CMV-SaCas9-KKH-U6-gRNA) normalized to albumin gene in HAP-1DFNA36 and HAP1WT cells. Bars show mean±S.D. Data points are from three independent biological replicates. Data for Tmc1^{Bth/WT} is presented to the left of data for Tmc1^{WT/WT} for each condition.

FIG. 12 is a graph showing the number of targeted deep sequencing reads with indels from TMC1^{DFNA36} and TMC1^{WT} cells treated with different Cas9+gRNA combinations (from FIG. 4C). Data points show non-normalized read counts. Bars show mean±S.D. Data points are from three independent biological replicates. Data for TmcB1^{Bth/WT} is presented to the left of data for TMC1^{WT/WT} for each condition.

FIG. 13A illustrates targeted deep sequencing and CRISPResso analysis. FIG. 13A is a schematic illustrating PCR and sequencing of the Tmc1 gene. FIG. 13B is a schematic illustrating global indel analysis by CRISPREsso, wherein reads are not segregated. FIG. 13C provides a strategy to segregate Tmc1^{Bth} reads and Tmc1^{WT} reads. The region for splitting partly overlaps with indels, thus some reads cannot be assigned as mutant or WT. FIG. 13D is a schematic illustrating allele specific indel analysis by CRISPResso.

### DETAILED DESCRIPTION OF THE INVENTION

As described below, the present invention features a Cas9 variant capable of selectively targeting a mutant allele without disrupting the wildtype allele, and methods of using such variants for gene editing. In particular embodiments, the invention provides for the editing of dominant mutations associated with single nucleotide substitutions. The invention further provides methods and compositions for treating a disease or condition or symptoms thereof associated with a dominant mutation.

The invention is based, at least in part, on the discovery that a Cas9 variant (SaCas9-KKH) recognizes a non-canonical PAM sequence present in a TMC1 allele that carries a dominant mutation associated with progressive deafness and generates double-strand breaks in only the TMC1 alleles that carry the dominant allele. Importantly, SaCas9-KKH does not generate double strand breaks in the wild type allele lacking the PAM sequence. In an effort to identify Cas9 variants having the desired properties, 14 Cas9/gRNA combinations were screened for specific and efficient disruption of a nucleotide substitution that causes the dominant progressive hearing loss, DFNA36. As a model for DFNA36, Beethoven mice were used. Beethoven mice harbor a point mutation in Tmc1, a gene required for hearing that encodes a pore-forming subunit of mechanosensory transduction channels in inner ear hair cells. A PAM variant of Staphylococcus aureus Cas9 (SaCas9-KKH) was identified that selectively and efficiently disrupted the mutant allele, but not the wild-type Tmc1/TMC1 allele, in Beethoven mice and in a DFNA36 human cell line. AAV-mediated SaCas9-KKH delivery prevented deafness in Beethoven mice up to one year post transduction. Analysis of current ClinVar entries revealed that ˜21% of dominant human mutations could be targeted using a similar approach.

### Cas9

Cas9 is a nuclease, an enzyme specialized for cutting DNA, with two active cutting sites, one for each strand of the double helix. Jinek et al. (2012) combined tracrRNA and spacer RNA into a “single-guide RNA” molecule that, mixed with Cas9, could find and cut the correct DNA targets. It has been proposed that such synthetic guide RNAs might be able to be used for gene editing (Jinek et al., Science. 2012 Aug 17;337(6096):816-21).

Cas9 proteins are known in the art, such as Streptococcus pyogenes Cas9 (SpCas9), Staphylococcus aureus Cas9 (SaCas9), and Francisella novicida Cas9 (FnCas9). In general, Cas9 proteins preferentially interrogate and act upon DNA sequences containing a protospacer adjacent motif (PAM) sequence, and different Cas9 proteins have affinities for different PAMs. The canonical PAM sequence is 5′-NGG-3′, which is recognized by multiple Cas9 proteins, where N can be any nucleotide. For example, SpCas9 and FnCas9 recognize the canonical NGG PAM sequence. Streptococcus thermophilus Cas9 recognizes a 5′-NGA-3′ PAM sequence, and SaCas9 recognizes a 5′-NNGRR(N)-3′ PAM sequence. Additionally, Cas9 proteins can be modified to recognize PAM sequences that are distinct from the PAM sequences recognized by the unmodified Cas9 protein. For example, SaCas9-KKH recognizes a 5′-RRT-3′, where R denotes an adenosine or guanine nucleotide. The Cas9 nuclease used in the presently described methods will recognize a PAM sequence that is present only in the allele to be inactivated (i.e., the allele carrying a deleterious mutation). Thus, the nuclease activity of the Cas9 will act only upon the allele to be inactivated.

gRNA

A Cas9 protein, having an affinity for a particular PAM sequence can be directed to a particular locus in a genome by a guide RNA. In some embodiments, the guide RNA is a single guide RNA, which comprises a tracrRNA and a spacer RNA. The short spacer RNA, comprising a nucleic acid sequence that specifically binds to the target genomic locus, directs the Cas9 protein to the target, which is then cleaved by the Cas9 protein's nuclease activity. In some embodiments, synthetic gRNAs are about 18, 19, 20, 21, 22, 25, 30, 40, 50, 60, 70, 80, 90, 100, over 100 bp and comprise a nucleic acid sequence complementary to protospacer nucleotides near the PAM sequence

In some embodiments, the guide RNA will bind a nucleic acid sequence comprising a PAM sequence that is present in an allele carrying a mutation, but is not present in an allele that does not carry the mutation. In some embodiments, the guide RNA binds a nucleic acid sequence that is in close proximity to a PAM sequence that is present only in an allele to be inactivated (i.e., an allele carrying a deleterious mutation). The PAM sequence may be 1, 2, 3, 4, 5, 6, 7, 8, 9, or 10 nucleotides upstream or downstream of the sequence to which with guide RNA binds.

The following US patents and patent publications are incorporated herein by reference in their entireties: U.S. Pat. No. 8,697,359, 20140170753, 20140179006, 20140179770, 20140186843, 20140186958, 20140189896, 20140227787, 20140242664, 20140248702, 20140256046, 20140273230, 20140273233, 20140273234, 20140295556, 20140295557, 20140310830, 20140356956, 20140356959, 20140357530, 20150020223, 20150031132, 20150031133, 20150031134, 20150044191, 20150044192, 20150045546, 20150050699, 20150056705, 20150071898, 20150071899, 20150071903, 20150079681, 20150159172, 20150165054, 20150166980, and 20150184139.

### Polynucleotide Delivery

Therapeutic success in these approaches relies significantly on the safe and efficient delivery of exogenous gene constructs to the relevant therapeutic cell targets in the organ of Corti in the cochlea. The organ of Corti includes two classes of sensory hair cells: inner hair cells, which convert mechanical information carried by sound into electrical signals transmitted to neuronal structures and outer hair cells which serve to amplify and tune the cochlear response, a process required for complex hearing function.

Methods of delivering nucleic acids to cells generally are known in the art, and methods of delivering viruses (which also can be referred to as viral particles) containing a transgene to inner ear cells in vivo are described herein. As described herein, about 10⁸ to about 10¹² viral particles can be administered to a subject, and the virus can be suspended within a suitable volume (e.g., 10 μL, 50 μL, 100 μL, 500 μL, or 1000 μL) of, for example, artificial perilymph solution.

A virus containing a promoter (e.g., an Espin promoter, a PCDH15 promoter, a PTPRQ promoter, a Myo6 promoter, a KCNQ4 promoter, a Myo7a promoter, a synapsin promoter, a GFAP promoter, a CMV promoter, a CAG promoter, a CBH promoter, a CBA promoter, a U6 promoter, and a TMHS (LHFPL5) promoter) and a polynucleotide encoding a Cas9 protein (e.g., SaCas9-KKH), and in some embodiments, a guide RNA, as described herein can be delivered to inner ear cells (e.g., cells in the cochlea) using any number of means. For example, a therapeutically effective amount of a composition including virus particles containing one or more different types of transgenes as described herein can be injected through the round window or the oval window, or the utricle, typically in a relatively simple (e.g., outpatient) procedure. In some embodiments, a composition comprising a therapeutically effective number of virus particles containing a transgene (e.g., a polynucleotide encoding a transgene and a gRNA), or containing one or more sets of different virus particles, wherein each particle in a set can contain the same type of transgene, but wherein each set of particles contains a different type of transgene than in the other sets, as described herein can be delivered to the appropriate position within the ear during surgery (e.g., a cochleostomy or a canalostomy).

In addition, delivery vehicles (e.g., polymers) are available that facilitate the transfer of agents across the tympanic membrane and/or through the round window or utricle, and any such delivery vehicles can be used to deliver the viruses described herein. See, for example, Arnold et al., 2005, Audiol. Neurootol., 10:53-63.

The compositions and methods described herein enable the highly efficient delivery of nucleic acids to inner ear cells, e.g., cochlear cells. For example, a polynucleotide encoding a Cas9 protein, variant (e.g., SaCas9-KKH), or a fragment thereof, can be cloned into a retroviral vector and expression can be driven from its endogenous promoter, from the retroviral long terminal repeat, or from a promoter specific for a target cell type of interest. Other viral vectors that can be used include, for example, a vaccinia virus, a bovine papilloma virus, or a herpes virus, such as Epstein-Barr Virus. Retroviral vectors are particularly well developed and have been used in clinical settings. In some embodiments, a viral vector is used to administer a Cas9 polynucleotide systemically. In some embodiments, a viral vector is used to administer a Cas9 polynucleotide to a particular region of the body.

For example, the compositions and methods described herein enable the delivery to, and expression of, a KKH-Cas9 polynucleotide in at least 65% (e.g., at least 65, 70, 75, 80, 85, 90, 91, 92, 93, 94, 95, 96, 97, 98, or 99%) of inner and/or outer hair cells or delivery to, and expression in, at least 80% (e.g., at least 85, 90, 91, 92, 93, 94, 95, 96, 97, 98, or 99) of outer hair cells.

As demonstrated herein, expression of a KKH-Cas9 polynucleotide delivered using an AAV-vector can result in improved structure and function of inner and outer hair cells such that hearing is restored for an extended period of time (e.g., months, years, decades, a life time).

As described herein, an adeno-associated virus (AAV) are particularly efficient at delivering nucleic acids (e.g., polynucleotides encoding a Cas9 polypeptide and, in some embodiments, a gRNA) to inner ear cells. The Anc80 vector is an example of an Inner Ear Hair Cell Targeting AAV that advantageously transduced greater than about 60%, 70%, 80%, 90%, 95%, or even 100% of inner or outer hair cells. One particular ancestral capsid protein that falls within the class of Anc80 ancestral capsid protein is Anc80-0065 (SEQ ID NO:2) described in International Application No. PCT/US2018/017104, which is incorporated herein by reference in its entirety. However, WO 2015/054653, which is also incorporated herein by reference in its entirety, describes a number of additional ancestral capsid proteins that fall within the class of Anc80 ancestral capsid proteins.

In particular embodiments, the adeno-associated virus (AAV) contains an ancestral AAV capsid protein that has a natural or engineered tropism for hair cells. In some embodiments, the virus is an Inner Ear Hair Cell Targeting AAV, which delivers a transgene (e.g., a polynucleotide encoding a Cas9 polypeptide and, in some embodiments, a gRNA) to the inner ear in a subject. In some embodiments, the virus is an AAV that comprises purified capsid polypeptides. In some embodiments, the virus is artificial. In some embodiments, the virus is an AAV that has lower seroprevalence than AAV2. In some embodiments, the virus is an exome-associated AAV. In some embodiments, the virus is an exome-associated AAV1. In some embodiments, the virus comprises a capsid protein with at least 95% amino acid sequence identity or homology to Anc80 capsid proteins.

Expression of a Cas9 polynucleotide may be directed by a heterologous promoter (e.g., CMV promoter, Espin promoter, a PCDH15 promoter, a PTPRQ promoter and a TMHS (LHFPL5) promoter). As used herein, a “heterologous promoter” refers to a promoter that does not naturally direct expression of that sequence (i.e., is not found with that sequence in nature).

Methods for packaging a transgene into a virus that contains an Anc80 capsid protein are known in the art, and utilize conventional molecular biology and recombinant nucleic acid techniques. In one embodiment, a construct that includes a nucleic acid sequence encoding an Anc80 capsid protein and a construct carrying the polynucleotide encoding a Cas9 (and in some cases a Cas9 and a gRNA) flanked by suitable Inverted Terminal Repeats (ITRs) are provided, which allows for the transgene to be packaged within the Anc80 capsid protein.

The Cas9 polynucleotide (and in some embodiments, a Cas9 and a gRNA) can be packaged into an AAV containing an Anc80 capsid protein using, for example, a packaging host cell. The components of a virus particle (e.g., rep sequences, cap sequences, inverted terminal repeat (ITR) sequences) can be introduced, transiently or stably, into a packaging host cell using one or more constructs as described herein.

In some embodiments, a AAVs containing a AAV9-php.b vector is used to efficiently target inner ear cells. AAV9-php.b is described in International Application No. PCT/US2019/020794, the contents of which are incorporated herein by reference in their entirety. AAV-PHP.B encodes the 7-mer sequence TLAVPFK and efficiently delivers transgenes to the cochlea, where it showed remarkably specific and robust expression in the inner and outer hair cells. An AAV-PHP.B vector can comprise, but is not limited to, any of the promoters described herein.

Non-viral approaches can also be employed for the introduction of therapeutic to a cell of a patient requiring inactivation of an allele carrying a mutation associated with a disease or condition or symptom thereof. For example, a nucleic acid molecule can be introduced into a cell by administering the nucleic acid in the presence of lipofection.

Gene transfer can also be achieved using non-viral means involving transfection in vitro. Such methods include the use of calcium phosphate, DEAE dextran, electroporation, and protoplast fusion. Liposomes can also be potentially beneficial for delivery of DNA into a cell. Transplantation of normal genes into the affected tissues of a patient can also be accomplished by transferring a normal nucleic acid into a cultivatable cell type ex vivo (e.g., an autologous or heterologous primary cell or progeny thereof), after which the cell (or its descendants) are injected into a targeted tissue.

cDNA expression for use in polynucleotide therapy methods can be directed from any suitable promoter (e.g., the human cytomegalovirus (CMV), simian virus 40 (SV40), or metallothionein promoters), and regulated by any appropriate mammalian regulatory element. For example, if desired, enhancers known to preferentially direct gene expression in specific cell types can be used to direct the expression of a nucleic acid. The enhancers used can include, without limitation, those that are characterized as tissue- or cell-specific enhancers. Alternatively, if a genomic clone is used as a therapeutic construct, regulation can be mediated by the cognate regulatory sequences or, if desired, by regulatory sequences derived from a heterologous source, including any of the promoters or regulatory elements described above.

Another therapeutic approach included in the invention involves administration of a recombinant therapeutic, such as a recombinant a Cas9 protein, variant, or fragment thereof, either directly to the site of a potential or actual disease-affected tissue or systemically (for example, by any conventional recombinant protein administration technique). The dosage of the administered protein depends on a number of factors, including the size and health of the individual patient. For any particular subject, the specific dosage regimes should be adjusted over time according to the individual need and the professional judgment of the person administering or supervising the administration of the compositions.

### Inactivating Alleles

Compositions and methods are provided herein for altering a cell to inactivate a mutant allele associated with a disease or condition using a CRISPR-Cas system. In some embodiments, a Cas9 (e.g., SaCas9-KKH), in combination with a single guide RNA is used to target an allele comprising a mutation. To selectively inactivate the allele carrying the mutation, while not inactivating the wild type or other non-deleterious forms of the allele, a Cas protein is used that recognizes a PAM sequence present in the mutant allele but not in the wild type (or other non-mutant form) allele. Upon target recognition, the Cas protein (e.g., Cas9) induces at least one double strand break in the target mutant allele. Repair of the double-strand break by non-homologous end joining (NHEJ) increases the probability of an indel at the double-strand break site. In some embodiments, an indel at the double-strand break site generates a premature stop codon in the mutant allele that inactivates the mutant allele. In some embodiments, the indel can be in a regulatory region of the allele that results in inhibited expression of the allele. In some embodiments, the indel generates a protein product that is lacks a deleterious nature (i.e., the edited allele does not interfere with the expression and function of the wildtype (or non-mutant form) allele.

### Compositions and Methods of Treatment

The present invention provides methods of treating disease and/or disorders or symptoms thereof which comprise administering a therapeutically effective amount of a pharmaceutical composition comprising a Cas9 nuclease and a guide RNA that specifically binds a nucleic acid sequence in the genome comprising a mutation and a PAM sequence recognized by the Cas9 to a subject (e.g., a mammal such as a human), wherein the PAM sequence is not present in the allele that does not carry the mutation. Thus, one embodiment is a method of treating a subject suffering from or susceptible to a disease or disorder or symptom thereof associated with a mutation. The method includes administering to the mammal a therapeutic amount of an amount of a compound herein sufficient to treat the disease or disorder or symptom. In some embodiments, the mutation is a dominant mutation.

The therapeutic methods of the invention (which include prophylactic treatment), in general, comprise administration of a therapeutically effective amount of the compounds herein, such as a compound of the formulae herein to a subject (e.g., animal, human) in need thereof, including a mammal, particularly a human. Such treatment will be suitably administered to subjects, particularly humans, suffering from, having, susceptible to, or at risk for a disease, disorder, or symptom thereof. Determination of those subjects “at risk” can be made by any objective or subjective determination by a diagnostic test or opinion of a subject or health care provider (e.g., genetic test, enzyme or protein marker, Marker (as defined herein), family history, and the like).

Compositions are contemplated herein for the treatment of diseases or conditions associated with a mutation. For therapeutic purposes, compositions comprising a Cas9 polypeptide, or a polynucleotide encoding a Cas9 polypeptide and a guide RNA that specifically binds to a nucleic acid sequence in a genome that comprises a mutation that causes or contributes to a disease or condition (e.g., dominant progressive hearing loss) as described herein may be administered directly to a region of the body (e.g., cochlea) that is affected by the disease or condition. In some embodiments, the compositions are formulated in a pharmaceutically-acceptable buffer such as physiological saline. Non-limiting methods of administration include injecting into the cochlear duct or the perilymph-filled spaces surrounding the cochlear duct (e.g., scala tympani and scala vestibuli). Injecting into the cochlear duct, which is filled with high potassium endolymph fluid, could provide direct access to hair cells. However, alterations to this delicate fluid environment may disrupt the endocochlear potential, heightening the risk for injection-related toxicity. The perilymph-filled spaces surrounding the cochlear duct, scala tympani and scala vestibuli, can be accessed from the middle ear, either through the oval or round window membrane. The round window membrane, which is the only non-bony opening into the inner ear, is relatively easily accessible in many animal models and administration of viral vector using this route is well tolerated. In humans, cochlear implant placement routinely relies on surgical electrode insertion through the round window membrane.

Treatment of human patients or non-human animals are carried out using a therapeutically effective amount of a combination therapeutic in a physiologically-acceptable carrier. The phrase “pharmaceutically acceptable” refers to those compounds of the invention, compositions containing such compounds, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically-acceptable excipient” includes pharmaceutically-acceptable material, composition or vehicle, such as a liquid or solid filler, diluent, carrier, solvent or encapsulating material, involved in carrying or transporting the subject compound from one organ, or portion of the body, to another organ, or portion of the body. Each carrier must be “acceptable” in the sense of being compatible with the other ingredients of the formulation and not injurious to the patient. Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

The compositions may conveniently be presented in unit dosage form and may be prepared by any methods well known in the art. The amount of active ingredient which can be combined with a carrier material to produce a single dosage form will vary depending upon the host being treated, the particular mode of administration. The amount of active ingredient which can be combined with a carrier material to produce a single dosage form will generally be that amount of the compound which produces a therapeutic effect. Generally, out of one hundred per cent, this amount will range from about 1 per cent to about ninety-nine percent of active ingredient, preferably from about 5 per cent to about 70 per cent, most preferably from about 10 per cent to about 30 per cent.

Additional suitable carriers and their formulations are described, for example, in the most recent edition of Remington's Pharmaceutical Sciences by E. W. Martin. The amount of the therapeutic agent to be administered varies depending upon the manner and mode of administration, the age and disease status (e.g., the extent of hearing loss present prior to treatment).

Compositions are administered at a dosage that controls the clinical or physiological symptoms of the disease or condition, as may in some cases be determined by a diagnostic method known to one skilled in the art.

Therapeutic compounds and therapeutic combinations are administered in an effective amount. For example, about 10⁸ to about 10¹² viral particles can be administered to a subject, and the virus can be suspended within a suitable volume (e.g., 10 μL, 50 μL, 100 μL, 500 μL, or 1000 μL) of, for example, artificial perilymph solution.

### Methods of Treating Dominant Progressive Hearing Loss

Compositions and methods for treating dominant progressive hearing loss (e.g., Deafness, Autosomal Dominant 36, or dominant progressive deafness 36, (DFNA36)) are provided.

DFNA36 is associated with dominant mutations (acquired or inherited) in the TMC1 gene of affected individuals. To inactivate the dominant mutation in a heterozygous subject, a SaCas9-KKH protein along with a guide RNA that recognizes the genomic locus containing the TMC1 dominant mutant, is administered to a subject as described above. The SaCas9=KKH protein that recognizes the RRT PAM sequence present in the TMC1 allele carrying the dominant mutation. Mutations within the TMC1 gene can cause Deafness, Autosomal Dominant 36 (DFNA36), a dominant progressive form of deafness. The SaCas9-KKH protein binds to a cleaves the TMC1 allele carrying the dominant mutation (but not the wild type allele), which promotes indel formation at the break site during non-homologous end joining. Resulting premature stop codons generate truncated, non-functional TMC1 proteins that are not dominant to the expressed wild type protein.

In some embodiments, the SaCas9-KKH nuclease is administered to a subject by directly injecting a vector (e.g., AAV or lentiviral vector) encoding the SaCas9-KKH protein and a guide RNA into the cochlea of the subject. In some embodiments, the vector only encodes the SaCas9-KKH protein and the guide RNA is administered in the injection as RNA For therapeutic purposes, compositions comprising a Cas9 polypeptide, or a polynucleotide encoding a Cas9 polypeptide and a guide RNA that specifically binds to a nucleic acid sequence in a genome that comprises a mutation that causes or contributes to dominant progressive hearing loss as described herein may be administered directly to a region of the body (e.g., cochlea) that is affected by the disease or condition. Non-limiting methods of administration include injecting into the cochlear duct or the perilymph-filled spaces surrounding the cochlear duct (e.g., scala tympani and scala vestibuli). Injecting into the cochlear duct, which is filled with high potassium endolymph fluid, could provide direct access to hair cells. However, alterations to this delicate fluid environment may disrupt the endocochlear potential, heightening the risk for injection-related toxicity. The perilymph-filled spaces surrounding the cochlear duct, scala tympani and scala vestibuli, can be accessed from the middle ear, either through the oval or round window membrane. The round window membrane, which is the only non-bony opening into the inner ear, is relatively easily accessible in many animal models and administration of viral vector using this route is well tolerated. In humans, cochlear implant placement routinely relies on surgical electrode insertion through the round window membrane.

In some embodiments, inactivating the mutant allele that causes DFNA36 while expressing the wildtype allele can restore auditory function in a subject. In some embodiments, the auditory function restored to a subject is about 10%, about 20%, about 30%, about 40%, about 50%, about 60%, about 70%, about 80%, about 90%, or even about 100%.

### EXAMPLES

### Example 1: Screening Cas9 and gRNA Combinations In Vitro

In order to develop allele specific genome editing strategies for dominant hearing loss, the Beethoven (Bth) mouse was used. The Bth mouse provides an excellent model for DFNA36 hearing loss in humans. The Bth mutation results in an amino acid substition (p.M412K, c.T1253A) in Tmc1. The mutation causes hair cell degeneration and progressive hearing loss in mice. In humans, the p.M418K substitution is identical to the Bth mutation in the orthologous position and causes DFNA36, dominant progressive hearing loss.

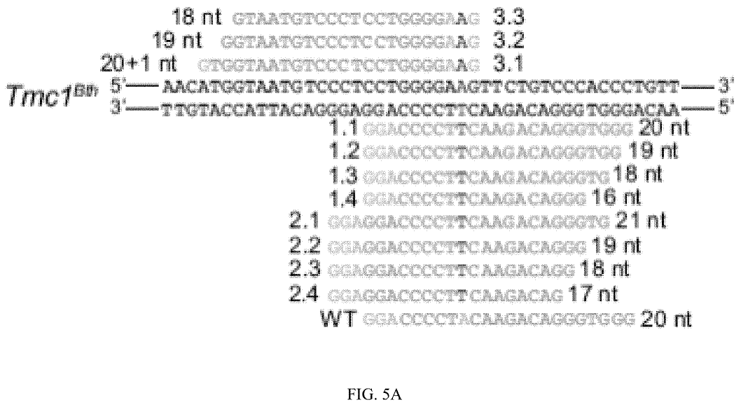

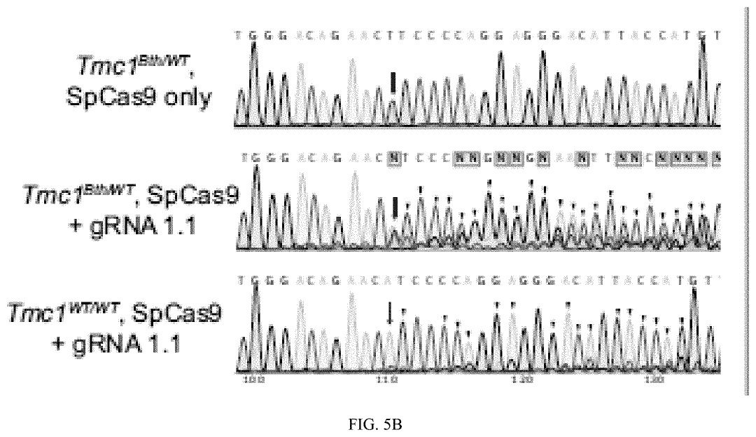

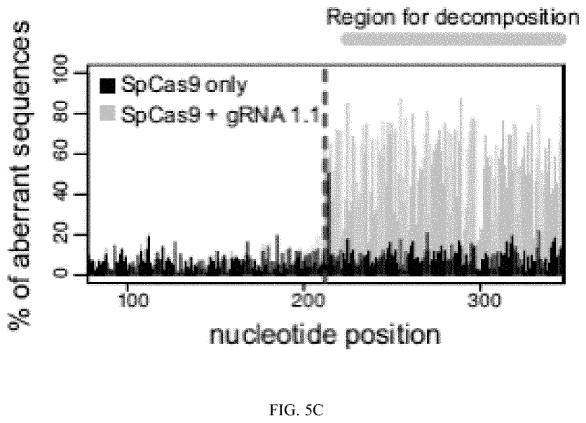

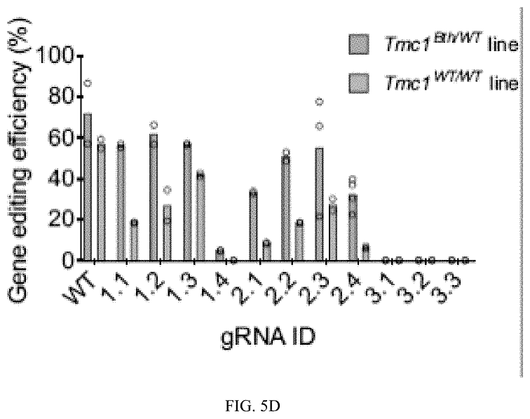

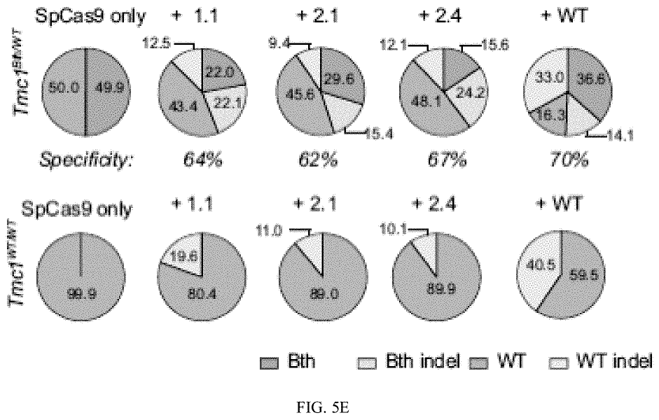

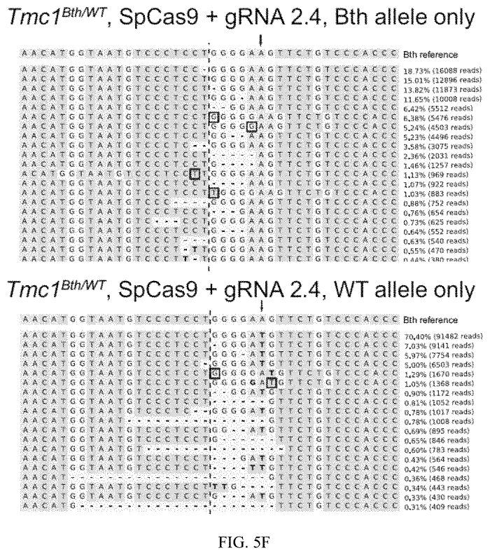

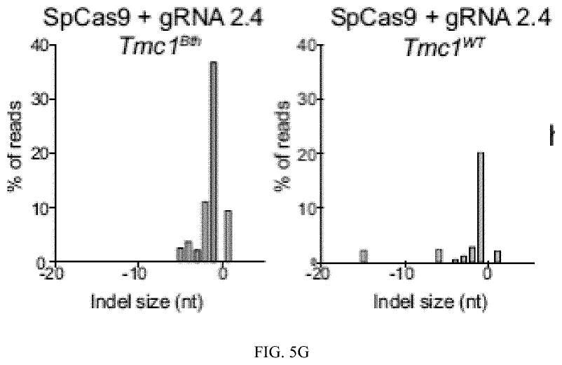

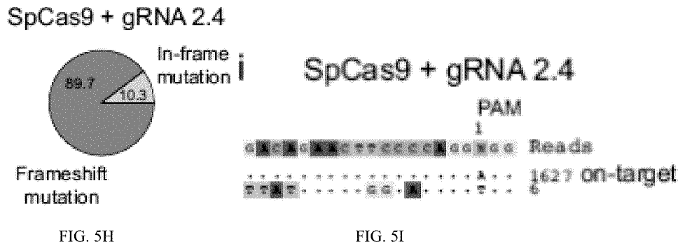

To selectively disrupt the Bth allele in fibroblasts from Tmc1^{Bth/WT} mice (Tmc1^{WT/WT} cells were used as controls), various Cas9 and gRNA combinations were screened in vitro. Plasmids encoding SpCas9-2A-GFP, along with the different gRNAs, were transfected into fibroblasts in duplicate. Four days after transfection, GFP-positive cells were sorted by fluorescence assisted cell sorting (FACS). SpCas9 in combination with 12 different gRNAs were tested, including full-length and truncated forms targeting either Tmc1^{Bth} or Tmc1^{WT} (FIG. 5A). Two independent sequencing reactions (forward and reverse) were performed. However, none of the various combinations, including the Cas9:gRNA1.1 combination previously reported to show transient improvement in auditory thresholds in Bth mice, had the necessary specificity, as indel formation was evident in both Bth and WT alleles (FIG. 5B). Sequencing data was further analyzed using Tracking of Indels by Decomposition (TIDE), and no indel formation was observed in the case of 3.1, 3.2 and 3.3 gRNAs. gRNA 1.4 showed minimal, but specific genome editing on the Tmc1Bth/WT cells. All the other gRNAs mediated efficient indel formation both in Tmc1Bth/WT or Tmc1WT/WT cells (FIGS. 5C and 5D).

Targeted deep sequencing was performed on control (SpCas9-2A-GFP only) cells, WT gRNA and the 3 most specific gRNAs (1.1, 2.1 and 2.4) in Tmc1^{Bth/WT} (top) Tmc1^{WT/WT} (bottom) cells. Indels were quantified after segregating Tmc1^{Bth} and Tmc1 ^{WT} reads by CRISPResso (only insertions and deletions were quantified, substitutions were ignored). None of the gRNAs are specific to the Tmc1^{Bth} allele, and mediate efficient indel formation on the Tmc1^{WT} allele as well. Sequencing was performed one time from pooled cells, transfected in triplicates. Specificity was defined as the indel percentage towards the targeted allele among total indels. The gRNA with the highest selectivity towards the Tmc1^{Bth} allele was gRNA 2.4 (FIG. 5E). The most abundant reads in the SpCas9+gRNA 2.4 treated cells were aligned to Bth allele. WT reads appear as having a substitution (a T to A change). Indel formation is evident in both Tmc1^{Bth} and Tmc1^{WT} reads (FIG. 5F). Referring to FIG. 5G, the most common indel event identified in the indel profiles from SpCas9+gRNA 2.4 transfected Tmc1^{Bth/WT} fibroblasts is a single base deletion. The percentages of indels causing in-frame and frame shift mutations in the coding sequence of Tmc1 after SpCas9+gRNA 2.4 transfection is shown in FIG. 5H. GUIDE-Seq analysis was performed on SpCas9+gRNA 2.4 transfected Tmc1^{Bth/WT} fibroblasts. Genomic DNA was isolated from 3 biological replicates for sequencing on one occasion. Only one off-target site was identified (FIG. 5I).

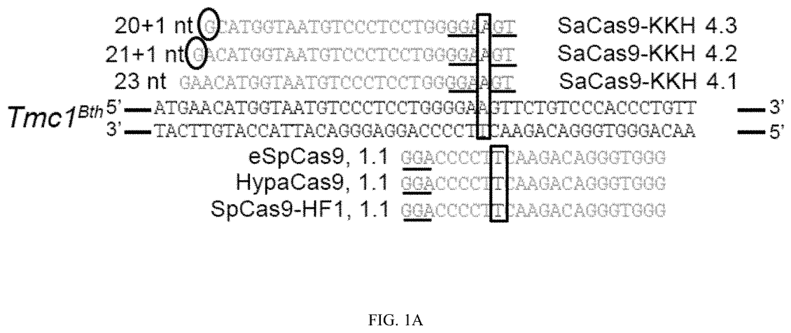

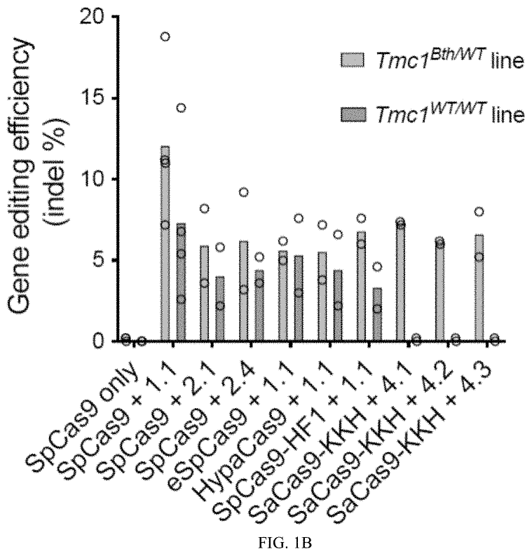

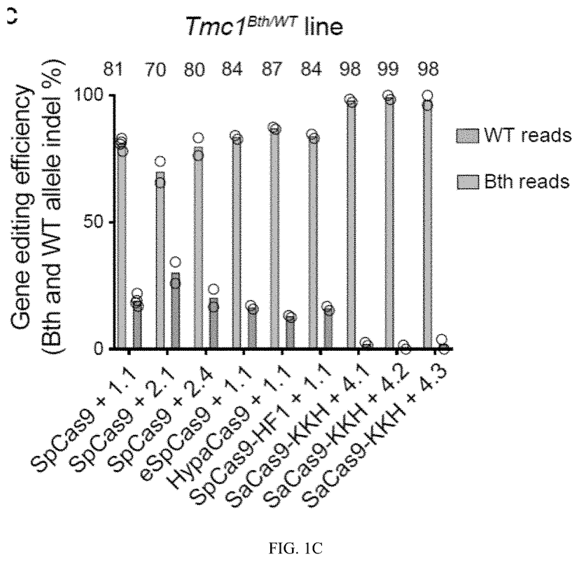

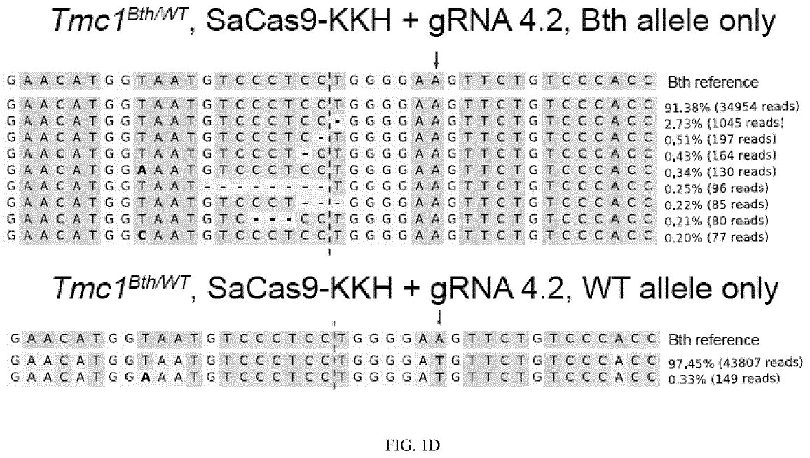

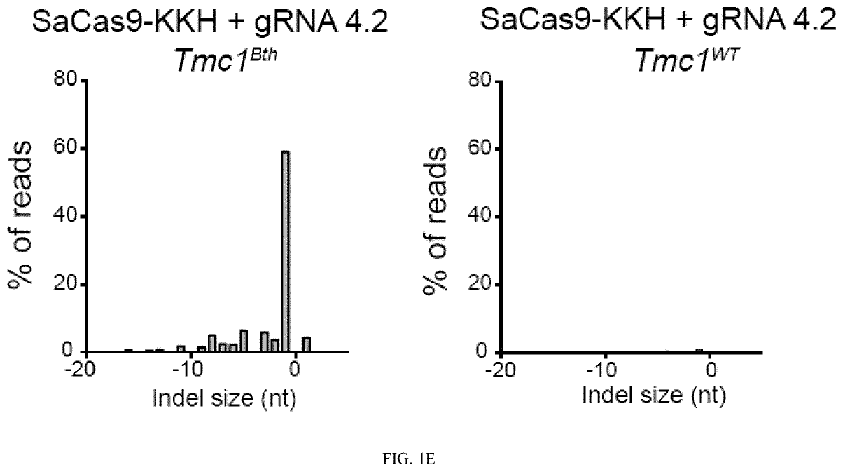

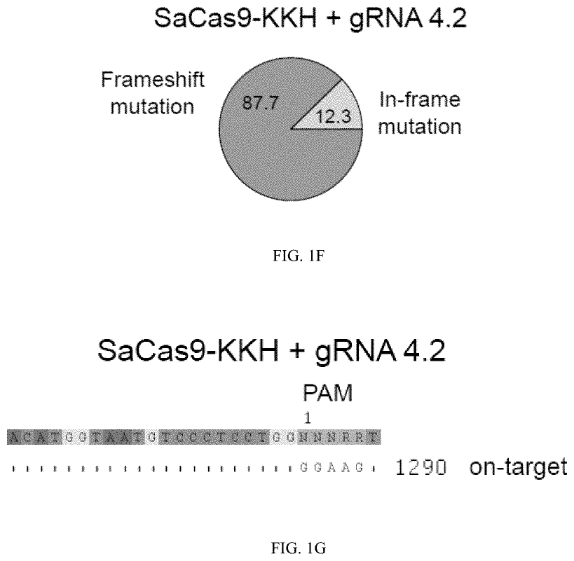

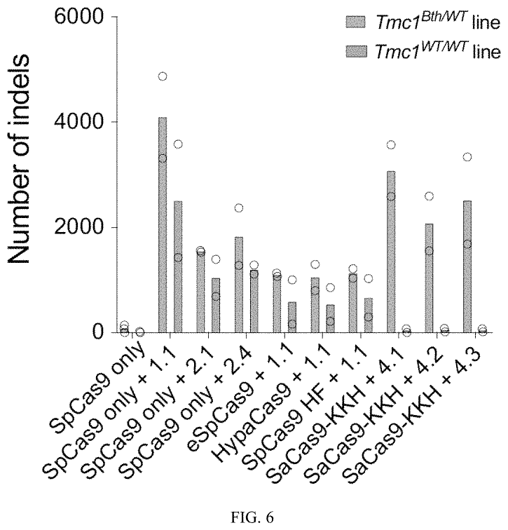

To improve allele selectivity, high-fidelity SpCas9 enzymes were also evaluated; however, none mediated selective targeting of the Bth allele (FIGS. 1A, 1B, and FIG. 6).

### Example 2: SaCas9-KKH Recognizes a PAM Site Selective for the TMC^{Bth} Allele

It was hypothesized that a Cas9 nuclease with a PAM site selective for the mutant sequence might show specific targeting of the Tmc1^{Bth} allele. The Bth mutation is a T to A change; thus, the GGAAGT sequence present in Tmc1^{Bth}, but not in Tmc1^{Bth} (GGATGT), may allow the PAM site of the SaCas9-KKH variant (NNNRRT) to distinguish the Tmc1^{Bth} from the Tmc1^{WT} allele. Full-length and truncated gRNAs were designed (FIG. 1A) and plasmids expressing each of these together with SaCas9-KKH were transfected into fibroblasts. SaCas9-KKH induced indels in Tmc1^{Bth/WT} , but not in Tmc1^{WT/WT} fibroblasts (FIG. 1B and FIG. 6). This method revealed high sensitivity, as the indel rates in CRISPR treated samples were 40-160-fold higher than the background indel rates observed in untreated samples.

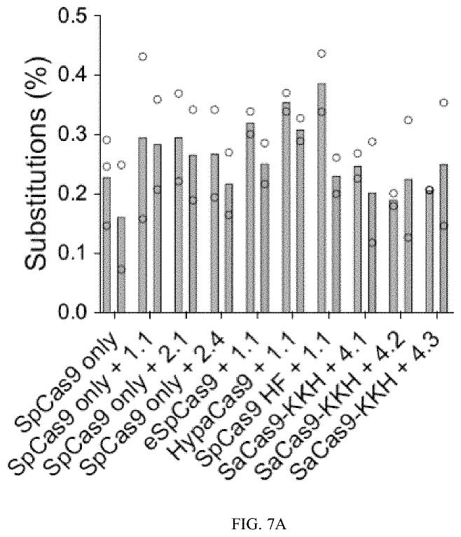

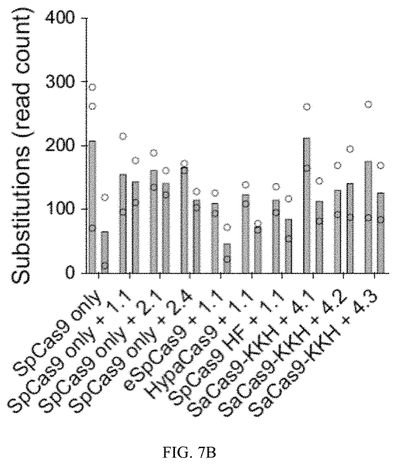

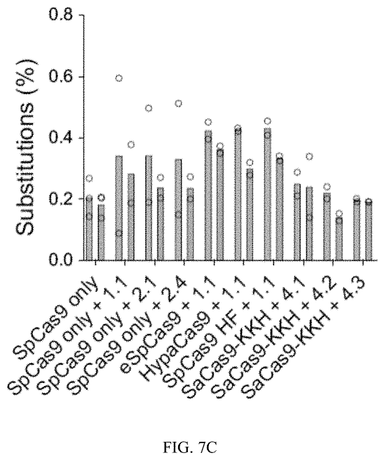

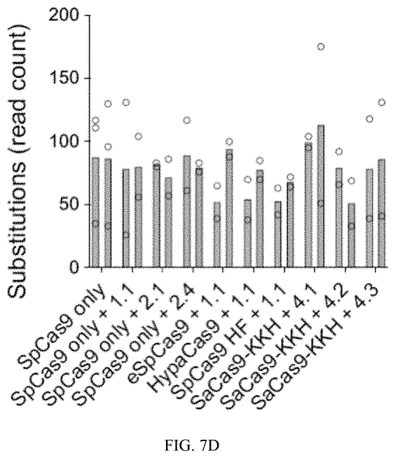

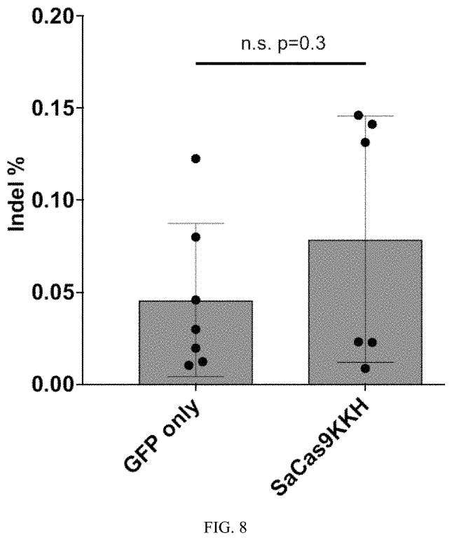

Allele-specific indel formation in Tmc1^{Bth/WT} cells was analyzed to avoid potential differences in transfection efficiency between Tmc1^{Bth/WT} and Tmc1^{WT/WT} fibroblasts. Tmc1^{Bth} and Tmc1 ^{WT}sequencing reads were segregated using a Python script and indel percentages were analyzed for each allele. (FIG. 1C). Allele-specific analysis revealed that 98-99% of all indels that occurred in Tmc1^{Bth/WT} cells were present just in the mutant allele. The indel profile revealed that the majority of CRISPR-induced variants were deletions, the most common being a single base deletion causing a frame-shift. (FIGS. 1D-1F). Single nucleotide changes were not frequent events (0.1-0.5% of reads) and there was no difference between Cas9-treated and control cells in the percentage or number of reads with single nucleotide substitutions (FIGS. 7A-7D). The few indels (less than 0.2%) in Tmc1^{WT/WT} fibroblasts were not significantly different from indel rates in no-gRNA control samples, which likely reflect PCR/sequencing error (FIG. 8). Importantly, SaCas9-KKH+gRNA 4.2 was specific for the Tmc1^{Bth} allele in transfected fibroblasts. Genomic DNA was pooled from three biological replicates for sequencing. Cleavage was not detected at any genome-wide off-target sites using GUIDE-Seq (FIG. 1G).

### Example 3: SaCas9-KKH-Mediated Indel Formation in Sensory Hair Cells

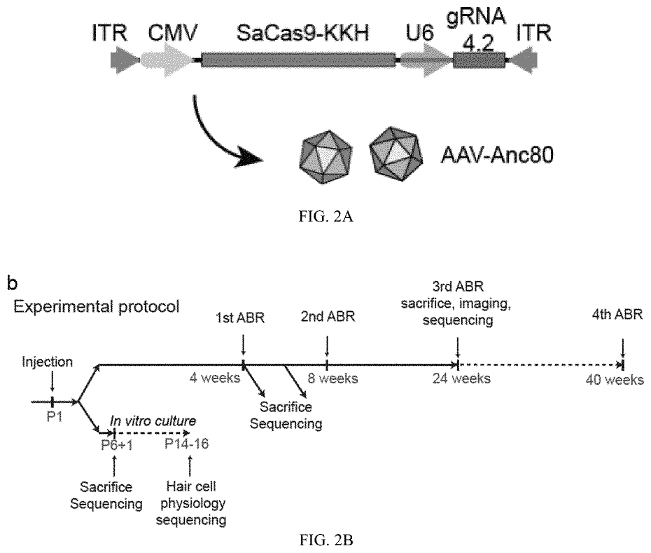

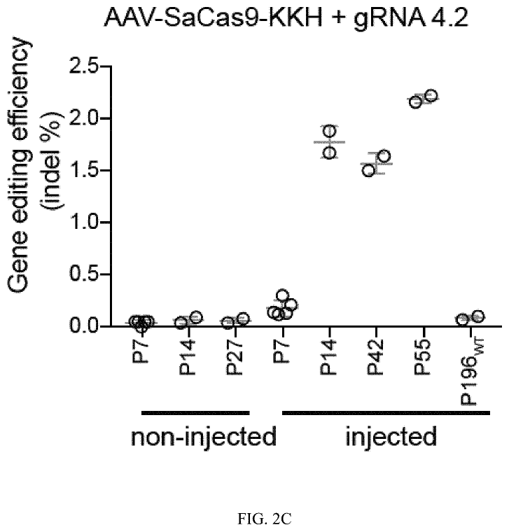

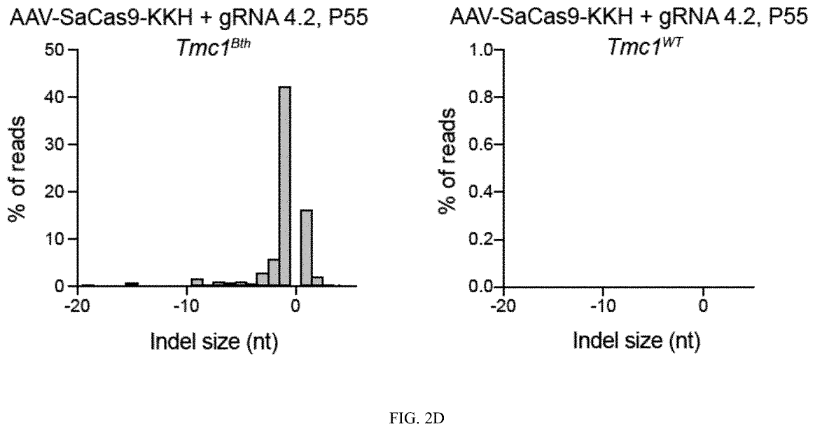

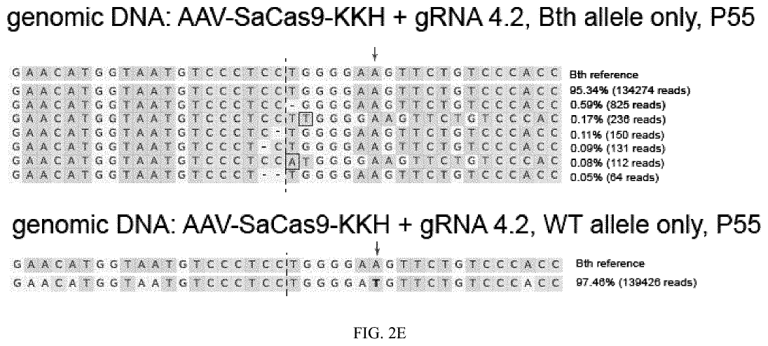

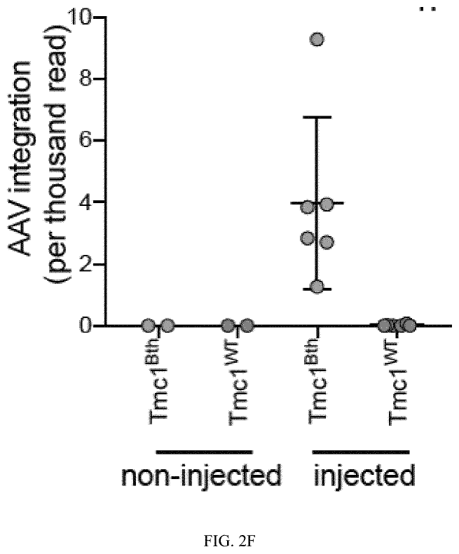

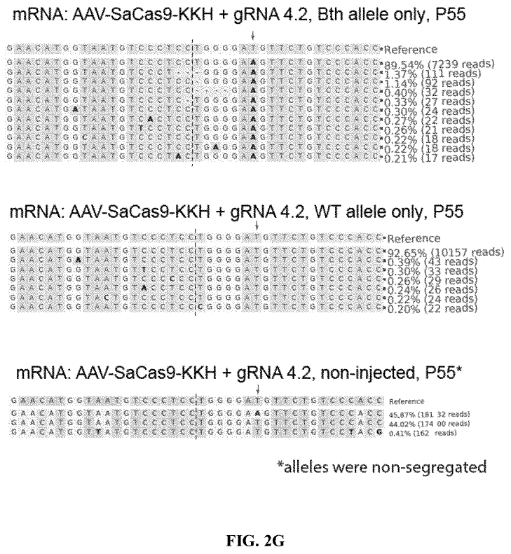

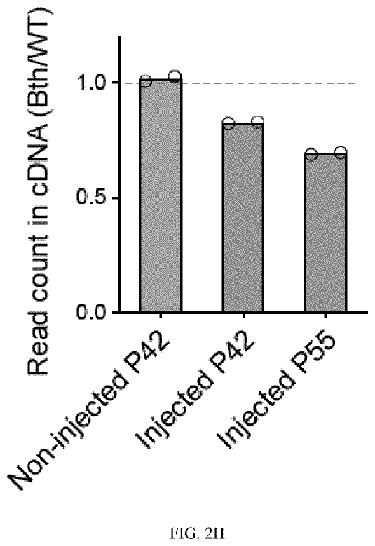

To assess the capability of SaCas9-KKH and gRNA 4.2 to introduce indels into the TMC1 gene, the Cas9 protein and guide RNA were packaged into Anc80L65 capsids (FIG. 2A) for delivery to sensory hair cells of the cochlea. 1 μl of virus was injected into the inner ears of P1 mice (FIG. 2B). Targeted deep sequencing from whole cochlear tissue at different ages post-injection and in non-injected animals (FIG. 2C) was performed. In whole cochlea, in which supporting cells vastly outnumber viral-targeted hair cells, 0.2%, 1.8%, 1.6% and 2.2% indel frequencies at 7, 14, 42 and 55 days after injection were observed, respectively. Indel formation in injected WT animals was not different from background even after 196 days (FIG. 2C). Indel formation was detected in the Tmc1^{Bth} allele but not in the Tmc1^{WT} allele in injected Tmc1^{Bth/WT} animals (FIGS. 2D, 2E). A more sensitive, independent analysis—the presence of AAV inverted terminal repeats in the cut site—was used to investigate allele selectivity of SaCas9-KKH. In non-injected Tmc1^{Bth/WT} animals, AAV reads within the Tmc1 gene were not detected (FIG. 2F) but AAV integration was evident in the Tmc1^{Bth} allele in all injected animals at P42 or P55. However, only three unique Tmc1^{WT} reads with AAV integration in 45 injected mice were observed, corresponding to a 0.0075% indel rate, suggesting little SaCas9-KKH activity on the WT allele. As a final test, gene editing at the mRNA level was analyzed. In contrast to non-injected Tmc1^{Bth/WT} animals, some indel formation was observed at the mRNA level in injected animals at P55 (0.83% in injected animals, FIG. 2G), but only in mutant alleles. A 24% decrease (FIG. 2H) in non-modified Bth mRNA relative to non-modified WT mRNA in injected animals was also observed.

### Example 4: SaCas9-KKH-Guide RNA Significantly Reduces Auditory Brainstem Responses

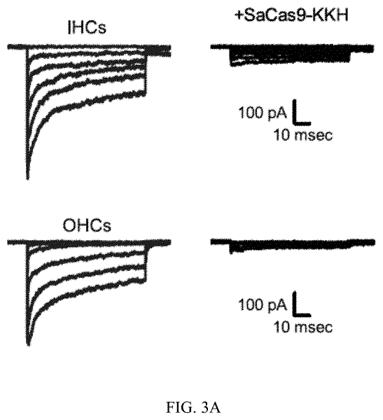

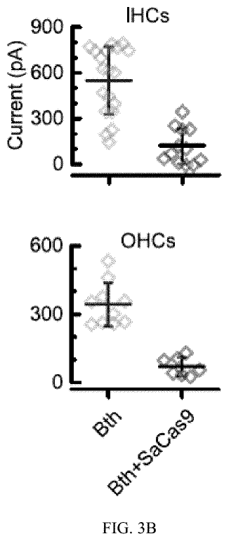

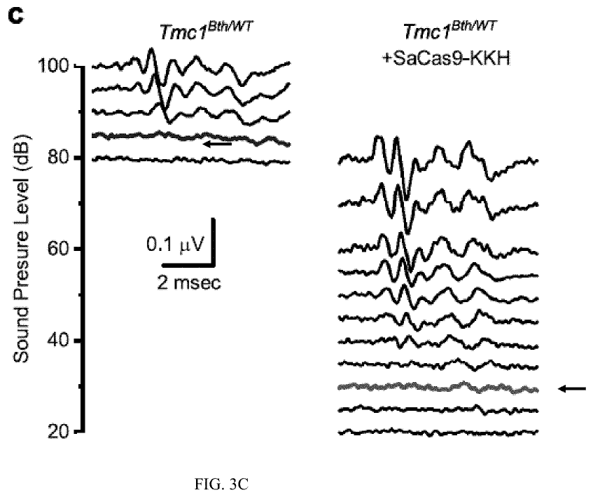

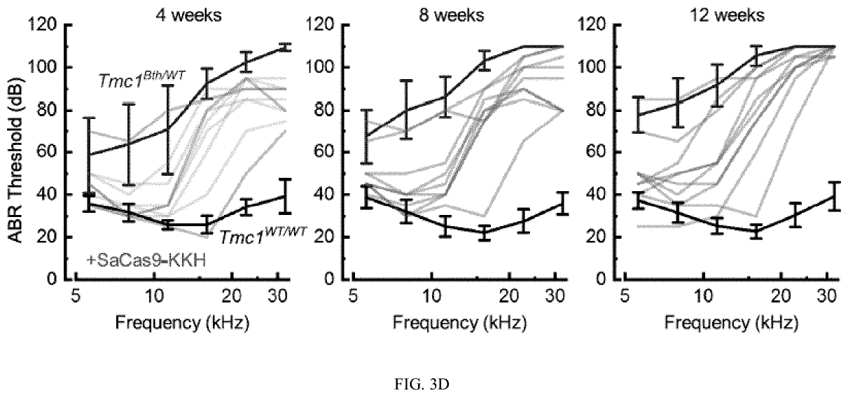

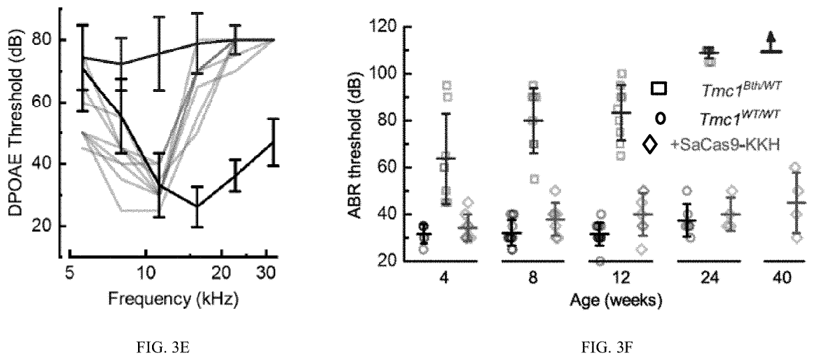

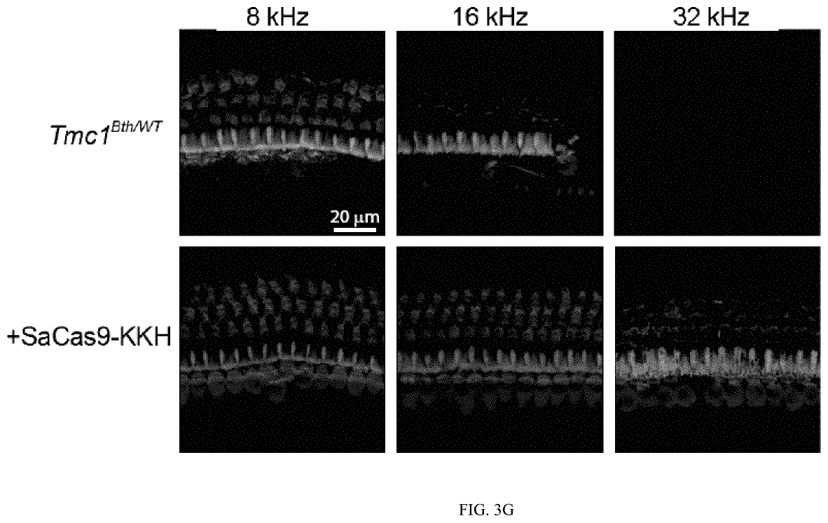

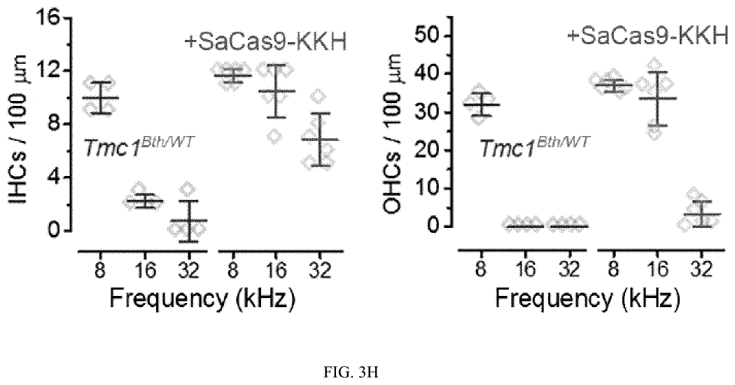

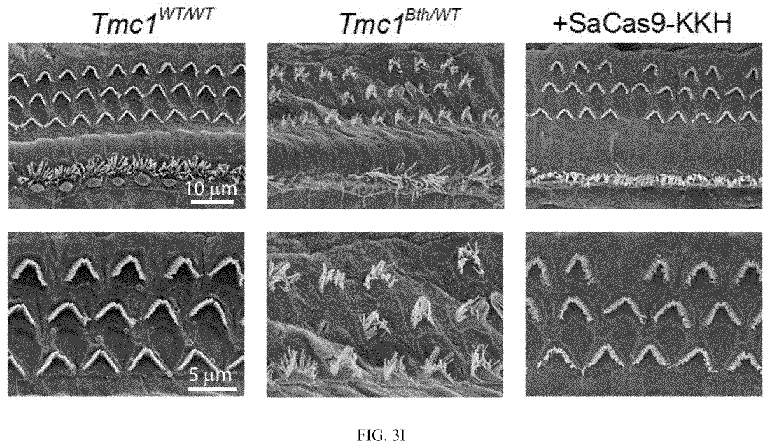

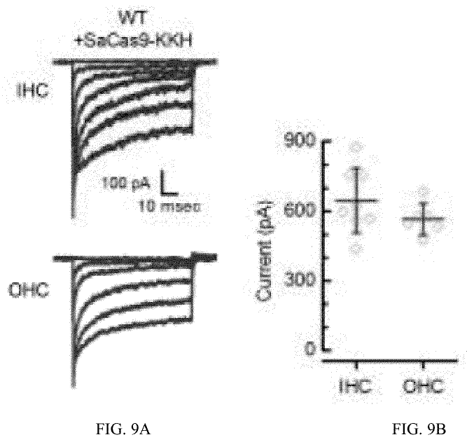

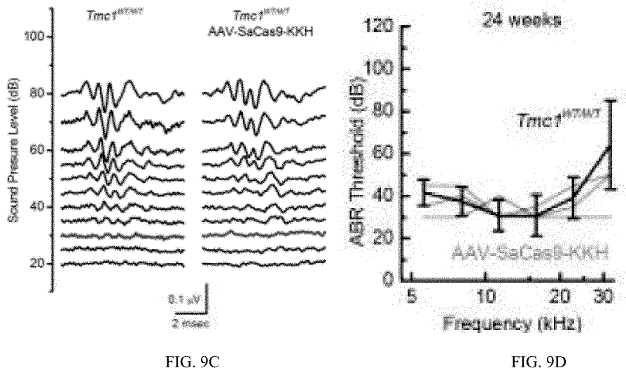

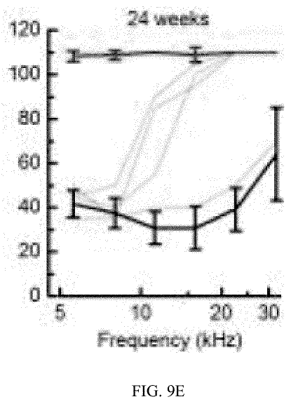

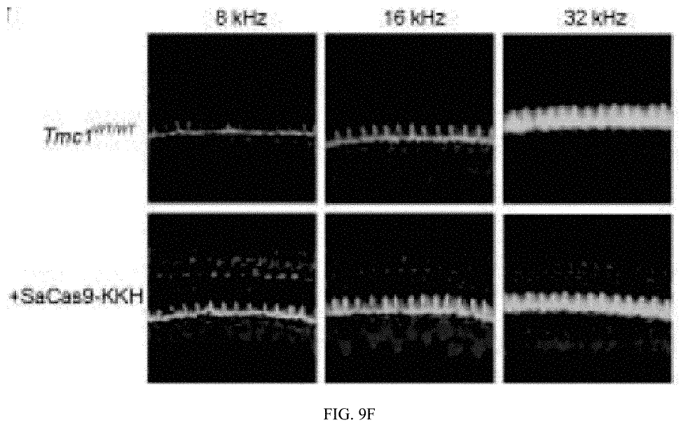

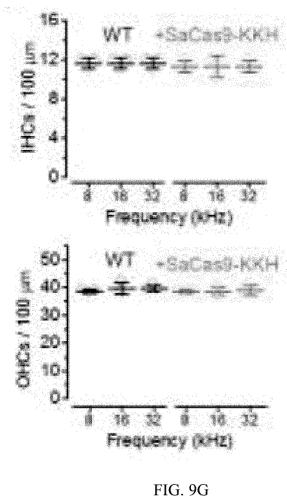

SaCas9-KKH-mediated disruption of the Bth allele was evaluated using hair cell mechanosensory transduction current. Although the Bth mutation eventually causes cell death, the mutation does not cause a loss of mechanosensitivity. Single-cell electrophysiology was performed on hair cells from either Tmc1^{WT/WT} or Tmc1^{Bth/Δ} mouse pups on a Tmc2^{Δ/Δ} background because Tmc2 contributes to mechanosensory currents and is expressed transiently at neonatal stages. After injection of AAV-SaCas9-KKH-gRNA-4.2 at P1, cochleas were dissected at P5-P7 and cultured 8-10 days, or the equivalent of P14-P16. Both inner and outer hair cells from injected Tmc1^{WT/WT} mice showed normal current amplitudes (FIGS. 9A, 9B) similar to WT amplitudes reported previously, which indicated no disruption of the Tmc1^{WT} allele. Tmc1^{Bth/Δ}; Tmc2^{Δ/Δ} hair cells from mice injected with AAV-CMV-SaCas9-KKH-U6-gRNA-4.2 showed a significant reduction in current amplitude in both inner and outer hair cells, relative to hair cells of uninjected Tmc1^{Bth/Δ}; Tmc2^{Δ/Δ} mice, in some cases almost completely abolishing the current (FIGS. 3A, 3B).

### Example 5: SaCas9-KKH-Guide RNA Improves Auditory Brain Responses

Auditory brainstem responses (ABR) and distortion product otoacoustic emissions (DPOAE) were evaluated in Bth mice using the allele-specific SaCas9-KKH nuclease (FIG. 2B). In 8-week old Tmc1^{Bth/WT} mice, ABR recordings revealed elevated hearing thresholds (90 dB at 8 kHz; FIG. 3C) compared to WT controls (30 dB, FIG. 9C), consistent with the progressive hearing loss in Bth mice. In Tmc1^{Bth/WT} animals injected with AAV-SaCas9-KKH-gRNA-4.2, improved thresholds were observed at eight weeks (45 dB median threshold; FIGS. 3C, 3D). At four weeks, the mouse with the greatest hearing preservation had ABR thresholds of 20-35 dB in the 5-16 kHz test range, indistinguishable from wild-type mice (FIG. 3D).

Since the Bth mutation causes progressive hearing loss, the time course of hearing sensitivity in Tmc1^{Bth/WT} and Tmc1^{WT/WT} animals was measured at 4, 8, 12 and 24 weeks after injection at frequencies from 5.6 to 32 kHz (FIGS. 3D, 3F and FIGS. 9D, 9E). At 4 weeks of age, uninjected Bth mice had low frequency hearing but high frequency hearing loss (FIG. 3D), similar to previous reports. At later time points, ABR thresholds were progressively elevated, and at 24 weeks of age, no ABR thresholds were detected in untreated Bth mice (FIG. 9E). In contrast, Bth mice injected with AAV-SaCas9-KKH-gRNA-4.2 showed normal or near-normal ABR thresholds at low frequencies (8 kHz, injected: 38±11 dB n=9; uninjected: 64±19 dB n=8) and improved, but not completely normalized, ABR thresholds at high frequencies (22kHz, injected: 84±15 dB n=9; uninjected: 103±5 dB n=8). In contrast to uninjected Bth mice, ABR thresholds in injected mice did not deteriorate over time (8 kHz, 8 weeks: 45±15 dB n=9; 12 weeks: 48±17 dB n=10; 24 weeks: 40±7 dB, n=4; FIG. 3F). At 24 weeks, injected mice exhibited normal or near-normal thresholds at 5-8 kHz, while untreated animals were profoundly deaf. One injected animal showed remarkable preservation of hearing even in high frequencies at 24 weeks of age (FIG. 9E).

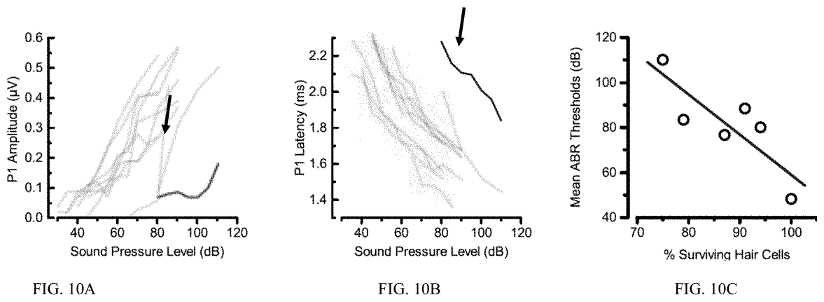

ABR peak 1 (P1) amplitudes were in the normal range for most of the injected Bth mice at 8 weeks, in contrast to non-injected animals, which showed small P1 amplitudes only at high sound intensities (FIG. 10A). Latencies of P1 waves of injected Tmc1^{Bth/WT} animals were also normalized by injection (FIG. 10B). To test outer hair cell function, DPOAE was measured at 4, 8, 12 and 24 weeks after injection. Like the ABRs, DPOAE thresholds in injected Bth mice revealed preservation of outer hair cell function at lower frequencies (5-11 kHz) at 12 weeks of age (FIG. 3E) and in the surviving animals, up to 24 weeks of age.

Whether AAV-SaCas9-KKH-gRNA-4.2 injection disrupts hearing in wild-type animals was also investigated. ABRs were performed and no ABR or DPOAE threshold shifts were observed, even 24 weeks post-injection (FIG. 9D, 8 kHz, injected: 38±8 dB n=3; uninjected: 38±6 dB n=6) confirming that AAV-SaCas9-KKH does not disrupt hearing function in Tmc1^{WT/WT} mice.

In one cohort of four Bth mice, ABR thresholds were measured at 40 weeks post-injection. Thresholds (8 kHz) between 4 and 40 weeks were tracked and data from two mice with failed injections was excluded, based on histological examination (below). Thresholds were stable over time and only slightly elevated relative to WT mice (FIG. 3F). Two of six mice that survived to one year of age had stable ABR thresholds of 35 and 40 dB at 8 kHz.

Next, hair cell survival was evaluated in injected and non-injected Tmc1^{Bth/WT} and Tmc1^{WT/WT} animals. Following ABR and DPOAE evaluations, mice were sacrificed at 24 weeks of age. Surviving hair cells were identified with an antibody for MYO7A and phalloidin staining for actin. Inner and outer hair cells were present in uninjected Tmc1^{WT/WT} mice and those injected with AAV-SaCas9-KKH-gRNA-4.2(FIGS. 9F, 9G). In contrast, uninjected Tmc1^{Bth/WT} animals showed significant hair cell loss in all regions (FIGS. 3G, 3H). In the low-frequency (8 kHz) apex, many hair cells were missing, and in the 16 and 32 kHz regions, essentially all hair cells were absent. In contrast, injected Tmc1^{Bth/WT} animals showed normal sensory epithelia in the 8 and 16 kHz regions (FIGS. 3G, 3H), with minimal hair cell loss. In the basal region (32 kHz) inner hair cells, but not outer hair cells survived (FIGS. 3G, 3H). Mean ABR thresholds were correlated with the percent of surviving hair cells at the low frequency end of the cochlea (FIG. 10C).

### Example 6: Preservation of Normal Hair Bundle Morphology

Hair bundle morphology was evaluated with scanning electron microscopy, in Bth and WT hair cells. In uninjected Tmc1^{WT/WT} animals at 24 weeks of age, inner and outer hair cells showed classical staircase organization of hair bundles (FIG. 3I). In contrast, surviving hair cells from Tmc1^{Bth/WT} animals showed significant bundle disorganization. Hair cells from SaCas9-KKH-injected mice, however, showed preservation of normal hair bundle morphology in both inner and outer hair cells (FIG. 3I). These results are concordant with the ABR data, which showed robust preservation of thresholds at low frequencies (8 and 16 kHz), but less restoration at high frequencies (32 kHz). Together, these results suggest robust therapeutic benefit of AAV-SaCas9-KKH-gRNA-4.2 injection in Bth mice.

### Example 7: Human Haploid Cells

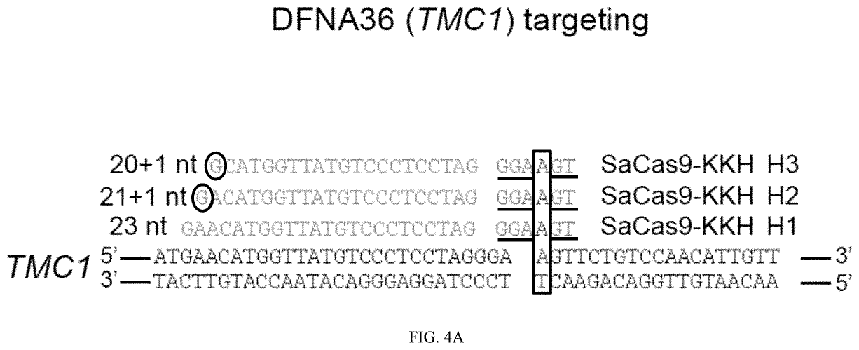

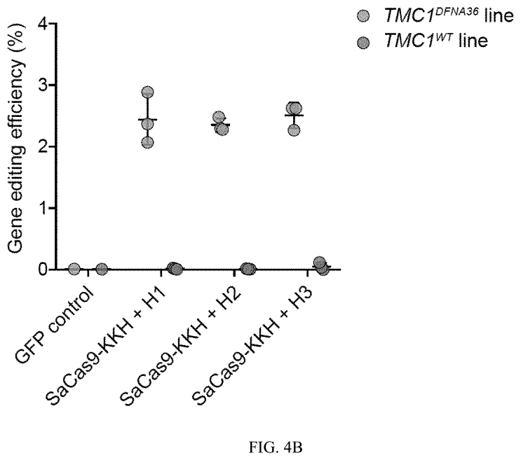

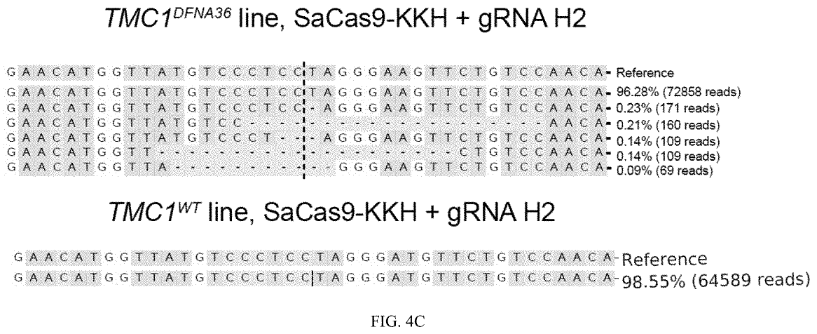

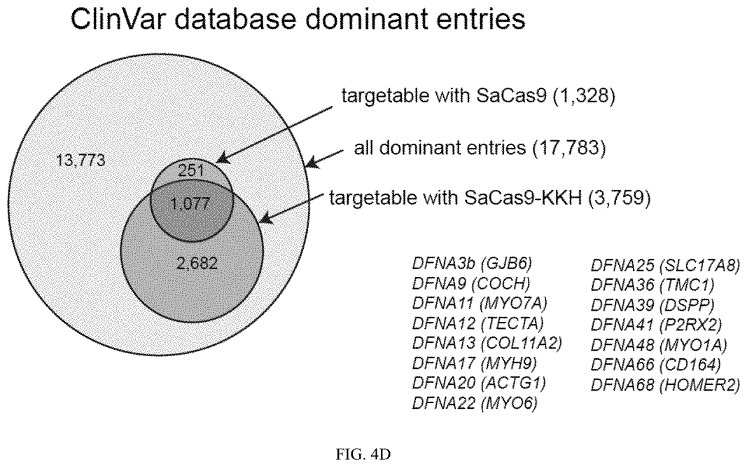

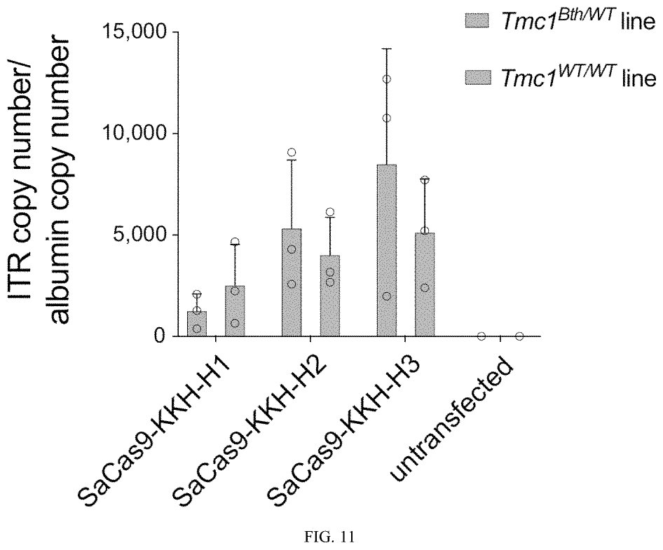

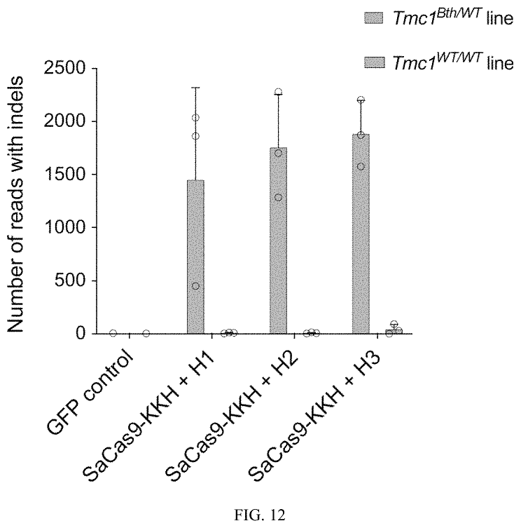

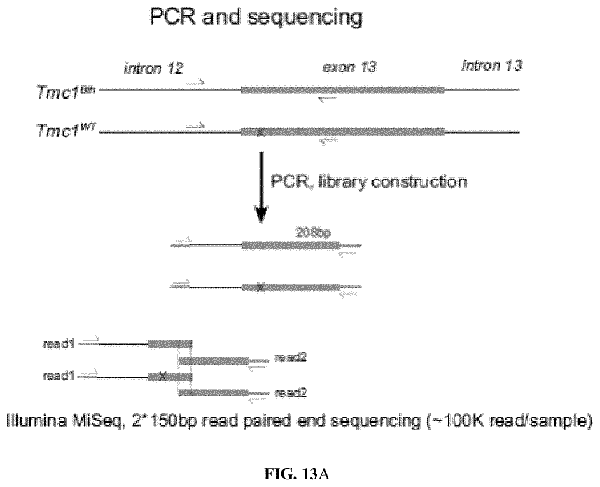

To validate the strategy for targeting the human p.M418K mutation, a haploid human cell line was generated containing the p.M418K mutation in TMC1 (c.T1253A). Tmc1^{DFNA36} and TMC1^{WT} cells were transfected with SaCas9-KKH and 3 different gRNAs targeting the mutant allele (FIG. 4A). Transfection efficiency was similar between the two lines (FIG. 11). Targeted deep sequencing of TMC1 revealed indel formation in the Tmc1^{DFNA36} line, but no indel formation in the TMC^{WT} line (FIG. 4B and FIG. 12) suggesting specific disruption of the mutant allele. The most common indel event was a single nucleotide deletion, but larger deletions were also observed (FIG. 4C). These results suggest that the strategy translates to human cells and that allele-specific targeting of dominant mutations holds promise for preventing dominant hearing loss.

### Example 8: SaCas9-KKH PAM is Present in Other Dominant Mutations

In addition to the TMC1 p.M418K mutation (DFNA36), 15 other dominant mutations in deafness genes that are targetable with SaCas9-KKH were identified (FIG. 4D and Table 1). All known dominant human mutations for specific PAM targeting using SaCas9 and SaCas9-KKH were analyzed. SaCas9 has a unique PAM requirement of ‘GRRT’, while SaCas9-KKH has a PAM requirement only of ‘RRT’. Of 17,783 dominant entries in the ClinVar database, the SaCas9 GRRT PAM site was evident in 1,328 variants (7.5%) (FIG. 4D), while the SaCas9-KKH PAM site is able to distinguish mutant from wild-type for 3,759 dominant alleles (21.1%) (FIG. 4D).

TABLE 1
Dominant deafness variants potentially targetable with SaCas9-KKH

| Deafness | | | | | | | |
|---|---|---|---|---|---|---|---|
| locus | OMIM Disease | SNP ID | WT | Variant | Protein | Gene | Link |
| DFNA11 | .0015 DEAFNESS, | RS = 121965084 | CAATG | CATTG | ASN458ILE | MYO7A | www.omim.org/entry/ |
| | AUTOSOMAL DOMINANT 11 | | | | | | 276903#0015 |
| DFNA12 | .0001 DEAFNESS, | RS = 281865415 | AGCTC | AGTTC | GLY1824ASP | TECTA | www.omim.org/entry/ |
| | AUTOSOMAL DOMINANT 12 | | | | | | 602574#0001 |
| DFNA13 | .0006 DEAFNESS, | RS = 121912947 | GCGCC | GCACC | ARG549CYS | COL11A2 | www.omim.org/entry/ |
| | AUTOSOMAL DOMINANT 13 | | | | | | 120290#0005 |
| DFNA17 | .0008 DEAFNESS, | RS = 80338828 | GGCGG | GGTGG | ARG705HIS | MYH9 | www.omim.org/entry/ |
| | AUTOSOMAL DOMINANT 17 | | | | | | 160775#0008 |
| DFNA20 | .0002 DEAFNESS, | RS = 104894544 | TCTTC | TCATC | LYS118MET | ACTG1 | www.omim.org/entry/ |
| | AUTOSOMAL DOMINANT 20 | | | | | | 102560#0002 |
| DFNA22 | .0001 DEAFNESS, | RS = 121912557 | GTGTT | GTATT | CYS442TYR | MYO6 | www.omim.org/entry/ |
| | AUTOSOMAL DOMINANT 22 | | | | | | 600970#0001 |
| DFNA22 | .0006 DEAFNESS, | RS = 121912561 | AACGA | AATGA | ARG849TER | MYO6 | www.omim.org/entry/ |
| | AUTOSOMAL DOMINANT 22 | | | | | | 600970#0006 |
| DFNA25 | .0001 DEAFNESS, | RS = 121918339 | GGCAC | GGTAC | ALA211VAL | SLC17A8 | www.omim.org/entry/ |
| | AUTOSOMAL DOMINANT 25 | | | | | | 607557#0001 |
| DFNA36 | .0007 DEAFNESS, | RS = 786201027 | GATGT | GAAGT | MET418LYS | TMC1 | www.omim.org/entry/ |
| | AUTOSOMAL DOMINANT 36 | | | | | | 606706#0007 |
| DFNA39 | .0004 DEAFNESS, | RS = 121912987 | AGGTT | AGTTT | VAL18PHE | DSPP | www.omim.org/entry/ |
| | AUTOSOMAL DOMINANT | | | | | | 125485#0004 |
| | NONSYNDROMIC | | | | | | |
| | SENSORINERAL 39, | | | | | | |
| | WITH DENTINOGENESIS | | | | | | |
| | IMPERFECTA 1 | | | | | | |
| DFNA3b | .0001 DEAFNESS, | RS = 104894414 | GACGC | GATGC | THR5MET | GJB6 | www.omim.org/entry/ |
| | AUTOSOMAL DOMINANT 3B | | | | | | 604418#0001 |
| DFNA41 | .0001 DEAFNESS, | RS = 587777692 | ACGTA | ACTTA | VAL60LEU | P2RX2 | www.omim.org/entry/ |
| | AUTOSOMAL DOMINANT 41 | | | | | | 600844#0001 |
| DFNA48 | .0004 RECLASSIFIED- | RS = 61753849 | GACAT | GAAAT | GLU385ASP | MYO1A | www.omim.org/entry/ |
| | VARIANT OF UNKNOWN | | | | | | 601478#0004 |
| | SIGNIFICANCE | | | | | | |
| DFNA66 | .0001 DEAFNESS, | RS = 876661402 | TCGTT | TCATT | ARG192TER | CD164 | www.omim.org/entry/ |
| | AUTOSOMAL DOMINANT 66 | | | | | | 603356#0001 |
| DFNA68 | .0001 DEAFNESS, | RS = 864309524 | GCCGT | GCGGT | ARG185PRO | HOMER2 | www.omim.org/entry/ |
| | AUTOSOMAL DOMINANT 68 | | | | | | 604799#0001 |
| DFNA9 | .0001 DEAFNESS, | RS = 121908927 | AGTAT | AGGAT | VAL66LGY | COCH | www.omim.org/entry/ |
| | AUTOSOMAL DOMINANT 9 | | | | | | 603196#0001 |
| DFNA9 | .0005 DEAFNESS, | RS = 121908930 | CATCC | CAACC | ILE109ASN | COCH | www.omim.org/entry/ |
| | AUTOSOMAL DOMINANT 9 | | | | | | 603196#0005 |
| DFNA9 | .0006 DEAFNESS, | RS = 12190831 | CTGCT | CTACT | ALA119THR | COCH | www.omim.org/entry/ |
| | AUTOSOMAL DOMINANT 9 | | | | | | 603196#0006 |

The results reported herein were generated using the following methods and materials.

### Plasmids and Cloning

Table 2 provides information on the specifics of the CRISPR/Cas9 plasmids used in the above examples. Table 3 shows the sequences of the gRNAs and the Cas9 plasmids used together with the gRNA vectors. 11 different gRNAs were tested with SpCas9 (FIG. 5A) that targeted the Tmc1^{Bth} allele and differed in their length and distance between the mutation and PAM site.

TABLE 2
Specification of plasmids used in this study

| Plasmid | Specification | Origin (reference) |
|---|---|---|
| pX458 | U6-sgRNA-CMV-3xFLAG-NLS(SV40)- | Addgene ID: 48138, Ran et al. 2013 |
| | SpCas9(BB)-NLS(nucleoplasmin)-T2A-GFP-bGHpA | |
| MLM3636 | U6-sgRNA | Addgene ID: 43860 |
| BPK3258 | CMV-T7-eSpCas9(1.1)(K848A, K1003A, | Addgene ID: 101176, Chen et al. |
| | R1060A)-NLS(SV40)-3xFLAG | (derived from Slaymaker et al.) |
| BPK4410 | CMV-T7-HypaCas9- (N692A, M694A, | Addgene ID: 101178, Chen et al. |
| | Q695A, H698A)-NLS(SV40)-3xFLAG | |
| VP12 | CMV-T7-SpCas9-HF1(N497A, R661A, | Addgene ID: 72247, |
| | Q695A, Q926A)-NLS(SV40)-3xFLAG | Kleinstiver BP et al. |
| pBG201 | AAV-CMV-NLS(SV40)- | Derived from pX601, |
| | SaCas9(E782K/N968K/R1015H)- | Addgene ID: 61591, Ran et al 2015 |
| | NLS(nucleoplasmin)-3xHA-bGHpA0-U6-Bsal-sgRNA | |

TABLE 3
gRNA sequences and usage

| gRNA ID | Cas9 | gRNA plasmid | Cas9 plasmid | gRNA sequence¹ + PAM (5′-3′) |
|---|---|---|---|---|
| 1.1 | SpCas9 | pX458 | pX458 | GGGTGGGACAGAACTTCCCCAGG |
| 1.1 | eSpCas9(1.1) | MLM3636 | BPK3258 | GGGTGGGACAGAACTTCCCCAGG |
| 1.1 | HypaCas9 | MLM3636 | BPK4410 | GGGTGGGACAGAACTTCCCCAGG |
| 1.1 | SpCas9-HF1 | MLM3636 | VP12 | GGGTGGGACAGAACTTCCCCAGG |
| 1.2 | SpCas9 | pX458 | pX458 | GGTGGGACAGAACTTCCCCAGG |
| 1.3 | SpCas9 | pX458 | pX458 | GTGGGACAGAACTTCCCCAGG |
| 1.4 | SpCas9 | pX458 | pX458 | GGGACAGAACTTCCCCAGG |
| 2.1 | SpCas9 | pX458 | pX458 | GTGGGACAGAACTTCCCCAGGAGG |
| 2.2 | SpCas9 | pX458 | pX458 | GGGACAGAACTTCCCCAGGAGG |
| 2.3 | SpCas9 | pX458 | pX458 | GGACAGAACTTCCCCAGGAGG |
| 2.4 | SpCas9 | pX458 | pX458 | GACAGAACTTCCCCAGGAGG |
| 3.1 | SpCas9 | pX458 | pX458 | GTGGTAATGTCCCTCCTGGGGAAG |
| 3.2 | SpCas9 | pX458 | pX458 | GGTAATGTCCCTCCTGGGGAAG |
| 3.3 | SpCas9 | pX458 | pX458 | GTAATGTCCCTCCTGGGGAAG |
| 4.1 | SaCas9-KKH | pBG201 | pBG201 | GAACATGGTAATGTCCCTCCTGGGGAAGT |
| 4.2 | SaCas9-KKH | pBG201 | pBG201 | GACATGGTAATGTCCCTCCTGGGGAAGT |
| 4.3 | SaCas9-KKH | pBG201 | pBG201 | GCATGGTAATGTCCCTCCTGGGGAAGT |
| WT | SpCas9 | pX458 | pX458 | GGGTGGGACAGAACATCCCCAGG |
| H1 | SaCas9-KKH | pBG201 | pBG201 | GAACATGGTTATGTCCCTCCTAGGGAAGT |
| H2 | SaCas9-KKH | pBG201 | pBG201 | GACATGGTTATGTCCCTCCTAGGGAAGT |
| H3 | SaCas9-KKH | pBG201 | pBG201 | GCATGGTTATGTCCCTCCTAGGGAAGT |

Non-matching 5′ G nucleotides are marked underlined

For gRNAs 1.1-1.4 and 2.1-2.4, a PAM site was adjacent to the mutation. gRNA 1.1 is identical to the Tmc1-mut3 gRNA in the study of Gao et al., Nature, 553: 217-21 (2018). In the case of gRNAs 3.1-3.3, an AAG PAM site created by the mutation was used in order to specifically recognize the mutant allele, as it has been shown that SpCas9 can also cleave at

NAG PAM sites with somewhat lower efficiency. Several truncated gRNAs were also used because previous studies reported enhanced specificity (Fu, Y. et al., Nat. Biotechnol. 32: 279-84 (2014)). One gRNA specific for the Tmc1WT allele was synthesized as a control. gRNA 1.1 was used (FIG. 5A) in combination with eSpCas9(1.1), HypaCas9 or SpCas9-HF1 (FIG. 5A). gRNA 2.4 was not used in these experiments, as it has been shown that truncated gRNAs substantially decrease on-target activity of high fidelity Cas9 enzymes. pBG201 (AAV-CMV-NLS(SV40)-SaCas9 (E782K/N968K/R1015H)-NLS(nucleoplasmin)-3xHA-bGHpA0-U6-BsaI-sgRNA) was created by synthesizing a gene fragment of the SaCas9-KKH and cloning into the pX60125 backbone using FseI (NEB) and CsiI (Thermo Scientific). Whole plasmid sequencing (MGH DNA Core, Cambridge, Mass., USA) was used to verify the sequence of pBG201. To clone gRNAs into pX458, MLM3636 and pBG201, Fast Digest BpiI (Thermo Scientific), BsmbI (NEB), and BsaI, respectively, were used. 3 different gRNAs (4.1, 4.2 and 4.3) were used with SaCas9-KKH (FIG. 5A). The correct gRNA inserts were sequenced using a U6 sequencing primer: 5′-GACTATCATATGCTTACCGT-3′.

### Cell Culture, Transfection and Sorting

Mouse primary dermal fibroblasts were established from neonatal C57BL/6 Tmc1WT/WT and Tmc1Bth/WT animals. Briefly, after euthanasia, a small amount of skin was dissected into small pieces and washed with PBS. Next, cells were treated at 37° C. with 1 mg/ml collagenase I (Worthington) for 30 minutes followed by 0.05% trypsin-EDTA treatment for 15 minutes. Cells were cultured in 10% fetal bovine serum containing DMEM (Gibco) supplemented with lx penicillin/streptomycin (Gibco). Cell lines were validated by Sanger sequencing (see below). Mycoplasma screening (MycoAlert, Lonza, Basel) was performed regularly and before transfection experiments. For transfection of fibroblasts, Nucleofection (Lonza) (CZ-167 program, P2 Primary Cell 4D-Nucleofector X Kit) was used. Every transfection reaction was performed in duplicate and experiments were performed on at least two separate occasions. Four days after transfection of pX458 plasmids, cells were sorted based on GFP fluorescence using a FACS Aria Cell Sorter (BD), and genomic DNA was isolated and analyzed by Sanger sequencing and targeted deep sequencing (see below). In the case of high fidelity SpCas9s (eSpCas9(1.1), HypaCas9, SpCas9-HF1) and SaCas9-KKH transfections, cells were not sorted, but genomic DNA was isolated from all cells 4 days after transfection for deep-sequencing analysis.

### Mouse Genomic DNA Isolation and PCR

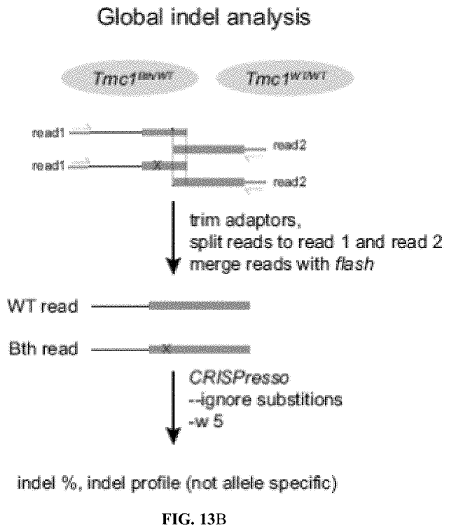

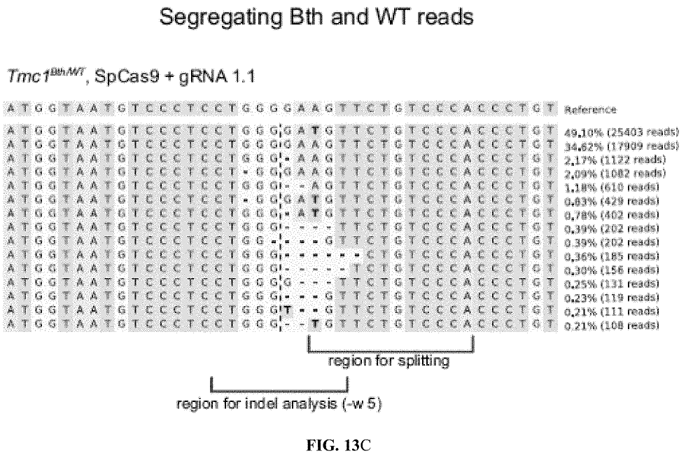

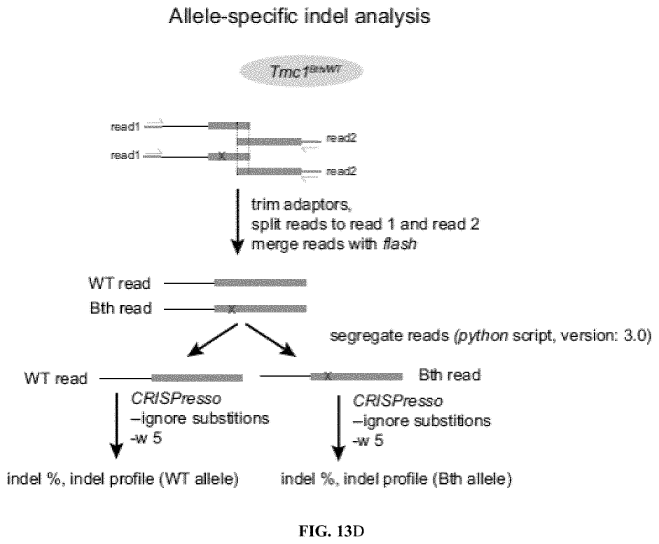

Genomic DNA was isolated from fibroblasts 4 days after transfection using a Qiagen Blood and Tissue Kit (Qiagen). In the case of cochlear tissue, organs were harvested at different ages (FIG. 2C) and dissociated with lx collagenase I/5x dispase (Gibco) for 40 minutes in Cell Dissociation Buffer (Gibco), as described previously (Scheffer, D. I., et al., J. Neurosci. 35: 6366-80 (2015), the contents of which are incorporated herein by reference in their entirety). Organs were further dissociated by passing through a 20-gauge needle 10 times. DNA and RNA from cochlear tissue were isolated using a Qiagen AllPrep DNA/RNA micro kit. To amplify the Tmc1 gene, the following primers were used: 5′-TAAAGGGACCGCTCTGAAAA-3′ (forward) and 5′-CCATCAAGGCGAGAATGAAT-3′ (reverse). To amplify Tmc1 message, 5′-CCATCAAGGCGAGAATGAAT-3′ (forward) and 5′-ACCTCATCTTTTGGGCTGTG-3′ (reverse) primers were used. PCR products were visualized on a 1% agarose gel using GelRed (Thermo Fisher) and purified on a column (PCR Purification Kit, Qiagen). For Sanger sequencing, 200-500 ng genomic DNA was used as template, and for targeted deep sequencing 500-1400 ng genomic DNA was used. Sequencing was performed at the MGH DNA Core (Sanger-sequencing and CRISPR-sequencing service). Paired-end reads (150 bp) were generated on an Illumina MiSeq platform with a 100K read depth per sample (FIG. 13A).

### Sanger Sequencing and TIDE Analysis

Sanger sequencing was performed in the MGH DNA Core. Sequence traces were analyzed by deconvolution (TIDE, Tracking Indels by Decomposition, Desktop genetics, UK). Aberrant sequences were quantified downstream of the CRISPR cut site. Analysis was performed on forward versus reverse traces and efficiency was averaged.

### Targeted Deep Sequencing Data Analysis

CRISPR-induced indels were analyzed by CRISPResso using two separate methods (FIGS. 13B and 13D, see below). During quantification, substitutions (only insertions and deletions were quantified) were ignored and indels outside of a 10 bp window of the CRISPR cut site were disregarded.

### Global CRISPResso Indel Analysis

To analyze CRISPR action on both Tmc1^{Bth} and Tmc1WT alleles, the fastq files were subjected to CRISPResso analysis without segregating them to mutant and WT reads (FIG. 13B). Briefly, reads were split to read 1 and read 2 then merged using flash v1.2.11 (parameters: min overlap: 4, max overlap: 126, max mismatch density: 0.250000, allow “outie” pairs: true, cap mismatch quals: false, combiner threads: 8, input format: fastq, phred_offset=33, output format: fastq, phred_offset=33). Next, CRISPResso was run with the following parameters: CRISPResso-r1 <fastq_file>--split_paired_end-w 5-c <protein_coding_sequence>--ignore_substitutions-a <amplicon sequence>-g <gRNA sequence>.

Allele-Specific CRISPResso Indel Analysis

To analyze CRISPR action on Tmc1Bth and Tmc1WT alleles separately, fastq files were first split into read 1 and read 2, and then merged using flash v1.2.11, as described above. The reads from heterozygous samples were segregated based on the presence of wild-type sequence (“TGGGACAGAACA” and its reverse complement “TGTTCTGTCCCA”) and mutant sequence (“TGGGACAGAACT” and its reverse complement “AGTTCTGTCCCA”; mutation site is underlined) using a custom Python script (version 3.4.2) used previously (György, B. et al., Mol. Ther.-Nucleic Acids 11: 429-40 (2018), the contents of which are incorporated herein by reference in their entirety). Reads were segregated based on a sequence downstream of the projected CRISPR cut site so that indels have minor influence on the segregation (FIG. 13C). After segregation, CRISPResso was run separately on Tmc1Bth and Tmc1WT reads with the following paramters: -r1 <fastq_file>-w 5 -c <protein_coding_sequence>--ignore_substitutions -a <amplicon_sequence>-g <gRNA_sequence>.

For mRNA analysis, the reads were first merged with flash as described above. CRISPResso analysis was performed similarly to global indel analysis (see above). To quantify intact, non-edited reads, the following sequences were used: 5′-CATCCCCAGGAGGG-3′ and 5′-CCCTCCTGGGGATG-3′ for WT reads, and 5′-CTTCCCCAGGAGGG-3′ and 5′-CCCTCCTGGGGAAG-3′ for mutant reads.

### Off-Target Analysis

To detect genome-wide Cas9 nuclease activity, a GUIDE-Seq assay was performed in fibroblasts. Briefly, 2 μg of Cas9-2A-GFP-U6-gRNA-2.4 or 2 μg of pAAV-CMV-SaCas9-KKH-U6-gRNA-4.2 along with 50 pmol annealed GUIDE-Seq oligo (forward:/5Phos/G*T*TTAATTGAGTTGTCATATGTTAATAACGGT*A*T, reverse:/5Phos/A*T*ACCGTTATTAACATATGACAACTCAATTAA*A*C, stars indicate thioate bonds) were transfected into Tmc1Bth/WT fibroblasts using electroporation (see above). Four days after transfection, genomic DNA was isolated with a Qiagen DNA Blood and Tissue kit, and a library was constructed as described previously (Tsai, S. Q. et al., Nat. Biotechnol. 33: 187-98 (2015), the contents of which are incorporated herein by reference in their entirety). Sequencing was performed on an Illumina MiSeq machine. As a control, GUIDE-Seq oligo was transfected without CRISPR/Cas9 plasmids. GUIDE-Seq data were analyzed with the guideseq pipeline v1.1b4 (github.com/aryeelab/guideseq) using mm10 as the reference mouse genome.

### AAV Vector Production

AAV vectors were produced by the Boston Children's Hospital Viral Core (Boston, MA, USA). Plasmid containing SaCas9-KKH and gRNA 4.2 was sequenced before packaging (MGH DNA Core, complete plasmid sequencing) into AAV2/Anc8028. Vector titer was 4.8×10¹⁴ gc/ml as determined by qPCR specific for the inverted terminal repeat of the virus.

### Inner Ear Injections

Inner ears of Tmc1Bth/WT or Tmc1WT//WT mouse pups were injected at P1 with 1 μl of Anc80-AAV-CMV-SaCas9-KKH-U6-gRNA-4.2 virus at a rate of 60 nl/min. Pups were anesthetized using hypothermia exposure in ice water for 2-3 min. Upon anesthesia, a post-auricular incision was made to expose the otic bulla and visualize the cochlea. Injections were made manually with a glass micropipette. After injection, a suture was used to close the skin cut. Then, the injected mice were placed on a 42° C. heating pad for recovery. Pups were returned to the mother after they fully recovered within ˜10 min. Standard post-operative care was applied after surgery. Sample sizes for in vivo studies were determined on a continuing basis to optimize the sample size and decrease the variance. At P5 to P7, organs of Corti were excised from injected ears. Organ of Corti tissues were incubated at 37° C., 5% CO₂ for 8-10 days, the tectorial membrane was removed immediately before electrophysiology recording.

### Hair Cell Electrophysiology

Mechanotransduction currents were recorded from cochlear IHCs and OHCs at P14-16. Organ of Corti tissues were bathed in external solution containing: 140 mM NaCl, 5.8 mM KCl, 0.7 mM NaH₂PO₄, 10 mM HEPES, 1.3 mM CaCl₂, 0.9 mM MgCl₂, 5.6 mM glucose, and vitamins and essential amino acids (Thermo Fisher Scientific, Waltham, Mass.), adjusted to pH 7.4 with NaOH, ˜310 mmol/kg. Recording electrodes were pulled from R6 capillary glass (King Precision Glass). The intracellular solution contained: 140 mM CsCl, 5 mM EGTA, 5 mM HEPES, 2.5 mM Na₂-ATP, 0.1 mM CaCl₂, and 3.5 mM MgCl₂, and was adjusted to pH 7.4 with CsOH, ˜285 mmol/kg. Mechanotransduction currents were recorded under whole-cell voltage-clamp configuration using an Axopatch 200B (Molecular Devices) amplifier. Cells were held at −80 mV for all electrophysiology recordings. Data were low-pass filtered at 5 kHz (Bessel filter), then sampled at 20 kHz with a 16-bit acquisition board (Digidata 1440A). Data were corrected for a −4 mV liquid junction potential in standard extracellular solutions. Cochlea IHC and OHC bundles were deflected using stiff glass probes mounted on a PICMA chip piezo actuator (Physik Instruments) driven by an LPZT amplifier (Physik Instruments) and filtered with an 8-pole Bessel filter at 40 kHz to eliminate residual pipette resonance. Fire-polished stimulus pipettes with 3-5 μm tip diameter were designed to fit into the concave aspect of hair cell bundle as previously described (Stauffer and Holt, 2007). Hair bundle deflections were monitored using a C2400 CCD camera (Hamamatsu, Japan).

### Hearing Tests

ABR and DPOAE measurements were recorded using the EPL Acoustic system (Massachusetts Eye and Ear, Boston). Stimuli were generated with 24-bit digital I-O cards (National Instruments PXI-4461) in a PXI-1042Q chassis, amplified by a SA-1 speaker driver (Tucker-Davis Technologies, Inc.), and delivered from two electrostatic drivers (CUI CDMG15008-03A) in a custom acoustic system. An electret microphone (Knowles FG-23329-P07) at the end of a small probe tube was used to monitor ear-canal sound pressure. ABRs and DPOAEs were recorded from mice during the same session. Mice were anesthetized with intraperitoneal injection of xylazine (5-10 mg/kg) and ketamine (60-100 mg/kg), and the base of the pinna was trimmed away to expose the ear canal. Three subcutaneous needle electrodes were inserted into the skin, including a) dorsally between the two ears (reference electrode); b) behind the left pinna (recording electrode); and c) dorsally at the rump of the animal (ground electrode). Additional aliquots of ketamine (60-100 mg/kg i.p.) were given throughout the session to maintain anesthesia if needed. DPOAEs were recorded first. F1 and f2 primary tones (f2/f1=1.2) were presented with f2 varied between 5.6 and 32.0 kHz in half-octave steps and L1−L2=10 dB SPL. At each f2, L2 was varied between 10 and 80 dB in 10 dB increments. DPOAE threshold was defined from the average spectra as the L2-level eliciting a DPOAE of magnitude 5 dB above the noise floor. The mean noise floor level was under 0 dB across all frequencies. ABR recordings were then recorded, with stimuli of broadband “click” tones as well as the pure tones between 5.6 and 32.0 kHz in half-octave steps, all presented as 5 ms tone pips. The responses were amplified (10,000 times), filtered (0.1-3 kHz), and averaged with an analog-to-digital board in a PC based data-acquisition system (EPL, Cochlear function test suite, MEE, Boston). Across various trials, the sound level was raised in 5 to 10 dB steps from 0 to 110 dB sound pressure level (decibels SPL). At each level, 512 responses were averaged (with stimulus polarity alternated) after “artifact rejection”. Threshold was determined by visual inspection of the appearance of Peak 1 relative to background noise. Data were analyzed and plotted using Origin-2015 (OriginLab Corporation, MA). Thresholds averages±standard deviations are presented unless otherwise stated. The majority of these experiments were not performed under blind conditions.

### Confocal Microscopy

The temporal bones of 24-week-old adult mice were harvested, cleaned, and placed in 4% PFA for 1 hour, followed by decalcification for 24 to 36 h with 120 mM EDTA (pH=7.4). The sensory epithelium was then dissected and remained in PBS until staining. Tissues were permeabilized with 0.01% Triton X-100 for one hour, blocked with 2.5% NDS and 2.5% BSA in 0.01% Triton X-100 for one hour, and then incubated with anti-MYO7A primary antibody (Proteus Biosciences) overnight (1:500 dilution). Tissues were then washed and counterstained with phalloidin for 2-3 hours. Images were acquired on a Zeiss LSM 800 laser confocal microscope. Full cochlear maps were reconstructed in Adobe Photoshop and tonotopically mapped using an ImageJ plugin.

### Scanning Electron Microscopy

The temporal bones of 24-week-old adult mice were harvested, and cleaned temporal bones were placed in 2.5% glutaraldehyde in 0.1 M cacodylate buffer (EMS) supplemented with 2 mM CaCl₂ for 45 minutes. Whole-mount tissues were dissected in distilled water and then dehydrated over the course of 4 hours to pure ethanol. Tissues were critical-point dried (Autosamdri-815, series A, Tousimis) and mounted on carbon tape attached to SEM specimen stubs. The mounted tissues were coated with 4 nm of platinum (Leica EM ACE600) and then imaged at 5 kV with a scanning electron microscope (Hitachi S-4700 FESEM).

### ClinVar Database Analysis

The ClinVar database from Apr. 25, 2019 was downloaded from ftp.ncbi.nlm.nih.gov/pub/clinvar/vcf_GRCh38/archive_2.0/2019/clinvar 20190325.vcf.gz. The database was filtered for ‘dominant’ diseases, resulting in 17,783 entries. The possibility of generating a PAM site from single nucleotide mutations was analyzed for the SaCas9 (GRRT) and SaCas9-KKH (RRT) recognition motifs. The GRCh38 human reference genome was queried for 7-nt or 5-nt sequences surrounding the site of interest, i.e., 3 (GRRT) or 2 (RRT) nucleotides on either side of the mutation. These sequences were analyzed with a sliding window of length 4 (GRRT) or 3 (RRT) nucleotides. Entries that had a putative PAM site in both the ‘variant’ nucleotide string and the ‘reference’ (i.e. wild-type) string were excluded from further analysis. The same procedure was used for the reverse complement strand. The resulting databases (all dominant entries, dominant entries with PAM site formation for SaCas9, and dominant entries with PAM site formation for SaCas9-KKH recognition) are uploaded as Supplementary Tables 3-5. All ClinVar entries were analyzed without filtering from dominant diseases. This analysis was done, as several dominant variants are not annotated as ‘dominant’ in the ClinVar database.

### TMC1 Gene Inactivation in Human Haploid Cells

A human cell line carrying the equivalent of the Beethoven mutation in the human TMC1 gene (ATG to AAG mutation, encoding the M418K amino acid substitution) was engineered from the HAP1 parental cell line derived from the KBM-7 haploid cells (Horizon Genomics GmbH, Vienna, Austria)29. Briefly, a T to A point mutation was introduced in the exon 16 of the TMC1 gene (ENSG00000165091; genomic location: chr9: 72,791,914) to generate the TCCCTCCTAGGGAAGTTC sequence. For insertion of the mutation by gene editing, the HAP1 cell line was modified with the CRISPR/Cas9 nuclease using two guide RNA sequences (5′-CATCGCTTTGAAATGGCTAC-3′ and 5′-AACCATGTTCATCTACAAGG-3′) and a 1 kb donor template encompassing TMC1 exon 6 and which contained the T to A Beethoven mutation. The genetic identity of the cells was verified by Sanger sequencing of a PCR amplicon.

The parental and HAP1 cell lines were cultured as monolayer at 37° C. in a humidified atmosphere with 5% CO₂, using IMDM medium plus GlutaMAX (Gibco) supplemented with 10% FBS, 100 U/ml penicilin and 100 μg/ml streptomycin. Cells were passaged every 2-3 days when reaching 70-75% confluency. For transfection, the cells were grown in 6-well plates at a 70% confluency. One day later, cells were transfected with 2.5 μg pDNA using Lipofectamine 3000 (ThermoFisher), following manufacturer's instructions. Two days after transfection, the cells were collected by trypsinization, and the pellet was stored at −20° C. Total genomic DNA was extracted from the cells with the NucleoSpin® Tissue kit (Macherey-Nagel AG, Switzerland). A PCR amplicon was amplified for next-generation sequencing, using the Phusion High-Fidelity DNA Polymerase (ThermoFisher). For TMC1DFNA36 cells, the primers used were 5′-AGCCTAGCTCAGAATCTTCCA-3′ and 5′-AAAATGCGTCCCAGTAGCCA-3′. For TMCWT cells, the 5′-AAAATGCGTCCAAGTAGCCA-3′ was used due to a point mutation in the primer binding region. The PCR protocol was based on manufacturer's instruction, with 35 cycles (5 s at 98° C.; 20 s at 59° C.; 15 s at 72° C.). The PCR product was visualized on a 2% agarose gel and purified with the PCR clean-up and gel extraction kit (Macherey-Nagel AG, Switzerland).

Next-generation sequencing was performed by the Massachusetts General Hospital DNA Core facility.

To verify transfection efficacy in each sample, TaqMan real-time PCR was used to quantify the number of plasmid copies of the sequence contained in the AAV inverted terminal repeats using the following primers: forward: 5′-GGA ACC CCT AGT GAT GGA GTT-3′; reverse: 5′-CGG CCT CAG TGA GCG A-3′; probe: 5′-FAM-CAC TCC CTC TCT GCG CGC TCG-BHQ1-3′. The amount of cellular gDNA was quantified using a set of primers specific for the human albumin gene: forward: 5′-TGA AAC ATA CGT TCC CAA AGA GTT T-3′; reverse: 5′-CTC TCC TTC TCA GAA AGT GTG CAT AT-3′; probe: 5′-FAM-TGC TGA AAC ATT CAC CTT CCA TGC AGA-BHQ1-3′. Absolute number of copies were determined according to standards and used to calculate the number of plasmid copies per cell.

### Statistical Analysis

GraphPad Prism 7.0 for Mac OS and OriginPro (2015) were used for statistical analysis. To compare means, an unpaired two tailed t-test (after Shapiro-Wilk normality testing) was used; to compare multiple groups, ANOVA followed by Tukey's post-hoc test (to compare every mean to every other mean) or Dunnett's test (to compare every mean to a control group mean) was used. p values <0.05 were accepted as significant.

### Other Embodiments

From the foregoing description, it will be apparent that variations and modifications may be made to the invention described herein to adopt it to various usages and conditions. Such embodiments are also within the scope of the following claims.

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

All patents and publications mentioned in this specification are herein incorporated by reference to the same extent as if each independent patent and publication was specifically and individually indicated to be incorporated by reference.

What is claimed is:

1. A method for allele specific gene editing, the method comprising contacting a double stranded polynucleotide comprising a wild-type allele and a mutant allele with a guide RNA that binds the alleles and a Cas9 polypeptide with a PAM site selective for the mutant allele, such that indels are selectively induced in the mutant target allele.

2. A method for the allele-specific disruption of a dominant mutation, the method comprising contacting a double stranded polynucleotide comprising a wild-type allele and a mutant allele with a guide RNA that binds the alleles and a Cas9 nuclease with a PAM site selective for the mutant allele, such that indels are selectively induced in the mutant allele.

3. The method of claim 1, wherein the double stranded polynucleotide is DNA.

4. The method of claim 3, wherein the DNA is genomic DNA.

5. The method of claim 1, wherein the polynucleotide is present in a cell.

6. The method of claim 5, wherein the cell is a cell in vivo or in vitro.

7. A method for the treatment of a disorder associated with a dominant mutant allele in a target gene, the method comprising:
(a) contacting a cell heterozygous for the dominant mutant allele in a target gene with a guide RNA that binds the target gene and a Cas9 nuclease with a PAM site selective for the mutant allele; and
(b) selectively inducing an indel in the mutant allele.

8. A method of treating progressive hearing loss in a subject, the method comprising
(a) contacting a cell of a subject heterozygous for a p.M418K mutation in TMC1 with a SaCas9-KKH and a guide RNA that targets TMC1; and
(b) inducing indels in the TMC1 allele comprising the p.M418K mutation, thereby treating hearing loss in the subject.

9. The method of claim 8, wherein the cell is a cell of the inner ear.

10. The method of claim 9, wherein the cell is an inner or outer hair cell.

11. The method of claim 8, wherein the administering improves or maintains auditory function in the subject.

12. The method of claim 11, wherein an improvement in auditory function is associated with preservation of hair bundle morphology and/or restoration of mechanotransduction.

13. The method of claim 1, wherein the guide RNA and the Cas9 polypeptide are encoded in a single vector.

14. The method of claim 13, wherein the vector is an adeno-associated virus vector or a lentivirus vector.

15. The method of claim 1, wherein the contacting comprises transfecting cells in the subject with a guide RNA and a polynucleotide encoding a Cas9 protein.

16. The method of claim 15, wherein the guide RNA and the Cas9 polypeptide are administered simultaneously.

17. A vector comprising a polynucleotide encoding a SaCas9-KKH polypeptide, or a fragment thereof, and a gRNA having a nucleic acid sequence complementary to a nucleic acid sequence comprising a mutation associated with DFNA36.

18. A pharmaceutical composition comprising the vector of claim 17.


SEQUENCE LISTING (135 sequences)

SEQ ID NO: 1 - DNA, 8543 bp, Artificial Sequence
  Description of Artificial Sequence Synthetic polynucleotide
ccaatgatacgcgtcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattgggtacatcgacggtatcgggggagctcgcagggtctccattttgaagcgggaggtttgaacgcgcagccgccatgccggggttttacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggctcttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtggacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaatatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcggacaggtaccaaaacaaatgttctcgtcacgtgggcatgaatctgatgctgtttccctgcagacaatgcgagagactgaatcagaattcaaatatctgcttcactcacggtgtcaaagactgtttagagtgctttcccgtgtcagaatctcaacccgtttctgtcgtcaaaaaggcgtatcagaaactgtgctacattcatcacatcatgggaaaggtgccagacgcttgcactgcttgcgacctggtcaatgtggacttggatgactgtgtttctgaacaataaatgacttaaaccaggtatgagtcggctggataaatctaaagtcataaacggcgctctggaattactcaatgaagtcggtatcgaaggcctgacgacaaggaaactcgctcaaaagctgggagttgagcagcctaccctgtactggcacgtgaagaacaagcgggccctgctcgatgccctggccatcgagatgctggacaggcatcatacccacttctgccccctggaaggcgagtcatggcaagactttctgcggaacaacgccaagtcattccgctgtgctctcctctcacatcgcgacggggctaaagtgcatctcggcacccgcccaacagagaaacagtacgaaaccctggaaaatcagctcgcgttcctgtgtcagcaaggcttctccctggagaacgcactgtacgctctgtccgccgtgggccactttacactgggctgcgtattggaggaacaggagcatcaagtagcaaaagaggaaagagagacacctaccaccgattctatgcccccacttctgagacaagcaattgagctgttcgaccggcagggagccgaacctgccttccttttcggcctggaactaatcatatgtggcctggagaaacagctaaagtgcgaaagcggcgggccggccgacgcccttgacgattttgacttagacatgctcccagccgatgcccttgacgactttgaccttgatatgctgcctgctgacgctcttgacgattttgaccttgacatgctccccgggtaaatgcatgaattcgatctagagggccctattctatagtgtcacctaaatgctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctcgaatcaagctatcaagtgccacctgacgtctccctatcagtgatagagaagtcgacacgtctcgagctccctatcagtgatagagaaggtacgtctagaacgtctccctatcagtgatagagaagtcgacacgtctcgagctccctatcagtgatagagaaggtacgtctagaacgtctccctatcagtgatagagaagtcgacacgtctcgagctccctatcagtgatagagaaggtacgtctagaacgtctccctatcagtgatagagaagtcgacacgtctcgagctccctatcagtgatagagaaggtaccccctatataagcagagagatctgttcaaatttgaactgactaagcggctcccgccagattttggcaagattactaagcaggaagtcaaggacttttttgcttgggcaaaggtcaatcaggtgccggtgactcacgagtttaaagttcccagggaattggcgggaactaaaggggcggagaaatctctaaaacgcccactgggtgacgtcaccaatactagctataaaagtctggagaagcgggccaggctctcatttgttcccgagacgcctcgcagttcagacgtgactgttgatcccgctcctctgcgaccgctagcttcgatcaactacgcagacaggtaccaaaacaagtgttctcgtcacgtgggcattaatctgattctgtttccctgcagacaatgcgagagaatgaatcagaactcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaatgacttaagccaggtatggctgccgatggttatcttccagattggctcgaggacaaccttagtgaaggaattcgcgagtggtgggctttgaaacctggagcccctcaacccaaggcaaatcaacaacatcaagacaacgctagaggtcttgtgcttccgggttacaaataccttggacccggcaacggactcgacaagggggagccggtcaacgcagcagacgcggcggccctcgagcacgacaaagcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctctttaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctctagaactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggcctctcacaaagaaggagaggaccgtttctttcctttgtctggatctttaatttttggcaaacaaggtactggcagagacaacgtggatgcggacaaagtcatgataaccaacgaagaagaaattaaaactactaacccggtagcaacggagtcctatggacaagtggccacaaaccaccagagtgcccaaactttggcggtgccttttaaggcacaggcgcagaccggttgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggtcaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaagtcgacttgcttgttaatcaataaaccgtttaattcgtttcagttgaactttggtctctgcgaagggcaattcgtttaaacctgcaggactagaggtcctgtattagaggtcacgtgagtgttttgcgacattttgcgacaccatgtggtcacgctgggtatttaagcccgagtgagcacgcagggtctccattttgaagcgggaggtttgaacgcgcagccgccaagccgaattctgcagatatcacatgtcctaggaactatcgatccatcacactggcggccgctcgactagagcggccgccaccgcggtggagctccagcttttgcggaccgaatcggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 2 - protein, 734 aa, Unknown
  Description of Unknown Anc80 sequence
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEQSPQEPDSSSGIGKKGQQPARKRLNFGQTGDSESVPDPQPLGEPPAAPSGVGSNTMAAGGGAPMADNNEGADGVGNASGNWHCDSTWLGDRVITTSTRTALPTYNNHLYKQISSQSGGSTNDNTYFGYSTPWGYFDFNRFHCHFSPRDWQRLINNNWGFRPKKLNFKLFNIQVKEVTTNDGTTTIANNLTSTVQVFTDSEYQLPYVLGSAHQGCLPPFPADVFMIPQYGYLTLNNGSQAVGRSSFYCLEYFPSQMLRTGNNFQFSYTFEDVPFHSSYAHSQSLDRLMNPLIDQYLYYLSRTQTTSGTAGNRTLQFSQAGPSSMANQAKNWLPGPCYRQQRVSKTTNQNNNSNFAWTGATKYHLNGRDSLVNPGPAMATHKDDEDKFFPMSGVLIFGKQGAGNSNVDLDNVMITNEEEIKTTNPVATEEYGTVATNLQSANTAPATGTVNSQGALPGMVWQDRDVYLQGPIWAKIPHTDGHFHPSPLMGGFGLKHPPPQILIKNTPVPANPPTTFSPAKFASFITQYSTGQVSVEIEELQKENSKRWNPEIQYTSNYNKSTNVDFAVDTNGVYSEPRPIGTRYLTRNL

SEQ ID NO: 3 - protein, 1368 aa, Streptococcus pyogenes [sequence omitted]

SEQ ID NO: 4 - DNA, 4107 bp, Streptococcus pyogenes
atggataagaaatactcaataggcttagatatcggcacaaatagcgtcggatgggcggtgatcactgatgaatataaggttccgtctaaaaagttcaaggttctgggaaatacagaccgccacagtatcaaaaaaaatcttataggggctcttttatttgacagtggagagacagcggaagcgactcgtctcaaacggacagctcgtagaaggtatacacgtcggaagaatcgtatttgttatctacaggagattttttcaaatgagatggcgaaagtagatgatagtttctttcatcgacttgaagagtcttttttggtggaagaagacaagaagcatgaacgtcatcctatttttggaaatatagtagatgaagttgcttatcatgagaaatatccaactatctatcatctgcgaaaaaaattggtagattctactgataaagcggatttgcgcttaatctatttggccttagcgcatatgattaagtttcgtggtcattttttgattgagggagatttaaatcctgataatagtgatgtggacaaactatttatccagttggtacaaacctacaatcaattatttgaagaaaaccctattaacgcaagtggagtagatgctaaagcgattctttctgcacgattgagtaaatcaagacgattagaaaatctcattgctcagctccccggtgagaagaaaaatggcttatttgggaatctcattgctttgtcattgggtttgacccctaattttaaatcaaattttgatttggcagaagatgctaaattacagctttcaaaagatacttacgatgatgatttagataatttattggcgcaaattggagatcaatatgctgatttgtttttggcagctaagaatttatcagatgctattttactttcagatatcctaagagtaaatactgaaataactaaggctcccctatcagcttcaatgattaaacgctacgatgaacatcatcaagacttgactcttttaaaagctttagttcgacaacaacttccagaaaagtataaagaaatcttttttgatcaatcaaaaaacggatatgcaggttatattgatgggggagctagccaagaagaattttataaatttatcaaaccaattttagaaaaaatggatggtactgaggaattattggtgaaactaaatcgtgaagatttgctgcgcaagcaacggacctttgacaacggctctattccccatcaaattcacttgggtgagctgcatgctattttgagaagacaagaagacttttatccatttttaaaagacaatcgtgagaagattgaaaaaatcttgacttttcgaattccttattatgttggtccattggcgcgtggcaatagtcgttttgcatggatgactcggaagtctgaagaaacaattaccccatggaattttgaagaagttgtcgataaaggtgcttcagctcaatcatttattgaacgcatgacaaactttgataaaaatcttccaaatgaaaaagtactaccaaaacatagtttgctttatgagtattttacggtttataacgaattgacaaaggtcaaatatgttactgaaggaatgcgaaaaccagcatttctttcaggtgaacagaagaaagccattgttgatttactcttcaaaacaaatcgaaaagtaaccgttaagcaattaaaagaagattatttcaaaaaaatagaatgttttgatagtgttgaaatttcaggagttgaagatagatttaatgcttcattaggtacctaccatgatttgctaaaaattattaaagataaagattttttggataatgaagaaaatgaagatatcttagaggatattgttttaacattgaccttatttgaagatagggagatgattgaggaaagacttaaaacatatgctcacctctttgatgataaggtgatgaaacagcttaaacgtcgccgttatactggttggggacgtttgtctcgaaaattgattaatggtattagggataagcaatctggcaaaacaatattagattttttgaaatcagatggttttgccaatcgcaattttatgcagctgatccatgatgatagtttgacatttaaagaagacattcaaaaagcacaagtgtctggacaaggcgatagtttacatgaacatattgcaaatttagctggtagccctgctattaaaaaaggtattttacagactgtaaaagttgttgatgaattggtcaaagtaatggggcggcataagccagaaaatatcgttattgaaatggcacgtgaaaatcagacaactcaaaagggccagaaaaattcgcgagagcgtatgaaacgaatcgaagaaggtatcaaagaattaggaagtcagattcttaaagagcatcctgttgaaaatactcaattgcaaaatgaaaagctctatctctattatctccaaaatggaagagacatgtatgtggaccaagaattagatattaatcgtttaagtgattatgatgtcgatcacattgttccacaaagtttccttaaagacgattcaatagacaataaggtcttaacgcgttctgataaaaatcgtggtaaatcggataacgttccaagtgaagaagtagtcaaaaagatgaaaaactattggagacaacttctaaacgccaagttaatcactcaacgtaagtttgataatttaacgaaagctgaacgtggaggtttgagtgaacttgataaagctggttttatcaaacgccaattggttgaaactcgccaaatcactaagcatgtggcacaaattttggatagtcgcatgaatactaaatacgatgaaaatgataaacttattcgagaggttaaagtgattaccttaaaatctaaattagtttctgacttccgaaaagatttccaattctataaagtacgtgagattaacaattaccatcatgcccatgatgcgtatctaaatgccgtcgttggaactgctttgattaagaaatatccaaaacttgaatcggagtttgtctatggtgattataaagtttatgatgttcgtaaaatgattgctaagtctgagcaagaaataggcaaagcaaccgcaaaatatttcttttactctaatatcatgaacttcttcaaaacagaaattacacttgcaaatggagagattcgcaaacgccctctaatcgaaactaatggggaaactggagaaattgtctgggataaagggcgagattttgccacagtgcgcaaagtattgtccatgccccaagtcaatattgtcaagaaaacagaagtacagacaggcggattctccaaggagtcaattttaccaaaaagaaattcggacaagcttattgctcgtaaaaaagactgggatccaaaaaaatatggtggttttgatagtccaacggtagcttattcagtcctagtggttgctaaggtggaaaaagggaaatcgaagaagttaaaatccgttaaagagttactagggatcacaattatggaaagaagttcctttgaaaaaaatccgattgactttttagaagctaaaggatataaggaagttaaaaaagacttaatcattaaactacctaaatatagtctttttgagttagaaaacggtcgtaaacggatgctggctagtgccggagaattacaaaaaggaaatgagctggctctgccaagcaaatatgtgaattttttatatttagctagtcattatgaaaagttgaagggtagtccagaagataacgaacaaaaacaattgtttgtggagcagcataagcattatttagatgagattattgagcaaatcagtgaattttctaagcgtgttattttagcagatgccaatttagataaagttcttagtgcatataacaaacatagagacaaaccaatacgtgaacaagcagaaaatattattcatttatttacgttgacgaatcttggagctcccgctgcttttaaatattttgatacaacaattgatcgtaaacgatatacgtctacaaaagaagttttagatgccactcttatccatcaatccatcactggtctttatgaaacacgcattgatttgagtcagctaggaggtgactga

SEQ ID NO: 5 - protein, 1087 aa, Staphylococcus aureus
MAPKKKRKVGIHGVPAAKRNYILGLDIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEENSKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKGKRPAATKKAGQAKKKKGS

SEQ ID NO: 6 - protein, 1087 aa, Artificial Sequence
  Description of Artificial Sequence Synthetic polypeptide
MAPKKKRKVGIHGVPAAKRNYILGLDIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEENSKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRKLINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYKNDLIKINGELYRVIGVNNDLLNRIEVNMIDITYREYLENMNDKRPPHIIKTIASKTQSIKKYSTDILGNLYEVKSKKHPQIIKKGKRPAATKKAGQAKKKKGS

SEQ ID NO: 7 - DNA, 964 bp, Homo sapiens
tcttcacctgtcattttcaaccagcctcagcctatctgctctgtcacaatcactactaaaatatgttcctaaattgcttgtttctagatccttccttctcatatgctcaggtgaacacatgggtgaaatttaatatggaattgaaatatgtactatgcaagatagattccttaagaaatgtttctctgatttatatgacataattgtattttactagtttacctgtccatctgtaaaactttgttttggagatttcatatattacaatgtttaagaaatatgctataatgttttgtatagtatatttcttcgtgataaccttatatactaccagtcacacgtgtttgtaaaaatctaaagagtacttttggctcctacagaatgtgtgaagttgtgaaattgtttttttgttttgttttgttttgtttttatgccccaaagatgtggagggcttcatataagagggtagatttaatgagagagagagggagagacagagagaatgataaaagaagcttaagagattattttatcttgtcaacgacattgttattgaatgtaagctgctaaacttcttagataaagtaaaacagtaaaaacaaacacacaaaacagaacagagaatcatcagacaggctgacgaacacagtacaataaagcagccagtaccgatgatcagtggacatcaatttgtcttttgggctgtagcacctgctactaattggtgcaaagcgctcaccagtcagtgcgtggtttagcgcactcagctgtctcctgtatgtgctgcgagaagcaagatagctaattgctgttgcttcagtgccagtgaaatcaacgtgctgagctaatagcgacagatagagggcagacagattcctgctagcagcttagtgttagttgcttgtggtaactaaggcaggtggcatacatctcagaacgtggagaatgatggtatgctttctga

SEQ ID NO: 8 - DNA, 1040 bp, Homo sapiens
tggtagcctccctagagacacagagctgggccggatgagtccaggcactgacgtgatccattatctttcaccttaaagagtaaaagggaaactaaagttaattacctccacgaaacaaaaaggtgccttcttgtgcttcaattacatggatatattctactagtctaaaagtatcttctcacttctttctgtcactgtgaggacttgagtcagaagaaagtttaaatacagtcattgagctggaaagagtggaaagagaagcaaagagggggaagctgtaggaaggacgaagtcacccccaagatacatggttactgcttacaccaagcaagctgccttgggaacgcttcccccgagcagccagaatgctcagcagtggaagacacctctattcctgtaggcgagtcctgggaagctggtcaatctgcaaatgccaattcccagcagtgagctcggtccacgtgtaaatcaagatttggggaaagagtagggtgggtggcatggttgacaatgtcatcagctccctcctctgactcctgtggtcgtgcccccatctactctcactcagctacaccccaccttcggatttgtgatggacgctgggtccctagtaaccacagcaagtgtctcccccgcacttcccccttccccacccccacccccacccccaaccaccaccccagcgatggagcctactctgctccaagccgccgctaagacccggagaagcggaatttcactttgaaattcccttgcctcgtgagggccggcgctgggcatgctcagtagccgcggcgctgctgctgggctgctgggctggcgcggagtccaccctgccgtctccgccttggcttctgggcgtccagaaggccaggcatttgccgcctctgagcgcttctgttccccttacccgcaacctcctactgctcttcctctctccctctcttagggaggttgaagctggtgctggtttctgtcggcgccacagactgactgctctgcaaaccccagccgaggacctgaatcccggagactagaag

SEQ ID NO: 9 - DNA, 964 bp, Homo sapiens
gcccagtggaattttcctagttctttacactagccatgtatttacctataaaatcaggagaaatatgtatatatataatatattaaaacatatatatatttaaatggggaaatatgtaacaaacaaatagaaacaaggggagaaaggcattgtatttgacaaaacacatatgttcaggtctgagaaggctcataaagaatgttgtctgctatactttgtagttgcttctgttatcacacaatcagtctgcatatacaggcgttttatatatatatttatatagactacatatatacgtatattatatatgtaaatatttcactgtctttgaggacgggggccctgtcttttttatctgtggttttgcttagatgtcctccaacataatcttaacacatagtatgcttttagaaatcgttgactgaatgctaaggacgaaaaaccggtgaccagaaggcaaccaggaaaggctttgctgacctccggagtggtggagttggaggttctgggaaggcgactagggagccaggcaggggcggggtgggatgggatgtggacagcgcttttgcggggggaaagcgtttttgctgctggaattgagcagtaggaatgtgtcagtcacatccccaccttcccaattcttgtcatctcggttcaggaaggtgaacggtgttccgattccccgcggcgggggcctgtagtgggagctctgccccttccccgcctctgctgcaggccccgcccctcgcccggaaccccggggcgctggccgcggtgctgaaacggcgccctccgcggacggaggagggggcggggctctcgggagccgtgagccgggaagagggagacgggcagggcggcgccagcaggccctggtgggcttgggaggaggcaggagactggagacagcctcggctagagcggacacaggcacctggcaagctttccttgaccaaatcaaggt

SEQ ID NO: 10 - DNA, 477 bp, Unknown
  Description of Unknown Synapsin promoter sequence
tctagactgcagagggccctgcgtatgagtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcagtc

SEQ ID NO: 11 - protein, 760 aa, Homo sapiens
MSPKKVQIKVEEKEDETEESSSEEEEEVEDKLPRRESLRPKRKRTRDVINEDDPEPEPEDEETRKAREKERRRRLKRGAEEEEIDEEELERLKAELDEKRQIIATVKCKPWKMEKKIEVLKEAKKFVSENEGALGKGKGKRWFAFKMMMAKKWAKFLRDFENFKAACVPWENKIKAIESQFGSSVASYFLFLRWMYGVNMVLFILTFSLIMLPEYLWGLPYGSLPRKTVPRAEEASAANFGVLYDFNGLAQYSVLFYGYYDNKRTIGWMNFRLPLSYFLVGIMCIGYSFLVVLKAMTKNIGDDGGGDDNTFNFSWKVFTSWDYLIGNPETADNKFNSITMNFKEAITEEKAAQVEENVHLIRFLRFLANFFVFLTLGGSGYLIFWAVKRSQEFAQQDPDTLGWWEKNEMNMVMSLLGMFCPTLFDLFAELEDYHPLIALKWLLGRIFALLLGNLYVFILALMDEINNKIEEEKLVKANITLWEANMIKAYNASFSENSTGPPFFVHPADVPRGPCWETMVGQEFVRLTVSDVLTTYVTILIGDFLRACFVRFCNYCWCWDLEYGYPSYTEFDISGNVLALIFNQGMIWMGSFFAPSLPGINILRLHTSMYFQCWAVMCCNVPEARVFKASRSNNFYLGMLLLILFLSTMPVLYMIVSLPPSFDCGPFSGKNRMFEVIGETLEHDFPSWMAKILRQLSNPGLVIAVILVMVLAIYYLNATAKGQKAANLDLKKKMKMQALENKMRNKKMAAARAAAAAGRQ

SEQ ID NO: 12 - DNA, 3201 bp, Homo sapiens
cagaaactatgagggcagaacccagcaatctgtgctttctttcacaagccctccaggagttgctgaaatttaggaatcattgccccaaaaagtggccctcataatgatgccagatgggatcttactctgttgcccaggctggagtgcagtggtgcgatctcggctctctgcaacctccgcctcccaggttcaagtgattctcctgcctcggcctcctgagtagctgggatttcaggccatgaaagatcactgttttagtctgcgtggtgcagtggaacagatagacctcggtttgaatctcagctctactgtttactagacatgaaatggggaaatctaaaatgagatgccagaagcctcaaaaatggaaaaccccctgtgcttcacatctgaaaatctctgctgggggcagcaactttgagcctgtggggaaggaactgtccacgtggagtggtctggtgaatgcttaaggagctgcagaagggaagtccctctccaaactagccagccactgagaccttctgacaggacacccccaggatgtcacccaaaaaagtacaaatcaaagtggaggaaaaagaagacgagactgaggaaagctcaagtgaagaggaagaggaggtggaagataagctacctcgaagagagagcttgagaccaaagaggaaacggaccagagatgttatcaatgaggatgacccagaacctgaaccagaggatgaagaaacaaggaaggcaagagaaaaagagaggaggaggaggctaaagagaggagcagaagaagaagaaattgatgaagaggaattggaaagattgaaggcagagttagatgagaaaagacaaataattgctactgtcaaatgcaaaccatggaagatggagaagaaaattgaagttctcaaggaggcaaaaaaatttgtgagtgaaaatgaaggggctcttgggaaaggaaaaggaaaacggtggtttgcatttaagatgatgatggccaagaaatgggcaaaattcctccgtgattttgagaacttcaaagctgcgtgtgtcccatgggaaaataaaatcaaggctattgaaagtcagtttggctcctcagtggcctcatacttcctcttcttgagatggatgtatggagtcaatatggttctctttatcctgacatttagcctcatcatgttgccagagtacctctggggtttgccatatggcagtttacctaggaaaaccgttcccagagccgaagaggcatcggcagcaaactttggtgtgttgtacgacttcaatggtttggcacaatattccgttctcttttatggctattatgacaataaacgaacaattggatggatgaatttcaggttgccgctctcctattttctagtggggattatgtgcattggatacagctttctggttgtcctcaaagcaatgaccaaaaacattggtgatgatggaggtggagatgacaacactttcaatttcagctggaaggtctttaccagctgggactacctgatcggcaatcctgaaacagcagacaacaaatttaattctatcacaatgaactttaaggaagctatcacagaagaaaaagcagcccaagtagaagaaaacgtccacttgatcagattcctgaggtttctggctaacttcttcgtgtttctaacacttggagggagtggatacctcatcttttgggctgtgaagcgatcccaggaatttgcacagcaagatcctgacacccttgggtggtgggaaaaaaatgaaatgaacatggttatgtccctcctagggatgttctgtccaacattgtttgacttatttgctgaattagaagactaccatcctctcatcgctttgaaatggctactgggacgcatttttgctcttcttttaggcaatttatacgtatttattcttgcattaatggatgagattaacaacaagattgaagaggagaagctagtaaaggccaatattaccctttgggaagccaatatgatcaaggcctacaatgcatcattctctgaaaatagcactggaccacccttttttgttcaccctgcagatgtacctcgaggaccttgctgggaaacaatggtgggacaggagtttgtgaggctgacagtctctgatgttctgaccacctacgtcacaatcctcattggggactttctaagggcatgttttgtgaggttttgcaattattgctggtgctgggacttggagtatggatatccttcatacaccgaattcgacatcagtggcaacgtcctcgctctgatcttcaaccaaggcatgatctggatgggctccttctttgctcccagcctcccaggcatcaatatccttcgactccatacatccatgtacttccagtgctgggccgttatgtgctgcaatgttcctgaggccagggtcttcaaagcttccagatcaaataacttctacctgggcatgctactgctcatcctcttcctgtccacaatgcctgtcttgtacatgatcgtgtccctcccaccatcttttgattgtggtccattcagtggcaaaaatagaatgtttgaagtcattggagagaccctggagcacgatttcccaagctggatggcgaagatcttgagacagctttcaaaccctgggctggtcattgctgtcattttggtgatggttttggccatctattatctcaatgctactgccaagggccagaaggcagcgaatctggatctcaaaaagaagatgaaaatgcaagctttggagaacaaaatgcgaaacaagaaaatggcagctgcacgagcagctgcagctgctggtcgccagtaataagtatcctgagagcccagaaaaggtacactttgccttgctgtttaaaagtaatgcaatatgtgaacgcccagagaacaagcactgtggaactgctattttcctgttctacccttgatggattttcaaggtcatgctggccaattaaggcatcatcagtcctacctgagcaacaagaatctaaactttattccaagtcagaaactgtttctgcagagccactctctcccctgctccatttcgtgactttttttttttttttaacaaattgagtttagaagtgagtgtaatccagcaatacagtttactggtttagttggtgggttaattaaaaaaaatttgctcatatgaactttcattttatatgtttcttttgcc

SEQ ID NO: 13 - DNA, 20 bp, Artificial Sequence
  Description of Artificial Sequence Synthetic primer
gactatcatatgcttaccgt

SEQ ID NO: 14 - protein, 7 aa, Unknown
  Description of Unknown AAV-PHP.B sequence
TLAVPFK

SEQ ID NO: 15 - DNA, 23 bp, Artificial Sequence
  Description of Artificial Sequence Synthetic oligonucleotide
gggtgggacagaacttccccagg

SEQ ID NO: 16 - DNA, 22 bp, Artificial Sequence
  Description of Artificial Sequence Synthetic oligonucleotide
ggtgggacagaacttccccagg

SEQ ID NO: 17 - DNA, 21 bp, Artificial Sequence
  Description of Artificial Sequence Synthetic oligonucleotide
gtgggacagaacttccccagg

SEQ ID NO: 18 - DNA, 19 bp, Artificial Sequence
  Description of Artificial Sequence Synthetic oligonucleotide
gggacagaacttccccagg

SEQ ID NO: 19 - DNA, 24 bp, Artificial Sequence
  Description of Artificial Sequence Synthetic oligonucleotide
gtgggacagaacttccccaggagg

SEQ ID NO: 20 - DNA, 22 bp, Artificial Sequence
  Description of Artificial Sequence Synthetic oligonucleotide
gggacagaacttccccaggagg

SEQ ID NO: 21 - DNA, 21 bp, Artificial Sequence
  Description of Artificial Sequence Synthetic oligonucleotide
ggacagaacttccccaggagg

SEQ ID NO: 22 - DNA, 20 bp, Artificial Sequence
  Description of Artificial Sequence Synthetic oligonucleotide
gacagaacttccccaggagg

SEQ ID NO: 23 - DNA, 24 bp, Artificial Sequence
  Description of Artificial Sequence Synthetic oligonucleotide
gtggtaatgtccctcctggggaag

SEQ ID NO: 24 - DNA, 22 bp, Artificial Sequence
  Description of Artificial Sequence Synthetic oligonucleotide
ggtaatgtccctcctggggaag

SEQ ID NO: 25 - DNA, 21 bp, Artificial Sequence
  Description of Artificial Sequence Synthetic oligonucleotide
gtaatgtccctcctggggaag

SEQ ID NO: 26 - DNA, 29 bp, Artificial Sequence
  Description of Artificial Sequence Synthetic oligonucleotide
gaacatggtaatgtccctcctggggaagt

SEQ ID NO: 27 - DNA, 28 bp, Artificial Sequence
  Description of Artificial Sequence Synthetic oligonucleotide
gacatggtaatgtccctcctggggaagt

SEQ ID NO: 28 - DNA, 27 bp, Artificial Sequence
  Description of Artificial Sequence Synthetic oligonucleotide
gcatggtaatgtccctcctggggaagt

SEQ ID NO: 29 - DNA, 23 bp, Artificial Sequence
  Description of Artificial Sequence Synthetic oligonucleotide
gggtgggacagaacatccccagg

SEQ ID NO: 30 - DNA, 29 bp, Artificial Sequence
  Description of Artificial Sequence Synthetic oligonucleotide
gaacatggttatgtccctcctagggaagt

SEQ ID NO: 31 - DNA, 28 bp, Artificial Sequence
  Description of Artificial Sequence Synthetic oligonucleotide
gacatggttatgtccctcctagggaagt

SEQ ID NO: 32 - DNA, 27 bp, Artificial Sequence
  Description of Artificial Sequence Synthetic oligonucleotide
gcatggttatgtccctcctagggaagt

SEQ ID NO: 33 - DNA, 20 bp, Artificial Sequence
  Description of Artificial Sequence Synthetic primer
taaagggaccgctctgaaaa

SEQ ID NO: 34 - DNA, 20 bp, Artificial Sequence
  Description of Artificial Sequence Synthetic primer
ccatcaaggcgagaatgaat

SEQ ID NO: 35 - DNA, 20 bp, Artificial Sequence
  Description of Artificial Sequence Synthetic primer
acctcatcttttgggctgtg

SEQ ID NO: 36 - DNA, 12 bp, Artificial Sequence
  Description of Artificial Sequence Synthetic oligonucleotide
tgggacagaaca

SEQ ID NO: 37 - DNA, 12 bp, Artificial Sequence
  Description of Artificial Sequence Synthetic oligonucleotide
tgttctgtccca

SEQ ID NO: 38 - DNA, 12 bp, Artificial Sequence
  Description of Artificial Sequence Synthetic oligonucleotide
tgggacagaact

SEQ ID NO: 39 - DNA, 12 bp, Artificial Sequence
  Description of Artificial Sequence Synthetic oligonucleotide
agttctgtccca

SEQ ID NO: 40 - DNA, 14 bp, Artificial Sequence
  Description of Artificial Sequence Synthetic oligonucleotide
catccccaggaggg

SEQ ID NO: 41 - DNA, 14 bp, Artificial Sequence
  Description of Artificial Sequence Synthetic oligonucleotide
ccctcctggggatg

SEQ ID NO: 42 - DNA, 14 bp, Artificial Sequence
  Description of Artificial Sequence Synthetic oligonucleotide
cttccccaggaggg

SEQ ID NO: 43 - DNA, 14 bp, Artificial Sequence
  Description of Artificial Sequence Synthetic oligonucleotide
ccctcctggggaag

SEQ ID NO: 44 - DNA, 34 bp, Artificial Sequence
  Description of Artificial Sequence Synthetic oligonucleotide
gtttaattgagttgtcatatgttaataacggtat

SEQ ID NO: 45 - DNA, 34 bp, Artificial Sequence
  Description of Artificial Sequence Synthetic oligonucleotide
ataccgttattaacatatgacaactcaattaaac

SEQ ID NO: 46 - DNA, 18 bp, Artificial Sequence
  Description of Artificial Sequence Synthetic oligonucleotide
tccctcctagggaagttc

SEQ ID NO: 47 - DNA, 20 bp, Artificial Sequence
  Description of Artificial Sequence Synthetic oligonucleotide
catcgctttgaaatggctac

SEQ ID NO: 48 - DNA, 20 bp, Artificial Sequence
  Description of Artificial Sequence Synthetic oligonucleotide
aaccatgttcatctacaagg

SEQ ID NO: 49 - DNA, 21 bp, Artificial Sequence
  Description of Artificial Sequence Synthetic primer
agcctagctcagaatcttcca

SEQ ID NO: 50 - DNA, 20 bp, Artificial Sequence
  Description of Artificial Sequence Synthetic primer
aaaatgcgtcccagtagcca

SEQ ID NO: 51 - DNA, 20 bp, Artificial Sequence
  Description of Artificial Sequence Synthetic primer
aaaatgcgtccaagtagcca

SEQ ID NO: 52 - DNA, 21 bp, Artificial Sequence
  Description of Artificial Sequence Synthetic primer
ggaacccctagtgatggagtt

SEQ ID NO: 53 - DNA, 16 bp, Artificial Sequence
  Description of Artificial Sequence Synthetic primer
cggcctcagtgagcga

SEQ ID NO: 54 - DNA, 21 bp, Artificial Sequence
  Description of Artificial Sequence Synthetic probe
cactccctctctgcgcgctcg

SEQ ID NO: 55 - DNA, 25 bp, Artificial Sequence
  Description of Artificial Sequence Synthetic primer
tgaaacatacgttcccaaagagttt

SEQ ID NO: 56 - DNA, 26 bp, Artificial Sequence
  Description of Artificial Sequence Synthetic primer
ctctccttctcagaaagtgtgcatat

SEQ ID NO: 57 - DNA, 27 bp, Artificial Sequence
  Description of Artificial Sequence Synthetic probe
tgctgaaacattcaccttccatgcaga

SEQ ID NO: 58 - DNA, 47 bp, Mus sp.
atgaacatggtaatgtccctcctggggaagttctgtcccaccctgtt

SEQ ID NO: 59 - DNA, 40 bp, Mus sp.
gaacatggtaatgtccctcctggggaagttctgtcccacc

SEQ ID NO: 60 - DNA, 39 bp, Artificial Sequence
  Description of Artificial Sequence Synthetic oligonucleotide
gaacatggtaatgtccctccggggaagttctgtcccacc

SEQ ID NO: 61 - DNA, 39 bp, Artificial Sequence
  Description of Artificial Sequence Synthetic oligonucleotide
gaacatggtaatgtccctctggggaagttctgtcccacc

SEQ ID NO: 62 - DNA, 40 bp, Artificial Sequence
  Description of Artificial Sequence Synthetic oligonucleotide
gaacatggaaatgtccctcctggggaagttctgtcccacc

SEQ ID NO: 63 - DNA, 32 bp, Artificial Sequence
  Description of Artificial Sequence Synthetic oligonucleotide
gaacatggtaattggggaagttctgtcccacc

SEQ ID NO: 64 - DNA, 37 bp, Artificial Sequence
  Description of Artificial Sequence Synthetic oligonucleotide
gaacatggtaatgtccctggggaagttctgtcccacc

SEQ ID NO: 65 - DNA, 40 bp, Artificial Sequence
  Description of Artificial Sequence Synthetic oligonucleotide
gaacatggcaatgtccctcctggggaagttctgtcccacc

SEQ ID NO: 66 - DNA, 40 bp, Mus sp.
gaacatggtaatgtccctcctggggatgttctgtcccacc

SEQ ID NO: 67 - DNA, 40 bp, Artificial Sequence
  Description of Artificial Sequence Synthetic oligonucleotide
gaacatggaaatgtccctcctggggatgttctgtcccacc

SEQ ID NO: 68 - DNA, 27 bp, Artificial Sequence
  Description of Artificial Sequence Synthetic oligonucleotide
  modified_base (22)..(24): a, c, t, g, unknown or other
acatggtaatgtccctcctggnnnrrt

SEQ ID NO: 69 - DNA, 40 bp, Artificial Sequence
  Description of Artificial Sequence Synthetic oligonucleotide
gaacatggtaatgtccctccttggggaagttctgtcccac

SEQ ID NO: 70 - DNA, 40 bp, Artificial Sequence
  Description of Artificial Sequence Synthetic oligonucleotide
gaacatggtaatgtccctccatggggaagttctgtcccac

SEQ ID NO: 71 - DNA, 38 bp, Artificial Sequence
  Description of Artificial Sequence Synthetic oligonucleotide
gaacatggtaatgtcccttggggaagttctgtcccacc

SEQ ID NO: 72 - DNA, 36 bp, Artificial Sequence
  Description of Artificial Sequence Synthetic oligonucleotide
gaacatggtaatgtccctccgaagttctgtcccacc

SEQ ID NO: 73 - DNA, 40 bp, Artificial Sequence
  Description of Artificial Sequence Synthetic oligonucleotide
gaacatgataatgtccctcctggggaagttctgtcccacc

SEQ ID NO: 74 - DNA, 40 bp, Artificial Sequence
  Description of Artificial Sequence Synthetic oligonucleotide
gaacatggtaatgtcactcctggggaagttctgtcccacc

SEQ ID NO: 75 - DNA, 40 bp, Artificial Sequence
  Description of Artificial Sequence Synthetic oligonucleotide
gaacatggtaatgttcctcctggggaagttctgtcccacc

SEQ ID NO: 76 - DNA, 40 bp, Artificial Sequence
  Description of Artificial Sequence Synthetic oligonucleotide
gaacatggtaatgtccctcctgaggaagttctgtcccacc

SEQ ID NO: 77 - DNA, 40 bp, Artificial Sequence
  Description of Artificial Sequence Synthetic oligonucleotide
gaacatggtaatgtccctactggggaagttctgtcccacc

SEQ ID NO: 78 - DNA, 40 bp, Artificial Sequence
  Description of Artificial Sequence Synthetic oligonucleotide
gaacatgataatgtccctcctggggatgttctgtcccacc

SEQ ID NO: 79 - DNA, 40 bp, Artificial Sequence
  Description of Artificial Sequence Synthetic oligonucleotide
gaacatggtaatgttcctcctggggatgttctgtcccacc

SEQ ID NO: 80 - DNA, 40 bp, Artificial Sequence
  Description of Artificial Sequence Synthetic oligonucleotide
gaacatggtaatgtcccccctggggatgttctgtcccacc

SEQ ID NO: 81 - DNA, 40 bp, Artificial Sequence
  Description of Artificial Sequence Synthetic oligonucleotide
gaacatggtaatgtacctcctggggatgttctgtcccacc

SEQ ID NO: 82 - DNA, 40 bp, Artificial Sequence
  Description of Artificial Sequence Synthetic oligonucleotide
gaacatggtactgtccctcctggggatgttctgtcccacc

SEQ ID NO: 83 - DNA, 40 bp, Artificial Sequence
  Description of Artificial Sequence Synthetic oligonucleotide
gaacatggtaatgtccctcccggggatgttctgtcccacc

SEQ ID NO: 84 - DNA, 40 bp, Artificial Sequence
  Description of Artificial Sequence Synthetic oligonucleotide
gaacatggttatgtccctcctggggatgttctgtcctacg

SEQ ID NO: 85 - DNA, 47 bp, Homo sapiens
atgaacatggttatgtccctcctagggaagttctgtccaacattgtt

SEQ ID NO: 86 - DNA, 40 bp, Homo sapiens
gaacatggttatgtccctcctagggaagttctgtccaaca

SEQ ID NO: 87 - DNA, 39 bp, Artificial Sequence
  Description of Artificial Sequence Synthetic oligonucleotide
gaacatggttatgtccctccagggaagttctgtccaaca

SEQ ID NO: 88 - DNA, 20 bp, Artificial Sequence
  Description of Artificial Sequence Synthetic oligonucleotide
gaacatggttatgtccaaca

SEQ ID NO: 89 - DNA, 37 bp, Artificial Sequence
  Description of Artificial Sequence Synthetic oligonucleotide
gaacatggttatgtccctagggaagttctgtccaaca

SEQ ID NO: 90 - DNA, 20 bp, Artificial Sequence
  Description of Artificial Sequence Synthetic oligonucleotide
gaacatggttctgtccaaca

SEQ ID NO: 91 - DNA, 29 bp, Artificial Sequence
  Description of Artificial Sequence Synthetic oligonucleotide
gaacatggttagggaagttctgtccaaca

SEQ ID NO: 92 - DNA, 40 bp, Homo sapiens
gaacatggttatgtccctcctagggatgttctgtccaaca

SEQ ID NO: 93 - DNA, 44 bp, Mus sp.
aacatggtaatgtccctcctggggaagttctgtcccaccctgtt

SEQ ID NO: 94 - DNA, 36 bp, Artificial Sequence
  Description of Artificial Sequence Synthetic oligonucleotide
tgggacagaacttccccaggagggacattaccatgt

SEQ ID NO: 95 - DNA, 36 bp, Artificial Sequence
  Description of Artificial Sequence Synthetic oligonucleotide
  modified_base (12)..(12): a, c, t, g, unknown or other
  modified_base (17)..(18): a, c, t, g, unknown or other
  modified_base (20)..(21): a, c, t, g, unknown or other
  modified_base (23)..(23): a, c, t, g, unknown or other
  modified_base (26)..(26): a, c, t, g, unknown or other
  modified_base (29)..(30): a, c, t, g, unknown or other
  modified_base (32)..(36): a, c, t, g, unknown or other
tgggacagaacntcccnngnngnaanttnncnnnnn

SEQ ID NO: 96 - DNA, 36 bp, Artificial Sequence
  Description of Artificial Sequence Synthetic oligonucleotide
tgggacagaacatccccaggagggacattaccatgt

SEQ ID NO: 97 - DNA, 40 bp, Mus sp.
aacatggtaatgtccctcctggggaagttctgtcccaccc

SEQ ID NO: 98 - DNA, 39 bp, Artificial Sequence
  Description of Artificial Sequence Synthetic oligonucleotide
aacatggtaatgtccctccggggaagttctgtcccaccc

SEQ ID NO: 99 - DNA, 39 bp, Artificial Sequence
  Description of Artificial Sequence Synthetic oligonucleotide
aacatggtaatgtccctcctgggaagttctgtcccaccc

SEQ ID NO: 100 - DNA, 38 bp, Artificial Sequence
  Description of Artificial Sequence Synthetic oligonucleotide
aacatggtaatgtccctcctggaagttctgtcccaccc

SEQ ID NO: 101 - DNA, 40 bp, Artificial Sequence
  Description of Artificial Sequence Synthetic oligonucleotide
aacatggtaatgtccctcctgggggaagttctgtcccacc

SEQ ID NO: 102 - DNA, 38 bp, Artificial Sequence
  Description of Artificial Sequence Synthetic oligonucleotide
aacatggtaatgtccctcggggaagttctgtcccaccc

SEQ ID NO: 103 - DNA, 35 bp, Artificial Sequence
  Description of Artificial Sequence Synthetic oligonucleotide
aacatggtaatgtccctcctagttctgtcccaccc

SEQ ID NO: 104 - DNA, 36 bp, Artificial Sequence
  Description of Artificial Sequence Synthetic oligonucleotide
aacatggtaatgtccctcctgagttctgtcccaccc

SEQ ID NO: 105 - DNA, 40 bp, Artificial Sequence
  Description of Artificial Sequence Synthetic oligonucleotide
acatggtaatgtccctccttggggaagttctgtcccaccc

SEQ ID NO: 106 - DNA, 36 bp, Artificial Sequence
  Description of Artificial Sequence Synthetic oligonucleotide
aacatggtaatgtccctcctaagttctgtcccaccc

SEQ ID NO: 107 - DNA, 40 bp, Artificial Sequence
  Description of Artificial Sequence Synthetic oligonucleotide
aacatggtaatgtccctccttggggaagttctgtcccacc

SEQ ID NO: 108 - DNA, 36 bp, Artificial Sequence
  Description of Artificial Sequence Synthetic oligonucleotide
aacatggtaatgtcccggggaagttctgtcccaccc

SEQ ID NO: 109 - DNA, 37 bp, Artificial Sequence
  Description of Artificial Sequence Synthetic oligonucleotide
aacatggtaatgtccctcctgaagttctgtcccaccc

SEQ ID NO: 110 - DNA, 37 bp, Artificial Sequence
  Description of Artificial Sequence Synthetic oligonucleotide
aacatggtaatgtccctggggaagttctgtcccaccc

SEQ ID NO: 111 - DNA, 39 bp, Artificial Sequence
  Description of Artificial Sequence Synthetic oligonucleotide
aacatggtaatgtccctttggggaagttctgtcccaccc

SEQ ID NO: 112 - DNA, 40 bp, Artificial Sequence
  Description of Artificial Sequence Synthetic oligonucleotide
aacatggtaatgtccctcctggggatgttctgtcccaccc

SEQ ID NO: 113 - DNA, 39 bp, Artificial Sequence
  Description of Artificial Sequence Synthetic oligonucleotide
aacatggtaatgtccctcctgggatgttctgtcccaccc

SEQ ID NO: 114 - DNA, 39 bp, Artificial Sequence
  Description of Artificial Sequence Synthetic oligonucleotide
aacatggtaatgtccctccggggatgttctgtcccaccc

SEQ ID NO: 115 - DNA, 40 bp, Artificial Sequence
  Description of Artificial Sequence Synthetic oligonucleotide
aacatggtaatgtccctcctgggggatgttctgtcccacc

SEQ ID NO: 116 - DNA, 38 bp, Artificial Sequence
  Description of Artificial Sequence Synthetic oligonucleotide
aacatggtaatgtccctcctggatgttctgtcccaccc

SEQ ID NO: 117 - DNA, 34 bp, Artificial Sequence
  Description of Artificial Sequence Synthetic oligonucleotide
aacatggtaatgtccctcctgttctgtcccaccc

SEQ ID NO: 118 - DNA, 38 bp, Artificial Sequence
  Description of Artificial Sequence Synthetic oligonucleotide
aacatggtaatgtccctcggggatgttctgtcccaccc

SEQ ID NO: 119 - DNA, 25 bp, Artificial Sequence
  Description of Artificial Sequence Synthetic oligonucleotide
aacatggtaatgttctgtcccaccc

SEQ ID NO: 120 - DNA, 37 bp, Artificial Sequence
  Description of Artificial Sequence Synthetic oligonucleotide
aacatggtaatgtccctcctgatgttctgtcccaccc

SEQ ID NO: 121 - DNA, 36 bp, Artificial Sequence
  Description of Artificial Sequence Synthetic oligonucleotide
aacatggtaatgtccctcctttgttctgtcccaccc

SEQ ID NO: 122 - DNA, 19 bp, Artificial Sequence
  Description of Artificial Sequence Synthetic oligonucleotide
aacatgttctgtcccaccc

SEQ ID NO: 123 - DNA, 20 bp, Artificial Sequence
  Description of Artificial Sequence Synthetic oligonucleotide
  modified_base (18)..(18): a, c, t, g, unknown or other
gacagaacttccccaggngg

SEQ ID NO: 124 - DNA, 20 bp, Artificial Sequence
  Description of Artificial Sequence Synthetic oligonucleotide
ttatgaactggcacaggtgg

SEQ ID NO: 125 - DNA, 40 bp, Mus sp.
atggtaatgtccctcctggggaagttctgtcccaccctgt

SEQ ID NO: 126 - DNA, 40 bp, Artificial Sequence
  Description of Artificial Sequence Synthetic oligonucleotide
atggtaatgtccctcctggggatgttctgtcccaccctgt

SEQ ID NO: 127 - DNA, 39 bp, Artificial Sequence
  Description of Artificial Sequence Synthetic oligonucleotide
atggtaatgtccctcctgggaagttctgtcccaccctgt

SEQ ID NO: 128 - DNA, 38 bp, Artificial Sequence
  Description of Artificial Sequence Synthetic oligonucleotide
atggtaatgtccctcctgggagttctgtcccaccctgt

SEQ ID NO: 129 - DNA, 39 bp, Artificial Sequence
  Description of Artificial Sequence Synthetic oligonucleotide
atggtaatgtccctcctgggatgttctgtcccaccctgt

SEQ ID NO: 130 - DNA, 36 bp, Artificial Sequence
  Description of Artificial Sequence Synthetic oligonucleotide
atggtaatgtccctcctgggttctgtcccaccctgt

SEQ ID NO: 131 - DNA, 34 bp, Artificial Sequence
  Description of Artificial Sequence Synthetic oligonucleotide
atggtaatgtccctcctgggctgtcccaccctgt

SEQ ID NO: 132 - DNA, 35 bp, Artificial Sequence
  Description of Artificial Sequence Synthetic oligonucleotide
atggtaatgtccctcctgggtctgtcccaccctgt

SEQ ID NO: 133 - DNA, 37 bp, Artificial Sequence
  Description of Artificial Sequence Synthetic oligonucleotide
atggtaatgtccctcctggggttctgtcccaccctgt

SEQ ID NO: 134 - DNA, 38 bp, Artificial Sequence
  Description of Artificial Sequence Synthetic oligonucleotide
atggtaatgtccctcctgggtgttctgtcccaccctgt

SEQ ID NO: 135 - DNA, 27 bp, Mus sp.
acatggtaatgtccctcctggggaagt